(19) United States
(12) Patent Application Publication
(10) Pub. No.: US 2023/0212293 A1
(43) Pub. Date: Jul. 6, 2023

(54) PD-1 AGONIST MULTIMERIC BINDING MOLECULES

(71) Applicant: IGM Biosciences, Inc., Mountain View, CA (US)

(72) Inventors: Bruce KEYT, Hillsborough, CA (US); Todd METZGER, Cupertino, CA (US); Angus SINCLAIR, San Francisco, CA (US)

(21) Appl. No.: 17/996,760

(22) PCT Filed: Apr. 21, 2021
(86) PCT No.: PCT/US2021/028459

§ 371 Date: Oct. 20, 2022

Related U.S. Application Data
(60) Provisional application No. 63/014,023, filed on Apr. 22, 2020.
(60) Provisional application No. 63/050,413, filed on Jul. 10, 2020.
(60) Provisional application No. 63/144,708, filed on Feb. 2, 2021.

(51) Int. Cl. C07K 16/28

(52) U.S. Cl. CPC C07K 16/2818; C07K 2317/35; C07K 2317/75; C07K 2317/565; C07K 2317/52; C07K 2317/94; C07K 2317/92

(57) ABSTRACT

This disclosure provides multimeric binding molecules that specifically and agonistically bind to programmed cell death protein 1 (PD-1). This disclosure also provides compositions comprising the multimeric binding molecules, polynucleotides that encode the multimeric binding molecules, and host cells that can produce the binding molecules. Further this disclosure provides methods of using the multimeric binding molecules, including methods for treating autoimmune disorders and preventing transplantation rejection.

### CROSS-REFERENCE TO RELATED APPLICATIONS

This application claims the benefit of U.S. Provisional Patent Application Serial No. 63/014,023, filed Apr. 22, 2020; 63/050,413, filed Jul. 10, 2020; and 63/144,708, filed Feb. 2, 2021, which are each incorporated herein by reference in their entireties.

### SEQUENCE LISTING

The instant application contains a Sequence Listing which has been submitted electronically in ASCII format and is hereby incorporated by reference in its entirety. The ASCII copy was created on Apr. 20, 2021, is named 031WO1-Sequence-Listing, and is 69,796 bytes in size.

### BACKGROUND

Antibodies and antibody-like molecules that can multimerize, such as IgA and IgM antibodies, have emerged as promising drug candidates, e.g., in the fields of immuno-oncology and infectious diseases, allowing for improved specificity, improved avidity, and the ability to bind to multiple binding targets. See, e.g., U.S. Pat. Nos. 9,951,134, 9,938,347, 10,351,631, and 10,400,038, U.S. Patent Application Publication Nos. US 2019-0100597, US 2018-0009897, US 2019-0330374, US 2019-0330360, US 2019-0338040, US 2019-0338041, US 2019-0185570, US 2018-0265596, US 2018-0118816. US 2018-0118814, and US 2019-0002566, and PCT Publication Nos. WO 2018/187702, WO 2019/165340, and WO 2019/169314, the contents of which are incorporated herein by reference in their entireties.

Programmed cell death protein 1 (PD-1) is a cell surface receptor belonging to the immunoglobulin superfamily, which includes cell surface and soluble proteins that are involved with recognition, binding, and adhesion processes of cells. The initial members of this family were discovered due to their functional effect on augmenting T-cell proliferation following the addition of monoclonal antibodies (Hutloff et al. (1999) Nature 397:263-266; Hansen et al. (1980) Immunogenics 10:247-260). Two cell surface glycoprotein ligands for PD-1, referred to as PD-L1 and PD-L2, have been identified, and have been shown to downregulate T-cell activation and cytokine secretion upon binding to PD-1 (Freeman et al. (2000) J Exp Med 192:1027-34; Latchman et al. (2001) Nat Immunol 2:261-8; Carter et al. (2002) Eur J Immunol 32:634-43; Ohigashi et al. (2005) Clin Cancer Res 11:2947-53). The PD-1 pathway has been implicated in a number of autoimmune diseases (Francisco et al., (2010) Immunol Rev 236: 219-42).

There remains a need for therapeutics to treat autoimmune diseases, such as autoimmune diseases resulting from disruption of the PD-1 pathway.

### SUMMARY

Provided herein is a multimeric binding molecule comprising two, five, or six bivalent binding units or variants or fragments thereof, where each binding unit comprises two IgA or IgM heavy chain constant regions or multimerizing fragments or variants thereof, each associated with a binding domain, where three to twelve of the binding domains are programmed cell death protein 1 (PD-1)-binding domains that specifically and agonistically bind to PD-1, where the binding molecule can activate PD-1-mediated signal transduction in a cell at a higher potency than an equivalent amount of a bivalent IgG antibody or fragment thereof comprising two of the same PD-1-binding domains, which also specifically binds to and agonizes PD-1. In some embodiments, the two, five, or six binding units are human, humanized, or chimeric immunoglobulin binding units.

In some embodiments, the three to twelve PD-1-binding domains comprise a heavy chain variable region (VH) and a light chain variable region (VL), where the VH and VL comprise six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 comprise the CDRs of an antibody comprising the VH and VL of SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, or SEQ ID NO: 49 and SEQ ID NO: 50, respectively, or the VH of any one of SEQ ID NO: 15, SEQ ID NO: 17, SEQ ID NO: 19, SEQ ID NO: 21, SEQ ID NO: 23, or SEQ ID NO: 24 and the VL of any one of SEQ ID NO: 16. SEQ ID NO: 18, SEQ ID NO: 20, or SEQ ID NO: 22, or the CDRs of an antibody comprising the VH and VL of SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, or SEQ ID NO: 49 and SEQ ID NO: 50, respectively with one or two single amino acid substitutions in one or more of the HCDRs or LCDRs, or the VH of any one of SEQ ID NO: 15, SEQ ID NO: 17, SEQ ID NO: 19, SEQ ID NO: 21, SEQ ID NO: 23, or SEQ ID NO: 24 and the VL of any one of SEQ ID NO: 16, SEQ ID NO: 18, SEQ ID NO: 20, or SEQ ID NO: 22 with one or two single amino acid substitutions in one or more of the HCDRs or LCDRs.

In some embodiments, the three to twelve PD-1-binding domains comprise a heavy chain variable region (VH) and a light chain variable region (VL), where: (a) the VH and VL comprise six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 comprise the CDRs of an antibody comprising the VH and VL of SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14. SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, or SEQ ID NO: 49 and SEQ ID NO: 50, respectively with zero, one, or two single amino acid substitutions in one or more of the HCDRs or LCDRs; (b) the VH and VL comprise six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 comprise the CDRs of an antibody comprising the VH of any one of SEQ ID NO: 15, SEQ ID NO: 17, SEQ ID NO: 19, SEQ ID NO: 21, SEQ ID NO: 23, or SEQ ID NO: 24 and the VL of any one of SEQ ID NO: 16, SEQ ID NO: 18, SEQ ID NO: 20, or SEQ ID NO: 22 with zero, one, or two single amino acid substitutions in one or more of the HCDRs or LCDRs; (c) the VH and VL comprise amino acid sequences at least 80%, at least 85%, at least 90%, at least 95% or 100% identical to SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, or SEQ ID NO: 49 and SEQ ID NO: 50, respectively; or (d) the VH comprises an amino acid sequence at least 80%, at least 85%, at least 90%, at least 95% or 100% identical to of any one of SEQ ID NO: 15. SEQ ID NO: 17, SEQ ID NO: 19, SEQ ID NO: 21, SEQ ID NO: 23, or SEQ ID NO: 24 and the VL comprises an amino acid sequence at least 80%, at least 85%, at least 90%, at least 95% or 100% identical to of any one of SEQ ID NO: 16. SEQ ID NO: 18, SEQ ID NO: 20, or SEQ ID NO: 22.

In some embodiments, the VH and VL comprise six immunoglobulin complementarity determining regions HCDR1, HCDR2, HCDR3, LCDR1, LCDR2, and LCDR3, where the HCDR1, HCDR2, HCDR3, LCDR1, LCDR2, and LCDR3 comprise the CDRs of an antibody comprising the VH and VL of SEQ ID NO: 1 and SEQ ID NO: 2, SEQ ID NO: 3 and SEQ ID NO: 4, SEQ ID NO: 13 and SEQ ID NO: 14, and SEQ ID NO: 25 and SEQ ID NO: 26, respectively, or the CDRs of an antibody comprising the VH and VL of SEQ ID NO: 1 and SEQ ID NO: 2, SEQ ID NO: 3 and SEQ ID NO: 4, SEQ ID NO: 13 and SEQ ID NO: 14, and SEQ ID NO: 25 and SEQ ID NO: 26, respectively with one or two single amino acid substitutions in one or more of the HCDRs or LCDRs.

In some embodiments, the three to twelve PD-1-binding domains of the binding molecule comprise an antibody VH and a VL, where the VH and VL comprise amino acid sequences at least 80%, at least 85%, at least 90%, at least 95% or 100% identical to SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 25 and SEQ ID NO: 26, SEQ ID NO: 27 and SEQ ID NO: 28, SEQ ID NO: 29 and SEQ ID NO: 30. SEQ ID NO: 31 and SEQ ID NO: 32, SEQ ID NO: 33 and SEQ ID NO: 34, or SEQ ID NO: 49 and SEQ ID NO: 50, respectively, or the VH of any one of SEQ ID NO: 15, SEQ ID NO: 17, SEQ ID NO: 19, SEQ ID NO: 21, SEQ ID NO: 23, or SEQ ID NO: 24 and the VL of any one of SEQ ID NO: 16, SEQ ID NO: 18, SEQ ID NO: 20, or SEQ ID NO: 22. In some embodiments, the three to twelve PD-1-binding domains of the binding molecule comprise an antibody VH and a VL, where the VH and VL comprise amino acid sequences at least 80%, at least 85%, at least 90%, at least 95% or 100% identical to SEQ ID NO: 1 and SEQ ID NO: 2, SEQ ID NO: 3 and SEQ ID NO: 4, SEQ ID NO: 13 and SEQ ID NO: 14, and SEQ ID NO: 25 and SEQ ID NO: 26, respectively.

In some embodiments, the three to twelve PD-1-binding domains comprise antibody VH and VL regions comprising the amino acid sequences SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, or SEQ ID NO: 49 and SEQ ID NO: 50, respectively, or the VH of any one of SEQ ID NO: 15, SEQ ID NO: 17, SEQ ID NO: 19, SEQ ID NO: 21, SEQ ID NO: 23, or SEQ ID NO: 24 and the VL of any one of SEQ ID NO: 16, SEQ ID NO: 18, SEQ ID NO: 20, or SEQ ID NO: 22. In some embodiments, the three to twelve PD-1-binding domains comprise antibody VH and VL regions comprising the amino acid sequences SEQ ID NO: 1 and SEQ ID NO: 2, SEQ ID NO: 3 and SEQ ID NO: 4, SEQ ID NO: 13 and SEQ ID NO: 14, or SEQ ID NO: 25 and SEQ ID NO: 26, respectively.

In some embodiments, each binding unit comprises two heavy chains and two light chains, where the heavy chains and light chains comprise VH and VL amino acid sequences at least 80%, at least 85%, at least 90%, at least 95% or 100% identical to SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, or SEQ ID NO: 49 and SEQ ID NO: 50, respectively, or the VH of any one of SEQ ID NO: 15, SEQ ID NO: 17, SEQ ID NO: 19, SEQ ID NO: 21, SEQ ID NO: 23, or SEQ ID NO: 24 and the VL of any one of SEQ ID NO: 16. SEQ ID NO: 18, SEQ ID NO: 20, or SEQ ID NO: 22. In some embodiments, each binding unit comprises two heavy chains and two light chains, where the heavy chains and light chains comprise VH and VL amino acid sequences at least 80%, at least 85%, at least 90%, at least 95% or 100% identical to SEQ ID NO: 1 and SEQ ID NO: 2, SEQ ID NO: 3 and SEQ ID NO: 4, SEQ ID NO: 13 and SEQ ID NO: 14, or SEQ ID NO: 25 and SEQ ID NO: 26, respectively.

In some embodiments, the heavy chains and light chains comprise the VH and VL amino acid sequences SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, or SEQ ID NO: 49 and SEQ ID NO: 50, respectively, or the VH of any one of SEQ ID NO: 15, SEQ ID NO: 17, SEQ ID NO: 19, SEQ ID NO: 21, SEQ ID NO: 23, or SEQ ID NO: 24 and the VL of any one of SEQ ID NO: 16, SEQ ID NO: 18, SEQ ID NO: 20, or SEQ ID NO: 22. In some embodiments, the heavy chains and light chains comprise the VH and VL amino acid sequences SEQ ID NO: 1 and SEQ ID NO: 2, SEQ ID NO: 3 and SEQ ID NO: 4, SEQ ID NO: 13 and SEQ ID NO: 14, or SEQ ID NO: 25 and SEQ ID NO: 26, respectively.

In some embodiments, the multimeric binding molecule is a dimeric binding molecule comprising two bivalent IgA or IgA-like binding units and a J chain or functional fragment or variant thereof, where each binding unit comprises two IgA heavy chain constant regions or multimerizing fragments or variants thereof, each comprising an IgA Cα3 domain and an IgA tailpiece domain. In some embodiments, each IgA heavy chain constant region or multimerizing fragment or variant thereof further comprises a Cal domain, a Cα2 domain, an IgA hinge region, or any combination thereof. In some embodiments, the IgA heavy chain constant regions or multimerizing fragments thereof are human IgA constant regions. In some embodiments, each binding unit comprises two IgA heavy chains each comprising a VH situated amino terminal to the IgA constant region or multimerizing fragment thereof, and two immunoglobulin light chains each comprising a VL situated amino terminal to an immunoglobulin light chain constant region.

In some embodiments, the multimeric binding molecule is a pentameric or a hexameric binding molecule comprising five or six bivalent IgM binding units, respectively, where each binding unit comprises two IgM heavy chain constant regions or multimerizing fragments thereof each associated with a PD-1-binding domain, where each IgM heavy chain constant region comprises an IgM Cμ4 and IgM tailpiece domain. In some embodiments, the IgM heavy chain constant regions or fragments or variants thereof each further comprise a Cμ1 domain, a Cμ2 domain, a Cμ3 domain, or any combination thereof. In some embodiments, the IgM heavy chain constant region is a human IgM constant region.

In some embodiments, each binding unit comprises two IgM heavy chains each comprising a VH situated amino terminal to the IgM constant region or fragment thereof, and two immunoglobulin light chains each comprising a VL situated amino terminal to an immunoglobulin light chain constant region.

In some embodiments, the multimeric binding molecule comprises SEQ ID NO: 35, SEQ ID NO: 36, or a multimerizing fragment thereof. In some embodiments, the IgM constant region comprises a substitution relative to a wild-type human IgM constant region at position 310, 311, 313, and/or 315 of SEQ ID NO: 35 or SEQ ID NO: 36. In some embodiments, the IgM constant region comprises two or more substitutions relative to a wild-type human IgM constant region at positions 46, 209, 272, or 440 of SEQ ID NO: 35 or SEQ ID NO: 36.

In some embodiments, the multimeric binding molecule is pentameric, and further comprises a J-chain or functional fragment or variant thereof.

In some embodiments, the J-chain or functional fragment or variant thereof is a variant J-chain comprising one or more single amino acid substitutions, deletions, or insertions relative to a wild-type J-chain that can affect serum half-life of the multimeric binding molecule; and where the multimeric binding molecule comprising the variant J-chain exhibits an increased serum half-life upon administration to an animal relative to a reference multimeric binding molecule that is identical except for the one or more single amino acid substitutions, deletions, or insertions, and is administered in the same way to the same animal species.

In some embodiments, the J-chain or functional fragment thereof comprises an amino acid substitution at the amino acid position corresponding to amino acid Y102 of the mature wild-type human J-chain (SEQ ID NO: 41). In some embodiments, the amino acid corresponding to Y102 of SEQ ID NO: 41 is substituted with alanine (A), serine (S), or arginine (R). In some embodiments, the amino acid corresponding to Y102 of SEQ ID NO: 41 is substituted with alanine (A).

In some embodiments, the J-chain is a variant human J-chain and comprises the amino acid sequence SEQ ID NO: 42. In some embodiments, the J-chain or functional fragment thereof comprises an amino acid substitution at the amino acid position corresponding to amino acid N49, amino acid S51, or both N49 and S51 of the mature human J-chain (SEQ ID NO: 41), where a single amino acid substitution corresponding to position S51 of SEQ ID NO: 41 is not a threonine (T) substitution. In some embodiments, the position corresponding to N49 of SEQ ID NO: 41 is substituted with alanine (A), glycine (G), threonine (T), serine (S) or aspartic acid (D). In some embodiments, the position corresponding to N49 of SEQ ID NO: 41 or SEQ ID NO: 42 is substituted with alanine (A). In some embodiments, the position corresponding to S51 of SEQ ID NO: 41 or SEQ ID NO: 42 is substituted with alanine (A) or glycine (G). In some embodiments, the position corresponding to S51 of SEQ ID NO: 41 or SEQ ID NO: 42 is substituted with alanine (A).

In some embodiments, the J-chain or functional fragment or variant thereof further comprises a heterologous polypeptide, where the heterologous polypeptide is directly or indirectly fused to the J-chain or functional fragment or variant thereof.

In some embodiments, the heterologous polypeptide is fused to the J-chain or fragment thereof via a peptide linker. In some embodiments, the peptide linker comprises at least 5 amino acids, but no more than 25 amino acids. In some embodiments, the J-chain or functional fragment or variant thereof further comprises a heterologous polypeptide, where the heterologous polypeptide is fused to the J-chain or functional fragment or variant thereof via a peptide linker comprising at least 5 amino acids, but no more than 25 amino acids. In some embodiments, the peptide linker consists of GGGGS (SEQ ID NO: 43), GGGGSGGGGS (SEQ ID NO: 44), GGGGSGGGGSGGGGS (SEQ ID NO: 45), GGGGSGGGGSGGGGSGGGGS (SEQ ID NO: 46), or GGGGSGGGGSGGGGSGGGGSGGGGS (SEQ ID NO: 47). In some embodiments, the heterologous polypeptide is fused to the N-terminus of the J-chain or fragment or variant thereof, the C-terminus of the J-chain or fragment or variant thereof, or to both the N-terminus and C-terminus of the J-chain or fragment or variant thereof.

In some embodiments, the heterologous polypeptide can influence the absorption, distribution, metabolism and/or excretion (ADME) of the multimeric binding molecule.

In some embodiments, the heterologous polypeptide comprises an antigen binding domain. In some embodiments, the antigen binding domain of the heterologous polypeptide is an antibody or antigen-binding fragment thereof. In some embodiments, the antigen-binding fragment comprises an Fab fragment, an Fab′ fragment, an F(ab′)2 fragment, an Fd fragment, an Fv fragment, a single-chain Fv (scFv) fragment, a disulfide-linked Fv (sdFv) fragment, or any combination thereof. In some embodiments, the antigen-binding fragment is a scFv fragment. In some embodiments, the antigen binding domain binds ICOS Ligand (ICOSLG), ICOS (CD278), Interleukin 6 (IL6), CD28, CD3, CD80, CD86, Tumor Necrosis Factor Alpha (TNFa), or Fibroblast Activation Protein (FAP).

Also provided herein is a composition comprising the multimeric binding molecule disclosed herein. In some embodiments, the composition further comprises a pharmacologically acceptable excipient.

Also provided herein is a polynucleotide comprising a nucleic acid sequence that encodes a polypeptide subunit of the binding molecule disclosed herein. In some embodiments, the polypeptide subunit comprises an IgM heavy chain constant region and at least an antibody VH portion of the PD-1-binding domain of the multimeric binding molecule.

In some embodiments, the polypeptide subunit comprises a human IgM constant region or fragment thereof fused to the C-terminal end of a VH comprising: (a) HCDR1, HCDR2, and HCDR3 regions comprising the CDRs contained in the VH amino acid sequences SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 24, SEQ ID NO: 25, SEQ ID NO: 27, SEQ ID NO: 29, SEQ ID NO: 31, SEQ ID NO: 33, or SEQ ID NO: 49, or the CDRs contained in the VH amino acid sequences SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19. SEQ ID NO: 21, SEQ ID NO: 23. SEQ ID NO: 24, SEQ ID NO: 25. SEQ ID NO: 27, SEQ ID NO: 29, SEQ ID NO: 31, SEQ ID NO: 33, or SEQ ID NO: 49 with one or two single amino acid substitutions in one or more of the HCDRs; or (b) an amino acid sequence at least 80%, at least 85%, at least 90%, at least 95% or 100% identical to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 24, SEQ ID NO: 25, SEQ ID NO: 27, SEQ ID NO: 29, SEQ ID NO: 31, SEQ ID NO: 33, or SEQ ID NO: 49.

In some embodiments, the polypeptide subunit comprises a light chain constant region and an antibody VL portion of the PD-1-binding domain of the multimeric binding molecule. In some embodiments, the polypeptide subunit comprises a human kappa or lambda light chain constant region or fragment thereof fused to the C-terminal end of a VL comprising: (a) LCDR1, LCDR2, and LCDR3 regions comprising the CDRs contained in the VL amino acid sequences SEQ ID NO: 2, SEQ ID NO: 4, SEQ ID NO: 6, SEQ ID NO: 8, SEQ ID NO: 10, SEQ ID NO: 12. SEQ ID NO: 14, SEQ ID NO: 16. SEQ ID NO: 18, SEQ ID NO: 20, SEQ ID NO: 22, SEQ ID NO: 26, SEQ ID NO: 28, SEQ ID NO: 30, SEQ ID NO: 32, SEQ ID NO: 34, or SEQ ID NO: 50, or the CDRs contained in the VL amino acid sequences SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 26, SEQ ID NO: 28, SEQ ID NO: 30, SEQ ID NO: 32, SEQ ID NO: 34, or SEQ ID NO: 50 with one or two single amino acid substitutions in one or more of the LCDRs; or (b) an amino acid sequence at least 80%, at least 85%, at least 90%, at least 95% or 100% identical to SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 26, SEQ ID NO: 28, SEQ ID NO: 30, SEQ ID NO: 32, SEQ ID NO: 34, or SEQ ID NO: 50.

Also provided herein is a composition comprising a polynucleotide provided herein. In some embodiments, the composition comprises a polynucleotide comprising a nucleic acid sequence that encodes an IgM heavy chain constant region and at least an antibody VH portion of the PD-1-binding domain of a multimeric binding molecule provided herein, and a polynucleotide comprising a nucleic acid sequence that encodes a light chain constant region and an antibody VL portion of the PD-1-binding domain of a multimeric binding molecule provided herein. In some embodiments, the polynucleotides are on separate vectors. In some embodiments, the polynucleotides are on a single vector. In some embodiments, the composition further comprises a polynucleotide comprising a nucleic acid sequence encoding a J chain, or a functional fragment thereof, or a functional variant thereof.

Also provided herein is a vector or vectors disclosed herein.

Also provided herein is a host cell comprising a polynucleotide provided herein, a composition provided herein, or a vector or vectors provided herein, where the host cell can express a binding molecule provided herein, or a subunit thereof.

Also provided herein is a method of producing a binding molecule provided herein, comprising culturing a host cell provided herein, and recovering the binding molecule.

Also provided herein is a method for treating an autoimmune disorder, an inflammatory disorder, or a combination thereof in a subject in need of treatment comprising administering to the subject an effective amount of a multimeric binding molecule provided herein, where the multimeric binding molecule exhibits greater potency than an equivalent amount of a monomeric or dimeric binding molecule binding to the same binding partner. In some embodiments, the subject is human.

Also provided herein is a method for preventing transplantation rejection in a subject, comprising administering to the subject an effective amount of a multimeric binding molecule provided herein, where the multimeric binding molecule exhibits greater potency than an equivalent amount of a monomeric or dimeric binding molecule binding to the same binding partner, and where the subject is a transplantation recipient. In some embodiments, the subject is human.

### BRIEF DESCRIPTION OF THE DRAWINGS/FIGURES

FIG. 1 shows binding of anti-PD-1 IgG #1, anti-PD-1 IgM #1, anti-PD-1 IgG #2, and anti-PD-1 IgM #2 to human PD-1 in an ELISA assay.

FIG. 2 shows binding of anti-PD-1 IgG #1, anti-PD-1 IgM #1, anti-PD-1 IgG #2, and anti-PD-1 IgM #2 to human PD-1 in a cell-based assay.

FIGS. 3A-3B show activation of PD-1 signaling by anti-PD-1 IgG or IgM #1 (FIG. 3A) or anti-PD-1 IgG or IgM #2 (FIG. 3B).

FIGS. 4A-4B show activation of PD-1 signaling by anti-PD-1 IgG, IgG+cross-linker, or IgM #1 (FIG. 4A) or anti-PD-1 IgG, IgG+cross-linker, or IgM #2 (FIG. 4B).

FIGS. 5A-5D show activation of PD-1 signaling by anti-PD-1 IgG, IgG+cross-linker, or IgM #1 (FIG. 5A) or anti-PD-1 IgG, IgG+cross-linker, or IgM #2 (FIG. 5B) or anti-PD-1 IgG, IgG+cross-linker, or IgM #3 (FIG. 5C) or anti-PD-1 IgG, IgG+cross-linker, or IgM #4 (FIG. 5D).

FIGS. 6A-6D show activation of PD-1 signaling by anti-PD-1 IgG, IgG+cross-linker, pentameric IgM, or hexameric IgHM #1 (FIG. 6A) or anti-PD-1 IgG, IgG+cross-linker, pentameric IgM, or hexameric IgHM #2 (FIG. 6B) or anti-PD-1 IgG, IgG+cross-linker, pentameric IgM, or hexameric IgHM #3 (FIG. 6C) or anti-PD-1 IgG, IgG+cross-linker, pentameric IgM, or hexameric IgHM #5 (FIG. 6D).

### DETAILED DESCRIPTION

### Definitions

As used herein, the term “a” or “an” entity refers to one or more of that entity; for example, “a binding molecule,” is understood to represent one or more binding molecules. As such, the terms “a” (or “an”), “one or more,” and “at least one” can be used interchangeably herein.

Furthermore, “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Unless defined otherwise, technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry and Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, amino acid sequences are written left to right in amino to carboxy orientation. The headings provided herein are not limitations of the various embodiments or embodiments of the disclosure, which can be had by reference to the specification as a whole. Accordingly, the terms defined immediately below are more fully defined by reference to the specification in its entirety.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids and does not refer to a specific length of the product. Thus, peptides, dipeptides, tripeptides, oligopeptides, “protein,” “amino acid chain.” or any other term used to refer to a chain or chains of two or more amino acids are included within the definition of “polypeptide,” and the term “polypeptide” can be used instead of any of these terms. The term “polypeptide” is also intended to refer to the products of post-expression modifications of the polypeptide, including without limitation glycosylation, acetylation, phosphorylation, amidation, and derivatization by known protecting/blocking groups, proteolytic cleavage, or modification by non-naturally occurring amino acids. A polypeptide can be derived from a biological source or produced by recombinant technology but is not necessarily translated from a designated nucleic acid sequence. It can be generated in any manner, including by chemical synthesis.

A polypeptide as disclosed herein can be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides can have a defined three-dimensional structure, although they do not necessarily have such structure. Polypeptides with a defined three-dimensional structure are referred to as folded, and polypeptides which do not possess a defined three-dimensional structure, but rather can adopt many different conformations and are referred to as unfolded. As used herein, the term glycoprotein refers to a protein coupled to at least one carbohydrate moiety that is attached to the protein via an oxygen-containing or a nitrogen-containing side chain of an amino acid, e.g., a serine or an asparagine. Asparagine (N)-linked glycans are described in more detail elsewhere in this disclosure.

By an “isolated” polypeptide or a fragment, variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated as disclosed herein, as are native or recombinant polypeptides which have been separated, fractionated, or partially or substantially purified by any suitable technique.

As used herein, the term “a non-naturally occurring polypeptide” or any grammatical variants thereof, is a conditional definition that explicitly excludes, but only excludes, those forms of the polypeptide that are, or might be, determined or interpreted by a judge or an administrative or judicial body, to be “naturally-occurring.”

Other polypeptides disclosed herein are fragments, derivatives, analogs, or variants of the foregoing polypeptides, and any combination thereof. The terms “fragment,” “variant,” “derivative” and “analog” as disclosed herein include any polypeptides which retain at least some of the properties of the corresponding native antibody or polypeptide, for example, specifically binding to an antigen. Fragments of polypeptides include, for example, proteolytic fragments, as well as deletion fragments, in addition to specific antibody fragments discussed elsewhere herein. Variants of, e.g., a polypeptide include fragments as described above, and also polypeptides with altered amino acid sequences due to amino acid substitutions, deletions, or insertions. In certain embodiments, variants can be non-naturally occurring. Non-naturally occurring variants can be produced using art-known mutagenesis techniques. Variant polypeptides can comprise conservative or non-conservative amino acid substitutions, deletions, or additions. Derivatives are polypeptides that have been altered so as to exhibit additional features not found on the original polypeptide. Examples include fusion proteins. As used herein a “derivative” of a polypeptide can also refer to a subject polypeptide having one or more amino acids chemically derivatized by reaction of a functional side group. Also included as “derivatives” are those polypeptides that contain one or more derivatives of the twenty standard amino acids. For example, 4-hydroxyproline can be substituted for proline; 5-hydroxylysine can be substituted for lysine; 3-methylhistidine can be substituted for histidine; homoserine can be substituted for serine; and omithine can be substituted for lysine.

A “conservative amino acid substitution” is one in which one amino acid is replaced with another amino acid having a similar side chain. Families of amino acids having similar side chains have been defined in the art, including basic side chains (e.g., lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g., glycine, alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. In certain embodiments, conservative substitutions in the sequences of the polypeptides, binding molecules, and antibodies of the present disclosure do not abrogate the binding of the polypeptide, binding molecule, or antibody containing the amino acid sequence, to the antigen to which the antibody binds. Methods of identifying nucleotide and amino acid conservative substitutions which do not eliminate antigen-binding are well-known in the art (see, e.g., Brummell et al., Biochem. 32: 1180-1187 (1993); Kobayashi et al., Protein Eng. 12(10):879-884 (1999); and Burks et al., Proc. Natl. Acad. Sci. USA 94: 412-417 (1997)).

The term “polynucleotide” is intended to encompass a singular nucleic acid as well as plural nucleic acids and refers to an isolated nucleic acid molecule or construct, e.g., messenger RNA (mRNA), cDNA, or plasmid DNA (pDNA). A polynucleotide can comprise a conventional phosphodiester bond or a non-conventional bond (e.g., an amide bond, such as found in peptide nucleic acids (PNA)). The terms “nucleic acid” or “nucleic acid sequence” refer to any one or more nucleic acid segments, e.g., DNA or RNA fragments, present in a polynucleotide.

By an “isolated” nucleic acid or polynucleotide is intended any form of the nucleic acid or polynucleotide that is separated from its native environment. For example, gel-purified polynucleotide, or a recombinant polynucleotide encoding a polypeptide contained in a vector would be considered to be “isolated.” Also, a polynucleotide segment, e.g., a PCR product, which has been engineered to have restriction sites for cloning is considered to be “isolated.” Further examples of an isolated polynucleotide include recombinant polynucleotides maintained in heterologous host cells or purified (partially or substantially) polynucleotides in a non-native solution such as a buffer or saline. Isolated RNA molecules include in vivo or in vitro RNA transcripts of polynucleotides, where the transcript is not one that would be found in nature. Isolated polynucleotides or nucleic acids further include such molecules produced synthetically. In addition, polynucleotide or a nucleic acid can be or can include a regulatory element such as a promoter, ribosome binding site, or a transcription terminator.

As used herein, the term “a non-naturally occurring polynucleotide” or any grammatical variants thereof, is a conditional definition that explicitly excludes, but only excludes, those forms of the nucleic acid or polynucleotide that are, or might be, determined or interpreted by a judge, or an administrative or judicial body, to be “naturally-occurring.”

As used herein, a “coding region” is a portion of nucleic acid which consists of codons translated into amino acids. Although a “stop codon” (TAG, TGA, or TAA) is not translated into an amino acid, it can be considered to be part of a coding region, but any flanking sequences, for example promoters, ribosome binding sites, transcriptional terminators, introns, and the like, are not part of a coding region. Two or more coding regions can be present in a single polynucleotide construct, e.g., on a single vector, or in separate polynucleotide constructs, e.g., on separate (different) vectors. Furthermore, any vector can contain a single coding region, or can comprise two or more coding regions, e.g., a single vector can separately encode an immunoglobulin heavy chain variable region and an immunoglobulin light chain variable region. In addition, a vector, polynucleotide, or nucleic acid can include heterologous coding regions, either fused or unfused to another coding region. Heterologous coding regions include without limitation, those encoding specialized elements or motifs, such as a secretory signal peptide or a heterologous functional domain.

In certain embodiments, the polynucleotide or nucleic acid is DNA. In the case of DNA, a polynucleotide comprising a nucleic acid which encodes a polypeptide normally can include a promoter and/or other transcription or translation control elements operably associated with one or more coding regions. An operable association is when a coding region for a gene product, e.g., a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter can be a cell-specific promoter that directs substantial transcription of the DNA in predetermined cells. Other transcription control elements, besides a promoter, for example enhancers, operators, repressors, and transcription termination signals, can be operably associated with the polynucleotide to direct cell-specific transcription.

A variety of transcription control regions are known to those skilled in the art. These include, without limitation, transcription control regions that function in vertebrate cells, such as, but not limited to, promoter and enhancer segments from cytomegaloviruses (the immediate early promoter, in conjunction with intron-A), simian virus 40 (the early promoter), and retroviruses (such as Rous sarcoma virus). Other transcription control regions include those derived from vertebrate genes such as actin, heat shock protein, bovine growth hormone and rabbit ß-globin, as well as other sequences capable of controlling gene expression in eukaryotic cells. Additional suitable transcription control regions include tissue-specific promoters and enhancers as well as lymphokine-inducible promoters (e.g., promoters inducible by interferons or interleukins).

Similarly, a variety of translation control elements are known to those of ordinary skill in the art. These include, but are not limited to ribosome binding sites, translation initiation and termination codons, and elements derived from picornaviruses (particularly an internal ribosome entry site, or IRES, also referred to as a CITE sequence).

In other embodiments, a polynucleotide can be RNA, for example, in the form of messenger RNA (mRNA), transfer RNA, or ribosomal RNA.

Polynucleotide and nucleic acid coding regions can be associated with additional coding regions which encode secretory or signal peptides, which direct the secretion of a polypeptide encoded by a polynucleotide as disclosed herein. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells can have a signal peptide fused to the N-terminus of the polypeptide, which is cleaved from the complete or “full length” polypeptide to produce a secreted or “mature” form of the polypeptide. In certain embodiments, the native signal peptide, e.g., an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, can be used. For example, the wild-type leader sequence can be substituted with the leader sequence of human tissue plasminogen activator (TPA) or mouse ß-glucuronidase.

As used herein, the term “binding molecule” refers in its broadest sense to a molecule that specifically binds to a receptor or target, e.g., an epitope or an antigenic determinant. As described further herein, a binding molecule can comprise one of more “binding domains,” e.g., “antigen-binding domains” described herein. A non-limiting example of a binding molecule is an antibody or antibody-like molecule as described in detail herein that retains antigen-specific binding. In certain embodiments a “binding molecule” comprises an antibody or antibody-like or antibody-derived molecule as described in detail herein.

As used herein, the terms “binding domain” or “antigen-binding domain” (can be used interchangeably) refer to a region of a binding molecule, e.g., an antibody or antibody-like, or antibody-derived molecule, that is necessary and sufficient to specifically bind to a target, e.g., an epitope, a polypeptide, a cell, or an organ. For example, an “Fv,” e.g., a heavy chain variable region and a light chain variable region of an antibody, either as two separate polypeptide subunits or as a single chain, is considered to be a “binding domain.” Other antigen-binding domains include, without limitation, a single domain heavy chain variable region (VHH) of an antibody derived from a camelid species, or six immunoglobulin complementarity determining regions (CDRs) expressed in a fibronectin scaffold. A “binding molecule,” e.g., an “antibody” as described herein can include one, two, three, four, five, six, seven, eight, nine, ten, eleven, twelve, or more “antigen-binding domains.”

The terms “antibody” and “immunoglobulin” can be used interchangeably herein. An antibody (or a fragment, variant, or derivative thereof as disclosed herein, e.g., an IgM-like antibody) includes at least the variable domain of a heavy chain (e.g., from a camelid species) or at least the variable domains of a heavy chain and a light chain. Basic immunoglobulin structures in vertebrate systems are relatively well understood. See. e.g., Harlow et al., Antibodies: A Laboratory Manual, (Cold Spring Harbor Laboratory Press, 2nd ed. 1988). Unless otherwise stated, the term “antibody” encompasses anything ranging from a small antigen-binding fragment of an antibody to a full sized antibody, e.g., an IgG antibody that includes two complete heavy chains and two complete light chains, an IgA antibody that includes four complete heavy chains and four complete light chains and includes a J-chain and/or a secretory component, or an IgM-derived binding molecule, e.g., an IgM antibody or IgM-like antibody, that includes ten or twelve complete heavy chains and ten or twelve complete light chains and optionally includes a J-chain or functional fragment or variant thereof.

The term “immunoglobulin” comprises various broad classes of polypeptides that can be distinguished biochemically. Those skilled in the art will appreciate that heavy chains are classified as gamma, mu, alpha, delta, or epsilon, (γ, α, α, δ, ε) with some subclasses among them (e.g., γ1-γ4 or α1-α2)). It is the nature of this chain that determines the “isotype” of the antibody as IgG, IgM, IgA IgD, or IgE, respectively. The immunoglobulin subclasses (subtypes) e.g., IgG1, IgG2, IgG3, IgG4, IgA1, IgA2, etc. are well characterized and are known to confer functional specialization. Modified versions of each of these immunoglobulins are readily discernible to the skilled artisan in view of the instant disclosure and, accordingly, are within the scope of this disclosure.

Light chains are classified as either kappa or lambda (κ, λ). Each heavy chain class can be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are expressed. e.g., by hybridomas, B cells or genetically engineered host cells. In the heavy chain, the amino acid sequences run from an N-terminus at the forked ends of the Y configuration to the C-terminus at the bottom of each chain. The basic structure of certain antibodies, e.g., IgG antibodies, includes two heavy chain subunits and two light chain subunits covalently connected via disulfide bonds to form a “Y” structure, also referred to herein as an “H2L2” structure, or a “binding unit.”

The term “binding unit” is used herein to refer to the portion of a binding molecule, e.g., an antibody, antibody-like molecule, or antibody-derived molecule, antigen-binding fragment thereof, or multimerizing fragment thereof, which corresponds to a standard “H2L2” immunoglobulin structure, i.e., two heavy chains or fragments thereof and two light chains or fragments thereof. In certain embodiments, e.g., where the binding molecule is a bivalent IgG antibody or antigen-binding fragment thereof, the terms “binding molecule” and “binding unit” are equivalent. In other embodiments, e.g., where the binding molecule is a “multimeric binding molecule.” e.g., a dimeric IgA antibody, a dimeric IgA-like antibody, a dimeric IgA-derived binding molecule, a pentameric or hexameric IgM antibody, a pentameric or hexameric IgM-like antibody, or a pentameric or hexameric IgM-derived binding molecule or any derivative thereof, the binding molecule comprises two or more “binding units.” Two in the case of an IgA dimer, or five or six in the case of an IgM pentamer or hexamer, respectively. A binding unit need not include full-length antibody heavy and light chains, but will typically be bivalent, i.e., will include two “antigen-binding domains,” as defined above. As used herein, certain binding molecules provided in this disclosure are “dimeric,” and include two bivalent binding units that include IgA constant regions or multimerizing fragments thereof. Certain binding molecules provided in this disclosure are “pentameric” or “hexameric,” and include five or six bivalent binding units that include IgM constant regions or multimerizing fragments or variants thereof. A binding molecule, e.g., an antibody or antibody-like molecule or antibody-derived binding molecule, comprising two or more, e.g., two, five, or six binding units, is referred to herein as “multimeric.”

The term “J-chain” as used herein refers to the J-chain of IgM or IgA antibodies of any animal species, any functional fragment thereof, derivative thereof, and/or variant thereof, including a mature human J-chain, the amino acid sequence of which is presented as SEQ ID NO: 41. Various J-chain variants and modified J-chain derivatives are disclosed herein. As persons of ordinary skill in the art will recognize, “a functional fragment” or “a functional variant” includes those fragments and variants that can associate with IgM heavy chain constant regions to form a pentameric IgM antibody.

The term “modified J-chain” is used herein to refer to a derivative of a J-chain polypeptide comprising a heterologous moiety, e.g., a heterologous polypeptide, e.g., an extraneous binding domain or functional domain introduced into or attached to the J-chain sequence. The introduction can be achieved by any means, including direct or indirect fusion of the heterologous polypeptide or other moiety or by attachment through a peptide or chemical linker. The term “modified human J-chain” encompasses, without limitation, a native sequence human J-chain comprising the amino acid sequence of SEQ ID NO: 41 or functional fragment thereof, or functional variant thereof, modified by the introduction of a heterologous moiety, e.g., a heterologous polypeptide, e.g., an extraneous binding domain. In certain embodiments the heterologous moiety does not interfere with efficient polymerization of IgM into a pentamer or IgA into a dimer, and binding of such polymers to a target. Exemplary modified J-chains can be found, e.g., in U.S. Pat. Nos. 9,951,134 and 10,400,038, and in U.S. Patent Application Publication Nos. US-2019-0185570 and US-2018-0265596, each of which is incorporated herein by reference in its entirety.

As used herein the term “IgM-derived binding molecule” refers collectively to native IgM antibodies, IgM-like antibodies, as well as other IgM-derived binding molecules comprising non-antibody binding and/or functional domains instead of an antibody antigen binding domain or subunit thereof, and any fragments, e.g., multimerizing fragments, variants, or derivatives thereof.

As used herein, the term “IgM-like antibody” refers generally to a variant antibody or antibody-derived binding molecule that still retains the ability to form hexamers or pentamers, e.g., in association with a J-chain. An IgM-like antibody or other IgM-derived binding molecule typically includes at least the Cμ4-tp domains of the IgM constant region but can include heavy chain constant region domains from other antibody isotypes, e.g., IgG, from the same species or from a different species. An IgM-like antibody or other IgM-derived binding molecule can likewise be an antibody fragment in which one or more constant regions are deleted, as long as the IgM-like antibody is capable of forming hexamers and/or pentamers. Thus, an IgM-like antibody or other IgM-derived binding molecule can be, e.g., a hybrid IgM/IgG antibody or can be a “multimerizing fragment” of an IgM antibody.

The terms “valency,” “bivalent,” “multivalent” and grammatical equivalents, refer to the number of binding domains, e.g., antigen-binding domains in given binding molecule, e.g., antibody, antibody-derived, or antibody-like molecule, or in a given binding unit. As such, the terms “bivalent”, “tetravalent”, and “hexavalent” in reference to a given binding molecule, e.g., an IgM antibody, IgM-like antibody, other IgM-derived binding molecule, or multimerizing fragment thereof, denote the presence of two antigen-binding domains, four antigen-binding domains, and six antigen-binding domains, respectively. A typical IgM antibody, IgM-like antibody, or other IgM-derived binding molecule, where each binding unit is bivalent, can have 10 or 12 valencies. A bivalent or multivalent binding molecule, e.g., antibody or antibody-derived molecule, can be monospecific, i.e., all of the antigen-binding domains are the same, or can be bispecific or multispecific, e.g., where two or more antigen-binding domains are different, e.g., bind to different epitopes on the same antigen, or bind to entirely different antigens.

The term “epitope” includes any molecular determinant capable of specific binding to an antigen-binding domain of an antibody, antibody-like, or antibody-derived molecule. In certain embodiments, an epitope can include chemically active surface groupings of molecules such as amino acids, sugar side chains, phosphoryl groups, or sulfonyl groups, and, in certain embodiments, can have three-dimensional structural characteristics, and or specific charge characteristics. An epitope is a region of a target that is bound by an antigen-binding domain of an antibody.

The term “target” is used in the broadest sense to include substances that can be bound by a binding molecule, e.g., antibody, antibody-like, or antibody-derived molecule. A target can be, e.g., a polypeptide, a nucleic acid, a carbohydrate, a lipid, or other molecule, or a minimal epitope on such molecule. Moreover, a “target” can, for example, be a cell, an organ, or an organism, e.g., an animal, plant, microbe, or virus, that comprises an epitope that can be bound by a binding molecule, e.g., antibody, antibody-like, or antibody-derived molecule.

Both the light and heavy chains of antibodies, antibody-like, or antibody-derived molecules are divided into regions of structural and functional homology. The terms “constant” and “variable” are used functionally. In this regard, it will be appreciated that the variable domains of both the variable light (VL) and variable heavy (VH) chain portions determine antigen recognition and specificity. Conversely, the constant region domains of the light chain (CL) and the heavy chain (e.g., CH1, CH2, CH3, or CH4) confer biological properties such as secretion, transplacental mobility, Fc receptor binding, complement binding, and the like. By convention, the numbering of the constant region domains increases as they become more distal from the antigen-binding site or amino-terminus of the antibody. The N-terminal portion is a variable region and at the C-terminal portion is a constant region; the CH3 (or CH4, e.g., in the case of IgM) and CL domains actually comprise the carboxy-terminus of the heavy and light chain, respectively.

A “full length IgM antibody heavy chain” is a polypeptide that includes, in N-terminal to C-terminal direction, an antibody heavy chain variable domain (VH), an antibody heavy chain constant domain 1 (CM1 or Cμ1), an antibody heavy chain constant domain 2 (CM2 or Cμ2), an antibody heavy chain constant domain 3 (CM3 or Cμ3), and an antibody heavy chain constant domain 4 (CM4 or Cμ4) that can include a tailpiece.

As indicated above, variable region(s) allow a binding molecule, e.g., antibody, antibody-like, or antibody-derived molecule, to selectively recognize and specifically bind epitopes on antigens. That is, the VL domain and VH domain, or subset of the complementarity determining regions (CDRs), of a binding molecule, e.g., an antibody, antibody-like, or antibody-derived molecule, combine to form the antigen-binding domain. More specifically, an antigen-binding domain can be defined by three CDRs on each of the VH and VL chains. Certain antibodies form larger structures. For example, IgA can form a molecule that includes two H2L2 binding units and a J-chain covalently connected via disulfide bonds, which can be further associated with a secretory component, and IgM can form a pentameric or hexameric molecule that includes five or six H2L2 binding units and optionally a J-chain covalently connected via disulfide bonds.

The six “complementarity determining regions” or “CDRs” present in an antibody antigen-binding domain are short, non-contiguous sequences of amino acids that are specifically positioned to form the antigen-binding domain as the antibody assumes its three-dimensional configuration in an aqueous environment. The remainder of the amino acids in the antigen-binding domain, referred to as “framework” regions, show less inter-molecular variability. The framework regions largely adopt a p-sheet conformation and the CDRs form loops which connect, and in some cases form part of, the p-sheet structure. Thus, framework regions act to form a scaffold that provides for positioning the CDRs in correct orientation by inter-chain, non-covalent interactions. The antigen-binding domain formed by the positioned CDRs defines a surface complementary to the epitope on the immunoreactive antigen. This complementary surface promotes the non-covalent binding of the antibody to its cognate epitope. The amino acids that make up the CDRs and the framework regions, respectively, can be readily identified for any given heavy or light chain variable region by one of ordinary skill in the art, since they have been defined in various different ways (see, “Sequences of Proteins of Immunological Interest.” Kabat, E., et al., U.S. Department of Health and Human Services, (1983); and Chothia and Lesk, J. Mol. Biol., 196:901-917 (1987), which are incorporated herein by reference in their entireties).

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. These particular regions have been described, for example, by Kabat et al., U.S. Dept. of Health and Human Services, “Sequences of Proteins of Immunological Interest” (1983) and by Chothia et al., J. Mol. Biol. 196:901-917 (1987), which are incorporated herein by reference. The Kabat and Chothia definitions include overlapping or subsets of amino acids when compared against each other. Nevertheless, application of either definition (or other definitions known to those of ordinary skill in the art) to refer to a CDR of an antibody or variant thereof is intended to be within the scope of the term as defined and used herein, unless otherwise indicated. The appropriate amino acids which encompass the CDRs as defined by each of the above cited references are set forth below in Table 1 as a comparison. The exact amino acid numbers which encompass a particular CDR will vary depending on the sequence and size of the CDR. Those skilled in the art can routinely determine which amino acids comprise a particular CDR given the variable region amino acid sequence of the antibody.

TABLE 1
CDR Definitions*

| | Kabat | Chothia |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

*Numbering of all CDR definitions in Table 1 is according to the numbering conventions set forth by Kabat et al. (see below).

Antibody variable domains can also be analyzed, e.g., using the IMGT information system (imgt dot_cines_dot_fr/) (IMGT)/V-Quest) to identify variable region segments, including CDRs. (See, e.g., Brochet et al., Nucl. Acids Res. 36:W503-508, 2008).

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless use of the Kabat numbering system is explicitly noted, however, consecutive numbering is used for all amino acid sequences in this disclosure.

The Kabat numbering system for the human IgM constant domain can be found in Kabat, et. al. “Tabulation and Analysis of Amino acid and nucleic acid Sequences of Precursors, V-Regions, C-Regions, J-Chain, T-Cell Receptors for Antigen, T-Cell Surface Antigens, β-2 Microglobulins, Major Histocompatibility Antigens, Thy-1, Complement, C-Reactive Protein, Thymopoietin, Integrins, Post-gamma Globulin, α-2 Macroglobulins, and Other Related Proteins,” U.S. Dept. of Health and Human Services (1991). IgM constant regions can be numbered sequentially (i.e., amino acid #1 starting with the first amino acid of the constant region, or by using the Kabat numbering scheme. A comparison of the numbering of two alleles of the human IgM constant region sequentially (presented herein as SEQ ID NO: 35 (allele IGHM*03) and SEQ ID NO: 36 (allele IGHM*04)) and by the Kabat system is set out below. The underlined amino acid residues are not accounted for in the Kabat system (“X,” double underlined below, can be serine (S) (SEQ ID NO: 35) or glycine (G) (SEQ ID NO: 36)):

Sequential (SEQ ID NO: 35 or SEQ ID NO: 36)/
KABAT numbering key for IgM heavy chain

| | |
|---|---|
| 1/127 | GSASAPTLFP LVSCENSPSD TSSVAVGCLA |
| | QDFLPDSITF SWKYKNNSDI |
| 51/176 | SSTRGFPSVL RGGKYAATSQ VLLPSKDVMQ |
| | GTDEHVVCKV QHPNGNKEKN |
| 101/226 | VPLPVIAELP PKVSVFVPPR DGFFGNPRKS |
| | KLICQATGFS PRQIQVSWLR |
| 151/274 | EGKQVGSGVT TDQVQAEAKE SGPTTYKVTS |
| | TLTIKESDWL XQSMFTCRVD |
| 201/324 | HRGLTFQQNA SSMCVPDQDT AIRVFAIPPS |
| | FASIFLTKST KLTCLVTDLT |
| 251/374 | TYDSVTISWT RQNGEAVKTH TNISESHPKA |
| | TFSAVGEASI CEDDWNSGER |
| 301/424 | FTCTVTHTDL PSPLKQTISR PKGVALHRPD |
| | VYLLPPAREQ LNLRESATIT |
| 351/474 | CLVTGFSPAD VFVQWMQRGQ PLSPEKYVTS |
| | APMPEPQAPG RYFAHSILTV |
| 401/524 | SEEEWNTGET YTCVVAHEAL PNRVTERTVD |
| | KSTGKPTLYN VSLVMSDTAG |
| 451/574 | TCY |

Binding molecules, e.g., antibodies, antibody-like, or antibody-derived molecules, antigen-binding fragments, variants, or derivatives thereof, and/or multimerizing fragments thereof include, but are not limited to, polyclonal, monoclonal, human, humanized, or chimeric antibodies, single chain antibodies, epitope-binding fragments, e.g., Fab, Fab′ and F(ab′)2, Fd. Fvs, single-chain Fvs (scFv), single-chain antibodies, disulfide-linked Fvs (sdFv), fragments comprising either a VL or VH domain, fragments produced by a Fab expression library. ScFv molecules are known in the art and are described, e.g., in U.S. Pat. No. 5,892,019.

By “specifically binds,” it is generally meant that a binding molecule, e.g., an antibody or fragment, variant, or derivative thereof binds to an epitope via its antigen-binding domain, and that the binding entails some complementarity between the antigen-binding domain and the epitope. According to this definition, a binding molecule, e.g., antibody, antibody-like, or antibody-derived molecule, is said to “specifically bind” to an epitope when it binds to that epitope, via its antigen-binding domain more readily than it would bind to a random, unrelated epitope. The term “specificity” is used herein to qualify the relative affinity by which a certain binding molecule binds to a certain epitope. For example, binding molecule “A” can be deemed to have a higher specificity for a given epitope than binding molecule “B,” or binding molecule “A” can be said to bind to epitope “C” with a higher specificity than it has for related epitope “D.”

A binding molecule, e.g., an antibody or fragment, variant, or derivative thereof disclosed herein can be said to bind a target antigen with an off rate (k(off)) of less than or equal to 5×10-2 sec-1, 10-2 sec-1, 5×10-3 sec-1, 10-3 sec-1, 5×10-4 sec-1, 10-4 sec-1, 5×10-5 sec-1, or 10-5 sec-1 5×10-6 sec-1, 10-6 sec-1, 5×10-7 sec-1 or 10-7 sec-1.

A binding molecule, e.g., an antibody or antigen-binding fragment, variant, or derivative disclosed herein can be said to bind a target antigen with an on rate (k(on)) of greater than or equal to 103 M-1 sec-1, 5×103 M-1 sec-1, 104 M-1 sec-1, 5×104 M-1 sec-1, 105 M-1 sec-1, 5×105 M-1 sec-1, 106 M-1 sec-1, or 5×106 M-1 sec-1 or 107 M-1 sec-1.

A binding molecule, e.g., an antibody or fragment, variant, or derivative thereof is said to competitively inhibit binding of a reference antibody or antigen-binding fragment to a given epitope if it preferentially binds to that epitope to the extent that it blocks, to some degree, binding of the reference antibody or antigen-binding fragment to the epitope. Competitive inhibition can be determined by any method known in the art, for example, competition ELISA assays. A binding molecule can be said to competitively inhibit binding of the reference antibody or antigen-binding fragment to a given epitope by at least 90%, at least 80%, at least 70%, at least 60%, or at least 50%.

As used herein, the term “affinity” refers to a measure of the strength of the binding of an individual epitope with one or more antigen-binding domains, e.g., of an immunoglobulin molecule. See, e.g., Harlow et al., Antibodies: A Laboratory Manual, (Cold Spring Harbor Laboratory Press, 2nd ed. 1988) at pages 27-28. As used herein, the term “avidity” refers to the overall stability of the complex between a population of antigen-binding domains and an antigen. See, e.g., Harlow at pages 29-34. Avidity is related to both the affinity of individual antigen-binding domains in the population with specific epitopes, and also the valencies of the immunoglobulins and the antigen. For example, the interaction between a bivalent monoclonal antibody and an antigen with a highly repeating epitope structure, such as a polymer, would be one of high avidity. An interaction between a bivalent monoclonal antibody with a receptor present at a high density on a cell surface would also be of high avidity.

Binding molecules, e.g., antibodies or fragments, variants, or derivatives thereof as disclosed herein can also be described or specified in terms of their cross-reactivity. As used herein, the term “cross-reactivity” refers to the ability of a binding molecule, e.g., an antibody or fragment, variant, or derivative thereof, specific for one antigen, to react with a second antigen, a measure of relatedness between two different antigenic substances. Thus, a binding molecule is cross reactive if it binds to an epitope other than the one that induced its formation. The cross-reactive epitope generally contains many of the same complementary structural features as the inducing epitope, and in some cases, can actually fit better than the original.

A binding molecule, e.g., an antibody or fragment, variant, or derivative thereof can also be described or specified in terms of their binding affinity to an antigen. For example, a binding molecule can bind to an antigen with a dissociation constant or KD no greater than 5×10-2 M, 10-2 M, 5×10-3 M, 10-3 M, 5×10-4 M, 10-4 M, 5×10-5 M, 10-5 M, 5×10-6 M, 10-6 M, 5×10-7 M, 10-7 M, 5×10-8 M, 10-8 M, 5×10-9 M, 10-9 M, 5×10-10 M, 10-10 M, 5×10-11 M, 10-11 M, 5×10-12 M, 10-12 M, 5×10-13 M, 10-13 M, 5×10-14 M, 10-14 M, 5×10-15 M, or 10-15 M.

“Antigen-binding antibody fragments” including single-chain antibodies or other antigen-binding domains can exist alone or in combination with one or more of the following: hinge region, CH1, CH2, CH3, or CH4 domains, J-chain, or secretory component. Also included are antigen-binding fragments that can include any combination of variable region(s) with one or more of a hinge region, CH1, CH2, CH3, or CH4 domains, a J-chain, or a secretory component. Binding molecules, e.g., antibodies, or antigen-binding fragments thereof can be from any animal origin including birds and mammals. The antibodies can be human, murine, donkey, rabbit, goat, guinea pig, camel, llama, horse, or chicken antibodies. In another embodiment, the variable region can be condricthoid in origin (e.g., from sharks). As used herein, “human” antibodies include antibodies having the amino acid sequence of a human immunoglobulin and include antibodies isolated from human immunoglobulin libraries or from animals transgenic for one or more human immunoglobulins and can in some instances express endogenous immunoglobulins and some not, as described infra and, for example in, U.S. Pat. No. 5,939,598 by Kucherlapati et al. According to embodiments of the present disclosure, an IgM antibody, IgM-like antibody, or other IgM-derived binding molecule as provided herein can include an antigen-binding fragment of an antibody, e.g., a scFv fragment, so long as the IgM antibody. IgM-like antibody, or other IgM-derived binding molecule is able to form a multimer, e.g., a hexamer or a pentamer, and an IgA antibody, IgA-like antibody, or other IgA-derived binding molecule as provided herein can include an antigen-binding fragment of an antibody, e.g., a scFv fragment, so long as the IgA antibody, IgA-like antibody, or other IgA-derived binding molecule is able to form a multimer, e.g., a dimer. As used herein such a fragment comprises a “multimerizing fragment.”

As used herein, the term “heavy chain subunit” includes amino acid sequences derived from an immunoglobulin heavy chain, a binding molecule, e.g., an antibody, antibody-like, or antibody-derived molecule comprising a heavy chain subunit can include at least one of: a VH domain, a CH1 domain, a hinge (e.g., upper, middle, and/or lower hinge region) domain, a CH2 domain, a CH3 domain, a CH4 domain, or a variant or fragment thereof. For example, a binding molecule, e.g., an antibody, antibody-like, or antibody-derived molecule, or fragment, e.g., multimerizing fragment, variant, or derivative thereof can include without limitation, in addition to a VH domain; a CH1 domain; a CH1 domain, a hinge, and a CH2 domain; a CH1 domain and a CH3 domain; a CH1 domain, a hinge, and a CH3 domain; or a CH1 domain, a hinge domain, a CH2 domain, and a CH3 domain. In certain embodiments a binding molecule, e.g., an antibody, antibody-like, or antibody-derived molecule, or fragment, e.g., multimerizing fragment, variant, or derivative thereof can include, in addition to a VH domain, a CH3 domain and a CH4 domain; or a CH3 domain, a CH4 domain, and a J-chain. Further, a binding molecule, e.g., an antibody, antibody-like, or antibody-derived molecule, for use in the disclosure can lack certain constant region portions, e.g., all or part of a CH2 domain. It will be understood by one of ordinary skill in the art that these domains (e.g., the heavy chain subunit) can be modified such that they vary in amino acid sequence from the original immunoglobulin molecule. According to embodiments of the present disclosure, an IgM antibody, IgM-like antibody, or other IgM-derived binding molecule as provided herein comprises sufficient portions of an IgM heavy chain constant region to allow the IgM antibody. IgM-like antibody, or other IgM-derived binding molecule to form a multimer, e.g., a hexamer or a pentamer. As used herein such a fragment comprises a “multimerizing fragment.”

As used herein, the term “light chain subunit” includes amino acid sequences derived from an immunoglobulin light chain. The light chain subunit includes at least a VL, and can further include a CL (e.g., Cκ or Cλ) domain.

Binding molecules, e.g., antibodies, antibody-like molecules, antibody-derived molecules, antigen-binding fragments, variants, or derivatives thereof, or multimerizing fragments thereof can be described or specified in terms of the epitope(s) or portion(s) of a target, e.g., a target antigen that they recognize or specifically bind. The portion of a target antigen that specifically interacts with the antigen-binding domain of an antibody is an “epitope,” or an “antigenic determinant.” A target antigen can comprise a single epitope or at least two epitopes, and can include any number of epitopes, depending on the size, conformation, and type of antigen.

As used herein the term “disulfide bond” includes the covalent bond formed between two sulfur atoms, e.g., in cysteine residues of a polypeptide. The amino acid cysteine comprises a thiol group that can form a disulfide bond or bridge with a second thiol group. Disulfide bonds can be “intra-chain,” i.e., linking to cysteine residues in a single polypeptide or polypeptide subunit, or can be “inter-chain,” i.e., linking two separate polypeptide subunits, e.g., an antibody heavy chain and an antibody light chain, to antibody heavy chains, or an IgM or IgA antibody heavy chain constant region and a J-chain.

As used herein, the term “chimeric antibody” refers to an antibody in which the immunoreactive region or site is obtained or derived from a first species and the constant region (which can be intact, partial or modified) is obtained from a second species. In some embodiments the target binding region or site will be from a non-human source (e.g. mouse or primate) and the constant region is human.

The terms “multispecific antibody” or “bispecific antibody” refer to an antibody, antibody-like, or antibody-derived molecule that has antigen-binding domains for two or more different epitopes within a single antibody molecule. Other binding molecules in addition to the canonical antibody structure can be constructed with two binding specificities. Epitope binding by bispecific or multispecific antibodies can be simultaneous or sequential. Triomas and hybrid hybridomas are two examples of cell lines that can secrete bispecific antibodies. Bispecific antibodies can also be constructed by recombinant means. (Ströhlein and Heiss, Future Oncol. 6:1387-94 (2010); Mabry and Snavely, IDrugs. 13:543-9 (2010)). A bispecific antibody can also be a diabody.

As used herein, the term “engineered antibody” refers to an antibody in which a variable domain, constant region, and/or J-chain is altered by at least partial replacement of one or more amino acids. In certain embodiments entire CDRs from an antibody of known specificity can be grafted into the framework regions of a heterologous antibody. Although alternate CDRs can be derived from an antibody of the same class or even subclass as the antibody from which the framework regions are derived, CDRs can also be derived from an antibody of different class, e.g., from an antibody from a different species. An engineered antibody in which one or more “donor” CDRs from a non-human antibody of known specificity are grafted into a human heavy or light chain framework region is referred to herein as a “humanized antibody.” In certain embodiments not all of the CDRs are replaced with the complete CDRs from the donor variable region and yet the antigen-binding capacity of the donor can still be transferred to the recipient variable domains. Given the explanations set forth in, e.g., U.S. Pat. Nos. 5,585,089, 5,693,761, 5,693,762, and 6,180,370, it will be well within the competence of those skilled in the art, either by carrying out routine experimentation or by trial and error testing, to obtain a functional engineered or humanized antibody.

As used herein the term “engineered” includes manipulation of nucleic acid or polypeptide molecules by synthetic means (e.g. by recombinant techniques, in vitro peptide synthesis, by enzymatic or chemical coupling of peptides, nucleic acids, or glycans, or some combination of these techniques).

As used herein, the terms “linked,” “fused” or “fusion” or other grammatical equivalents can be used interchangeably. These terms refer to the joining together of two more elements or components, by whatever means including chemical conjugation or recombinant means. An “in-frame fusion” refers to the joining of two or more polynucleotide open reading frames (ORFs) to form a continuous longer ORF, in a manner that maintains the translational reading frame of the original ORFs. Thus, a recombinant fusion protein is a single protein containing two or more segments that correspond to polypeptides encoded by the original ORFs (which segments are not normally so joined in nature.) Although the reading frame is thus made continuous throughout the fused segments, the segments can be physically or spatially separated by, for example, in-frame linker sequence. For example, polynucleotides encoding the CDRs of an immunoglobulin variable region can be fused, in-frame, but be separated by a polynucleotide encoding at least one immunoglobulin framework region or additional CDR regions, as long as the “fused” CDRs are co-translated as part of a continuous polypeptide.

In the context of polypeptides, a “linear sequence” or a “sequence” is an order of amino acids in a polypeptide in an amino to carboxyl terminal direction in which amino acids that neighbor each other in the sequence are contiguous in the primary structure of the polypeptide. A portion of a polypeptide that is “amino-terminal” or “N-terminal” to another portion of a polypeptide is that portion that comes earlier in the sequential polypeptide chain. Similarly, a portion of a polypeptide that is “carboxy-terminal” or “C-terminal” to another portion of a polypeptide is that portion that comes later in the sequential polypeptide chain. For example, in a typical antibody, the variable domain is “N-terminal” to the constant region, and the constant region is “C-terminal” to the variable domain.

The term “expression” as used herein refers to a process by which a gene produces a biochemical, for example, a polypeptide. The process includes any manifestation of the functional presence of the gene within the cell including, without limitation, gene knockdown as well as both transient expression and stable expression. It includes without limitation transcription of the gene into RNA, e.g., messenger RNA (mRNA), and the translation of such mRNA into polypeptide(s). If the final desired product is a biochemical, expression includes the creation of that biochemical and any precursors. Expression of a gene produces a “gene product.” As used herein, a gene product can be either a nucleic acid, e.g., a messenger RNA produced by transcription of a gene, or a polypeptide that is translated from a transcript. Gene products described herein further include nucleic acids with post transcriptional modifications, e.g., polyadenylation, or polypeptides with post translational modifications, e.g., methylation, glycosylation, the addition of lipids, association with other protein subunits, proteolytic cleavage, and the like.

Terms such as “treating” or “treatment” or “to treat” or “alleviating” or “to alleviate” refer to therapeutic measures that cure, slow down, lessen symptoms of, and/or halt or slow the progression of an existing diagnosed pathologic condition or disorder. Terms such as “prevent,” “prevention,” “avoid,” “deterrence” and the like refer to prophylactic or preventative measures that prevent the development of an undiagnosed targeted pathologic condition or disorder. Thus, “those in need of treatment” can include those already with the disorder and/or those prone to have the disorder.

As used herein the terms “serum half-life” or “plasma half-life” refer to the time it takes (e.g., in minutes, hours, or days) following administration for the serum or plasma concentration of a drug, e.g., a binding molecule such as an antibody, antibody-like, or antibody-derived molecule or fragment, e.g., multimerizing fragment thereof as described herein, to be reduced by 50%. Two half-lives can be described: the alpha half-life, a half-life, or t½α, which is the rate of decline in plasma concentrations due to the process of drug redistribution from the central compartment, e.g., the blood in the case of intravenous delivery, to a peripheral compartment (e.g., a tissue or organ), and the beta half-life, 0 half-life, or t½β which is the rate of decline due to the processes of excretion or metabolism.

As used herein the term “area under the plasma drug concentration-time curve” or “AUC” reflects the actual body exposure to drug after administration of a dose of the drug and is expressed in mg*h/L. This area under the curve can be measured, e.g., from time 0 (t0) to infinity (∞) and is dependent on the rate of elimination of the drug from the body and the dose administered.

As used herein, the term “mean residence time” or “MRT” refers to the average length of time the drug remains in the body.

By “subject” or “individual” or “animal” or “patient” or “mammal,” is meant any subject. In certain embodiments the subject is a mammalian subject, for whom diagnosis, prognosis, or therapy is desired. Mammalian subjects include humans, domestic animals, farm animals, and zoo, sports, or pet animals such as dogs, cats, guinea pigs, rabbits, rats, mice, horses, swine, cows, bears, and so on.

As used herein, as the term “a subject that would benefit from therapy” refers to a subset of subjects, from amongst all prospective subjects, which would benefit from administration of a given therapeutic agent, e.g., a binding molecule such as an antibody, comprising one or more antigen-binding domains. Such binding molecules, e.g., antibodies, can be used, e.g., for a diagnostic procedure and/or for treatment or prevention of a disease.

### PD-1 Binding Molecules

Provided herein are multimeric binding molecules comprising two, five, or six bivalent binding units or variants or fragments thereof, wherein each binding unit comprises two IgA or IgM heavy chain constant regions or multimerizing fragments or variants thereof, each associated with a binding domain, wherein three to twelve of the binding domains are programmed cell death protein 1 (PD-1)-binding domains that specifically and agonistically bind to PD-1. In certain embodiments, the binding molecule can activate PD-1-mediated signal transduction in a cell at a higher potency than an equivalent amount of a bivalent IgG antibody or fragment thereof comprising two of the same PD-1-binding domains, which also specifically binds to and agonizes PD-1. In some embodiments, the two, five, or six binding units are human, humanized, or chimeric immunoglobulin binding units. The provided binding molecules can be used as therapeutics or diagnostics, e.g., to treat autoimmune disorders.

In some embodiments, the multimeric binding molecules are dimeric and comprise two bivalent binding units or variants or fragments thereof. In some embodiments, the multimeric binding molecules are dimeric, comprise two bivalent binding units or variants or fragments thereof, and further comprise a J-chain or functional fragment or variant thereof as described herein. In some embodiments, the multimeric binding molecules are dimeric, comprise two bivalent binding units or variants or fragments thereof, and further comprise a J-chain or functional fragment or variant thereof as described herein, wherein each binding unit comprises two IgA heavy chain constant regions or multimerizing fragments or variants thereof.

In some embodiments, the multimeric binding molecules are pentameric and comprise five bivalent binding units or variants or fragments thereof. In some embodiments, the multimeric binding molecules are pentameric and comprise five bivalent binding units or variants or fragments thereof, and further comprise a J-chain or functional fragment or variant thereof as described herein. In some embodiments, the multimeric binding molecules are pentameric and comprise five bivalent binding units or variants or fragments thereof, and further comprise a J-chain or functional fragment or variant thereof as described herein, wherein each binding unit comprises two IgM heavy chain constant regions or multimerizing fragments or variants thereof.

In some embodiments, the multimeric binding molecules are hexameric and comprise six bivalent binding units or variants or fragments thereof. In some embodiments, the multimeric binding molecules are hexameric and comprise six bivalent binding units or variants or fragments thereof, and wherein each binding unit comprises two IgM heavy chain constant regions or multimerizing fragments or variants thereof.

In certain embodiments, heavy chain constant regions in the provided binding molecules are each associated with a binding domain, e.g., an antibody antigen-binding domain, e.g., a scFv, a VHH or the VH subunit of an antibody antigen-binding domain. The multimeric binding molecule discloses herein can comprise three to twelve binding domains that are programmed cell death protein 1 (PD-1)-binding domains that specifically and agonistically bind to PD-1. In some embodiments, the multimeric binding molecule, such as an IgA antibody, an IgA-like antibody, or an IgA-derived binding molecule comprises three or four binding domains that specifically and agonistically bind to PD-1. In some embodiments, the multimeric binding molecule, such as an IgA antibody, an IgA-like antibody, or an IgA-derived binding molecule comprises four binding domains that specifically and agonistically bind to PD-1. In some embodiments, the multimeric binding molecule, such as an IgM antibody, an IgM-like antibody, or an IgM-derived binding molecule comprises ten or twelve binding domains that specifically and agonistically bind to PD-1.

In certain embodiments, the provided multimeric binding molecule is multispecific, e.g., bispecific, trispecific, or tetraspecific, where two or more binding domains associated with the heavy chain constant regions of the binding molecule specifically bind to different targets. In certain embodiments, the binding domains of the multimeric binding molecule all specifically bind to PD-1. In certain embodiments, the binding domains of the multimeric binding molecule are identical. In such cases, the multimeric binding molecule can still be bispecific, if, for example, a binding domain with a different specificity is part of a modified J-chain as described elsewhere herein. In certain embodiments, the binding domains are antibody-derived antigen-binding domains, e.g., a scFv associated with the heavy chain constant regions or a VH subunit of an antibody binding domain associated with the heavy chain constant regions.

In certain embodiments, each binding unit comprises two heavy chains each comprising a VH situated amino terminal to the heavy chain constant region, and two immunoglobulin light chains each comprising a light chain variable domain (VL) situated amino terminal to an immunoglobulin light chain constant region, e.g., a kappa or lambda constant region. The provided VH and VL combine to form an antigen-binding domain that specifically binds to the target. In certain embodiments each antigen-binding domain of each binding molecule binds to the same target, i.e., PD-1. In certain embodiments, each antigen-binding domain of each binding molecule is identical.

In certain embodiments, the three to twelve PD-1-binding domains of the multimeric binding molecule comprise a heavy chain variable region (VH) and a light chain variable region (VL), wherein the VH and VL comprise six immunoglobulin complementarity determining regions HCDR1, HCDR2, HCDR3, LCDR1, LCDR2, and LCDR3, wherein the HCDR1, HCDR2, HCDR3, LCDR1, LCDR2, and LCDR3 comprise the CDRs of an antibody comprising the VH and VL of SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, or SEQ ID NO: 49 and SEQ ID NO: 50, respectively, or the VH of any one of SEQ ID NO: 15, SEQ ID NO: 17, SEQ ID NO: 19, SEQ ID NO: 21, SEQ ID NO: 23, or SEQ ID NO: 24 and the VL of any one of SEQ ID NO: 16, SEQ ID NO: 18, SEQ ID NO: 20, or SEQ ID NO: 22, or the CDRs of an antibody comprising the VH and VL of SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, or SEQ ID NO: 49 and SEQ ID NO: 50, respectively with one or two single amino acid substitutions in one or more of the HCDRs or LCDRs, or the VH of any one of SEQ ID NO: 15, SEQ ID NO: 17, SEQ ID NO: 19, SEQ ID NO: 21, SEQ ID NO: 23, or SEQ ID NO: 24 and the VL of any one of SEQ ID NO: 16, SEQ ID NO: 18, SEQ ID NO: 20, or SEQ ID NO: 22 with one or two single amino acid substitutions in one or more of the HCDRs or LCDRs, such as the CDRs of an antibody comprising the VH and VL of SEQ ID NO: 1 and SEQ ID NO: 2, SEQ ID NO: 3 and SEQ ID NO: 4, SEQ ID NO: 13 and SEQ ID NO: 14, and SEQ ID NO: 25 and SEQ ID NO: 26, respectively, or the CDRs of an antibody comprising the VH and VL of SEQ ID NO: 1 and SEQ ID NO: 2, SEQ ID NO: 3 and SEQ ID NO: 4, SEQ ID NO: 13 and SEQ ID NO: 14, and SEQ ID NO: 25 and SEQ ID NO: 26, respectively with one or two single amino acid substitutions in one or more of the HCDRs or LCDRs.

In certain embodiments, the three to twelve PD-1-binding domains of the multimeric binding molecule comprise an antibody VH and a VL, wherein the VH and VL comprise amino acid sequences at least 80%, at least 85%, at least 90%, at least 95% or 100% identical to SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, or SEQ ID NO: 49 and SEQ ID NO: 50, respectively, or the VH of any one of SEQ ID NO: 15, SEQ ID NO: 17, SEQ ID NO: 19, SEQ ID NO: 21, SEQ ID NO: 23, SEQ ID NO: 24 and the VL of any one of SEQ ID NO: 16, SEQ ID NO: 18, SEQ ID NO: 20, or SEQ ID NO: 22, such as the amino acid sequences at least 80%, at least 85%, at least 90%, at least 95% or 100% identical to SEQ ID NO: 1 and SEQ ID NO: 2, SEQ ID NO: 3 and SEQ ID NO: 4, SEQ ID NO: 13 and SEQ ID NO: 14, and SEQ ID NO: 25 and SEQ ID NO: 26, respectively.

In certain embodiments, the three to twelve PD-1-binding domains comprise antibody VH and VL regions comprising the amino acid sequences SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, or SEQ ID NO: 49 and SEQ ID NO: 50, respectively, or the VH of any one of SEQ ID NO: 15, SEQ ID NO: 17, SEQ ID NO: 19, SEQ ID NO: 21, SEQ ID NO: 23, SEQ ID NO: 24 and the VL of any one of SEQ ID NO: 16, SEQ ID NO: 18, SEQ ID NO: 20, or SEQ ID NO: 22, such as VH and VL regions comprising the amino acid sequences SEQ ID NO: 1 and SEQ ID NO: 2. SEQ ID NO: 3 and SEQ ID NO: 4, SEQ ID NO: 13 and SEQ ID NO: 14, or SEQ ID NO: 25 and SEQ ID NO: 26, respectively.

In certain embodiments, each binding unit of the multimeric binding molecule comprises two heavy chains and two light chains, wherein the heavy chains and light chains comprise VH and VL amino acid sequences at least 80%, at least 85%, at least 90%, at least 95% or 100% identical to SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, or SEQ ID NO: 49 and SEQ ID NO: 50, respectively, or the VH of any one of SEQ ID NO: 15, SEQ ID NO: 17, SEQ ID NO: 19, SEQ ID NO: 21, SEQ ID NO: 23, or SEQ ID NO: 24 and the VL of any one of SEQ ID NO: 16, SEQ ID NO: 18, SEQ ID NO: 20, or SEQ ID NO: 22, such as VH and VL amino acid sequences at least 80%, at least 85%, at least 90%, at least 95% or 100% identical to SEQ ID NO: 1 and SEQ ID NO: 2, SEQ ID NO: 3 and SEQ ID NO: 4, SEQ ID NO: 13 and SEQ ID NO: 14, or SEQ ID NO: 25 and SEQ ID NO: 26, respectively.

In certain embodiments, the heavy chains and light chains of the multimeric binding molecule comprise the VH and VL amino acid sequences SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, or SEQ ID NO: 49 and SEQ ID NO: 50, respectively, or the VH of any one of SEQ ID NO: 15, SEQ ID NO: 17, SEQ ID NO: 19. SEQ ID NO: 21, SEQ ID NO: 23, or SEQ ID NO: 24 and the VL of any one of SEQ ID NO: 16, SEQ ID NO: 18, SEQ ID NO: 20, or SEQ ID NO: 22, such as the VH and VL amino acid sequences SEQ ID NO: 1 and SEQ ID NO: 2, SEQ ID NO: 3 and SEQ ID NO: 4, SEQ ID NO: 13 and SEQ ID NO: 14, or SEQ ID NO: 25 and SEQ ID NO: 26, respectively.

### IgM Antibodies, IgM-Like Antibodies, Other IgM-Derived Binding Molecules

IgM is the first immunoglobulin produced by B cells in response to stimulation by antigen. Naturally-occurring IgM is naturally present at around 1.5 mg/ml in serum with a half-life of about 5 days. IgM is a pentameric or hexameric molecule and thus includes five or six binding units. An IgM binding unit typically includes two light and two heavy chains. While an IgG heavy chain constant region contains three heavy chain constant domains (CH1, CH2 and CH3), the heavy (μ) constant region of IgM additionally contains a fourth constant domain (CH4) and includes a C-terminal “tailpiece.” The human IgM constant region typically comprises the amino acid sequence SEQ ID NO: 35 (identical to, e.g., GenBank Accession Nos. pir∥S37768, CAA47708.1, and. CAA47714.1, allele IGHM*03) or SEQ ID NO: 36 (identical to, e.g., GenBank Accession No. sp|P01871.4, allele IGHM*04). The human Cμ1 region ranges from about amino acid 5 to about amino acid 102 of SEQ ID NO: 35 or SEQ ID NO: 36; the human Cμ2 region ranges from about amino acid 114 to about amino acid 205 of SEQ ID NO: 35 or SEQ ID NO: 36, the human Cμ3 region ranges from about amino acid 224 to about amino acid 319 of SEQ ID NO: 35 or SEQ ID NO: 36, the Cμ4 region ranges from about amino acid 329 to about amino acid 430 of SEQ ID NO: 35 or SEQ ID NO: 36, and the tailpiece ranges from about amino acid 431 to about amino acid 453 of SEQ ID NO: 35 or SEQ ID NO: 36.

Other forms and alleles of the human IgM constant region with minor sequence variations exist, including, without limitation, GenBank Accession Nos. CAB37838.1, and pir∥MHHU. The amino acid substitutions, insertions, and/or deletions at positions corresponding to SEQ ID NO: 35 or SEQ ID NO: 36 described and claimed elsewhere in this disclosure can likewise be incorporated into alternate human IgM sequences, as well as into IgM constant region amino acid sequences of other species.

Each IgM heavy chain constant region can be associated with a binding domain, e.g., an antigen-binding domain, e.g., a scFv or VHH, or a subunit of an antigen-binding domain, e.g., a VH region. Exemplary antigen-binding domains, e.g., binding domains that specifically and agonistically bind PD-1 are described elsewhere herein. In certain embodiments the binding domain can be a non-antibody binding domain, e.g., a receptor ectodomain, a ligand or receptor-binding fragment thereof, a cytokine or receptor-binding fragment thereof, a growth factor or receptor binding fragment thereof, a neurotransmitter or receptor binding fragment thereof, a peptide or protein hormone or receptor binding fragment thereof, an immune checkpoint modulator ligand or receptor-binding fragment thereof, or a receptor-binding fragment of an extracellular matrix protein. See, e.g., PCT Application No. PCT US2019/057702, which is incorporated herein by reference in its entirety.

Five IgM binding units can form a complex with an additional small polypeptide chain (the J-chain), or a functional fragment, variant, or derivative thereof, to form a pentameric IgM antibody or IgM-like antibody, as discussed elsewhere herein. The precursor form of the human J-chain is presented as SEQ ID NO: 40. The signal peptide extends from amino acid 1 to about amino acid 22 of SEQ ID NO: 40, and the mature human J-chain extends from about amino acid 23 to amino acid 159 of SEQ ID NO: 40. The mature human J-chain includes the amino acid sequence SEQ ID NO: 41.

Exemplary variant and modified J-chains are provided elsewhere herein. Without the J-chain, an IgM antibody or IgM-like antibody typically assembles into a hexamer, comprising up to twelve antigen-binding domains. With a J-chain, an IgM antibody or IgM-like antibody typically assembles into a pentamer, comprising up to ten antigen-binding domains, or more, if the J-chain is a modified J-chain comprising one or more heterologous polypeptides comprising additional antigen-binding domain(s). The assembly of five or six IgM binding units into a pentameric or hexameric IgM antibody or IgM-like antibody is thought to involve the Cμ4 and tailpiece domains. See, e.g., Braathen, R., et al., J. Biol. Chem. 277:42755-42762 (2002). Accordingly, a pentameric or hexameric IgM antibody provided in this disclosure typically includes at least the Cμ4 and tailpiece domains (also referred to herein collectively as Cμ4-tp). A “multimerizing fragment” of an IgM heavy chain constant region thus includes at least the Cμ4-tp domains. An IgM heavy chain constant region can additionally include a Cμ3 domain or a fragment thereof, a Cμ2 domain or a fragment thereof, a Cμ1 domain or a fragment thereof, and/or other IgM heavy chain domains. In certain embodiments, an IgM-derived binding molecule, e.g., an IgM antibody, IgM-like antibody, or other IgM-derived binding molecule as provided herein can include a complete IgM heavy (p) chain constant domain, e.g., SEQ ID NO: 35 or SEQ ID NO: 36, or a variant, derivative, or analog thereof, e.g., as provided herein.

In certain embodiments, the disclosure provides a multimeric binding molecule, e.g., pentameric or hexameric binding molecule, where the binding molecule includes ten or twelve IgM-derived heavy chains, and where the IgM-derived heavy chains comprise IgM heavy chain constant regions each associated with a binding domain that specifically binds to a target. In certain embodiments, the disclosure provides an IgM antibody. IgM-like antibody, or IgM-derived binding molecule that includes five or six bivalent binding units, where each binding unit includes two IgM or IgM-like heavy chain constant regions or multimerizing fragments or variants thereof, each associated with an antigen-binding domain or subunit thereof. In certain embodiments, the two IgM heavy chain constant regions included in each binding unit are human heavy chain constant regions. In some embodiments, the heavy chains are glycosylated. In some embodiments, the heavy chains can be mutated to affect glycosylation.

Where the IgM antibody, IgM-like antibody, or other IgM-derived binding molecule provided in this disclosure is pentameric, the IgM antibody, IgM-like antibody, or other IgM-derived binding molecule typically further include a J-chain, or functional fragment or variant thereof. In certain embodiments, the J-chain is a modified J-chain or variant thereof that further comprises one or more heterologous moieties attached to the J-chain, as described elsewhere herein. In certain embodiments, the J-chain can be mutated to affect, e.g., enhance, the serum half-life of the IgM antibody, IgM-like antibody, or other IgM-derived binding molecule provided herein, as discussed elsewhere in this disclosure. In certain embodiments the J-chain can be mutated to affect glycosylation, as discussed elsewhere in this disclosure.

An IgM heavy chain constant region can include one or more of a Cμ1 domain or fragment or variant thereof, a Cμ2 domain or fragment or variant thereof, a Cμ3 domain or fragment or variant thereof, and/or a Cμ4 domain or fragment or variant thereof, provided that the constant region can serve a desired function in the IgM antibody. IgM-like antibody, or other IgM-derived binding molecule, e.g., associate with second IgM constant region to form a binding unit with one, two, or more antigen-binding domain(s), and/or associate with other binding units (and in the case of a pentamer, a J-chain) to form a hexamer or a pentamer. In certain embodiments the two IgM heavy chain constant regions or fragments or variants thereof within an individual binding unit each comprise a Cμ4 domain or fragment or variant thereof, a tailpiece (tp) or fragment or variant thereof, or a combination of a Cμ4 domain and a TP or fragment or variant thereof. In certain embodiments the two IgM heavy chain constant regions or fragments or variants thereof within an individual binding unit each further comprise a Cμ3 domain or fragment or variant thereof, a Cμ2 domain or fragment or variant thereof, a Cμ1 domain or fragment or variant thereof, or any combination thereof.

In some embodiments, the binding units of the IgM antibody, IgM-like antibody, or other IgM-derived binding molecule comprise two light chains. In some embodiments, the binding units of the IgM antibody, IgM-like antibody, or other IgM-derived binding molecule comprise two fragments light chains. In some embodiments, the light chains are kappa light chains. In some embodiments, the light chains are lambda light chains. In some embodiments, each binding unit comprises two immunoglobulin light chains each comprising a VL situated amino terminal to an immunoglobulin light chain constant region.

### IgA Antibodies, IgA-Like Antibodies, Other IgA-Derived Binding Molecules

IgA plays a critical role in mucosal immunity and comprises about 15% of total immunoglobulin produced. IgA is a monomeric or dimeric molecule. An IgA binding unit includes two light and two heavy chains. IgA contains three heavy chain constant domains (Cα1, Cα2 and Cα3), and includes a C-terminal “tailpiece.” Human IgA has two subtypes, IgA1 and IgA2. The human IgA1 constant region typically includes the amino acid sequence SEQ ID NO: 37. The human Cα1 domain extends from about amino acid 6 to about amino acid 98 of SEQ ID NO: 37; the human IgA1 hinge region extends from about amino acid 102 to about amino acid 124 of SEQ ID NO: 37, the human Cα3 domain extends from about amino acid 228 to about amino acid 330 of SEQ ID NO: 37, and the tailpiece extends from about amino acid 331 to about amino acid 352 of SEQ ID NO: 37. The human IgA2 constant region can include the amino acid sequence SEQ ID NO: 38, SEQ ID NO: 48, or other related isoforms known to those of skill in the art. The human Cα1 domain extends from about amino acid 6 to about amino acid 98 of SEQ ID NO: 38 or SEQ ID NO: 48; the human IgA2 hinge region extends from about amino acid 102 to about amino acid 111 of SEQ ID NO: 38 or SEQ ID NO: 48, the human Cα2 domain extends from about amino acid 113 to about amino acid 206 of SEQ ID NO: 38 or SEQ ID NO: 48, the human Cα3 domain extends from about amino acid 215 to about amino acid 317 of SEQ ID NO: 38 or SEQ ID NO: 48, and the tailpiece extends from about amino acid 318 to about amino acid 340 of SEQ ID NO: 38 or SEQ ID NO: 48. SEQ ID NOS: 37 and 38 are presented below:

(human IgA1 constant region)
SEQ ID NO: 37
ASPTSPKVFPLSLCSTQPDGNVVIACLVQGFFPQEPLSVT
WSESGQGVTARNFPPSQDASGDLYTTSSQLTLPATQCLAG
KSVTCHVKHYTNPSQDVTVPCPVPSTPPTPSPSTPPTPSP
SCCHPRLSLHRPALEDLLLGSEANLTCTLTGLRDASGVTF
TWTPSSGKSAVQGPPERDLCGCYSVSSVLPGCAEPWNHGK
TFTCTAAYPESKTPLTATLSKSGNTFRPEVHLLPPPSEEL
ALNELVTLTCLARGFSPKDVLVRWLQGSQELPREKYLTWA
SRQEPSQGTTTFAVTSILRVAAEDWKKGDTFSCMVGHEAL
PLAFTQKTIDRLAGKPTHVNVSVVMAEVDGTCY
(human IgA2 constant region)
SEQ ID NO: 38
ASPTSPKVFPLSLDSTPQDGNVVVACLVQGFFPQEPLSV
TWSESGQNVTARNFPPSQDASGDLYTTSSQLTLPATQCP
DGKSVTCHVKHYTNPSQDVTVPCPVPPPPPCCHPRLSLH
RPALEDLLLGSEANLTCTLTGLRDASGATFTWTPSSGKS
AVQGPPERDLCGCYSVSSVLPGCAQPWNHGETFTCTAAH
PELKTPLTANITKSGNTFRPEVHLLPPPSEELALNELVT
LTCLARGFSPKDVLVRWLQGSQELPREKYLTWASRQEPS
QGTTTFAVTSILRVAAEDWKKGDTFSCMVGHEALPLAFT
QKTIDRMAGKPTHVNVSVVMAEVDGTCY

Two IgA binding units can form a complex with two additional polypeptide chains, the J-chain (e.g., SEQ ID NO: 41 or SEQ ID NO: 42 and the secretory component (precursor, SEQ ID NO: 39, mature: amino acids 19 to 603 of SEQ ID NO: 39) to form a secretory IgA (sIgA) antibody. The assembly of IgA binding units into a dimeric sIgA antibody is thought to involve the Cα3 and tailpiece domains (also referred to herein collectively as the Cα3-tp domain). Accordingly, a dimeric sIgA antibody provided in this disclosure typically includes IgA constant regions that include at least the Cα3 and tailpiece domains. SEQ ID NO: 39 is presented below:

SEQ ID NO: 39:
MLLFVLTCLLAVFPAISTKSPIFGPEEVNSVEGNSV
SITCYYPPTSVNRHTRKYWCRQGARGGCITLISSE
GYVSSKYAGRANLTNFPENGTFVVNIAQLSQDDSG
RYKCGIGINSRGLSFDVSLEVSQGPGLLNDTKVYT
VDLGRTVTINCPFKTENAQKRKSLYKQIGLYPVLV
IDSSGYVNPNYTGRIRLDIQGTGQLLFSVVINQLR
LSDAGQYLCQAGDDSNSNKKNADLQVLKPEPELVY
EDLRGSVTFHCALGPEVANVAKFLCRQSSGENCDV
VVNTLGKRAPAFEGRIILNPQDKDGSFSVVITGLR
KEDAGRYLCGAHSDGQLQEGSPIQAWQLFVNEEST
IPRSPTVVKGVAGGSVAVLCPYNRKESKSIKYWCL
WEGAQNGRCPLLVDSEGWYKAQYEGRLSLLEEPGN
GTFTVILNQLTSRDAGFYWCLTNGDTLWRTTVEIK
IIEGEPNLKVPGNVTAVLGETLKVPCHFPCKFSSY
EKYWCKWNNTGCQALPSQDEGPSKAFVNCDENSRL
VSLTLNLVTRADEGWYWCGVKQGHFYGETAAVYVA
VEERKAAGSRDVSLAKADAAPDEKVLDSGFREIEN
KAIQDPRLFAEEKAVADTRDQADGSRASVDSGSSE
EQGGSSRALVSTLVPLGLVLAVGAVAVGVARARHR
KNVDRVSIRSYRTDISMSDFENSREFGANDNMGAS
SITQETSLGGKEEFVATTESTTETKEPKKAKRSSK
EEAEMAYKDFLLQSSTVAAEAQDGPQEA

An IgA heavy chain constant region can additionally include a Cα2 domain or a fragment thereof, an IgA hinge region, a Cα1 domain or a fragment thereof, and/or other IgA heavy chain domains. In certain aspects, an IgA antibody or IgA-like binding molecule as provided herein can include a complete IgA heavy (a) chain constant domain (e.g., SEQ ID NO: 37 or SEQ ID NO: 38), or a variant, derivative, or analog thereof. In some embodiments, the IgA heavy chain constant regions or multimerizing fragments thereof are human IgA constant regions.

In some embodiments, the binding units of the IgA antibody, IgA-like antibody, or other IgA-derived binding molecule comprise two light chains. In some embodiments, the binding units of the IgA antibody, IgA-like antibody, or other IgA-derived binding molecule comprise two fragments light chains. In some embodiments, the light chains are kappa light chains. In some embodiments, the light chains are lambda light chains. In some embodiments, each binding unit comprises two immunoglobulin light chains each comprising a VL situated amino terminal to an immunoglobulin light chain constant region.

### J-Chains and Functional Fragments or Variants Thereof

In certain embodiments, the multimeric binding molecule provided herein comprises a J-chain or functional fragment or variant thereof. In certain embodiments, the multimeric binding molecule provided herein is pentameric and comprises a J-chain or functional fragment or variant thereof. In certain embodiments, the multimeric binding molecule provided herein is dimeric and comprises a J-chain or functional fragment or variant thereof. In some embodiments, the multimeric binding molecule can comprise a naturally occurring J-chain sequence, such as a mature human J-chain sequence (e.g., SEQ ID NO: 41). Alternatively, in some embodiments, the multimeric binding molecule can comprise a variant J-chain sequence, such as a variant sequence described herein with reduced glycosylation or reduced binding to polymeric Ig receptor (e.g., pIgR). In some embodiments, the multimeric binding molecule can comprise a functional fragment of a naturally occurring or variant J-chain. As persons of ordinary skill in the art will recognize, “a functional fragment” or a “functional variant” in this context includes those fragments and variants that can associate with binding units, e.g., IgM or IgA heavy chain constant regions, to form a pentameric IgM antibody, IgM-like antibody, or IgM-derived binding molecule or a dimeric IgA antibody, IgA-like antibody, or IgA-derived binding molecule, and/or can associate with certain immunoglobulin receptors, e.g., pIgR.

In certain embodiments, the J-chain can be modified, e.g., by introduction of a heterologous moiety, or two or more heterologous moieties, e.g., polypeptides, without interfering with the ability of binding molecule to assemble and bind to its binding target(s). See U.S. Pat. Nos. 9,951,134 and 10,400.038, and U.S. Patent Application Publication Nos. US-2019-0185570 and US-2018-0265596, each of which is incorporated herein by reference in its entirety.

Accordingly, a binding molecule provided by this disclosure, including multispecific IgA, IgA-like, IgM, or IgM-like antibodies as described elsewhere herein, can comprise a modified J-chain or functional fragment or variant thereof comprising a heterologous moiety, e.g., a heterologous polypeptide, introduced, e.g., fused or chemically conjugated, into the J-chain or fragment or variant thereof. In certain embodiments, the heterologous polypeptide can be fused to the N-terminus of the J-chain or functional fragment or variant thereof, the C-terminus of the J-chain or functional fragment or variant thereof, or to both the N-terminus and C-terminus of the J-chain or functional fragment or variant thereof. In certain embodiments the heterologous polypeptide can be fused internally within the J-chain or functional fragment or variant thereof. In some embodiments, the heterologous polypeptide can be introduced into the J-chain at or near a glycosylation site. In some embodiments, the heterologous polypeptide can be introduced into the J-chain within about 10 amino acid residues from the C-terminus, or within about 10 amino acids from the N-terminus. In certain embodiments, the heterologous polypeptide can be introduced into the mature human J-chain of SEQ ID NO: 41 between cysteine residues 92 and 101 of SEQ ID NO: 41, or an equivalent location in a J-chain sequence, e.g., a J-chain variant or functional fragment of a J-chain. In a further embodiment, the heterologous polypeptide can be introduced into the mature human J-chain of SEQ ID NO: 41 at or near a glycosylation site. In a further embodiment, the heterologous polypeptide can be introduced into the mature human J-chain of SEQ ID NO: 41 within about 10 amino acid residues from the C-terminus, or within about 10 amino acids from the N-terminus.

In certain embodiments the heterologous moiety can be a peptide or polypeptide sequence fused in frame to the J-chain or chemically conjugated to the J-chain or fragment or variant thereof. In certain embodiments, the heterologous polypeptide is fused to the J-chain or functional fragment thereof via a peptide linker. Any suitable linker can be used, for example the peptide linker can include at least 5 amino acids, at least ten amino acids, and least 20 amino acids, at least 30 amino acids or more, and so on. In certain embodiments, the peptide linker includes least 5 amino acids, but no more than 25 amino acids. In certain embodiments the peptide linker can consist of 5 amino acids, 10 amino acids, 15 amino acids, 20 amino acids, or 25 amino acids. In certain embodiments, the peptide linker consists of GGGGS (SEQ ID NO: 43), GGGGSGGGGS (SEQ ID NO: 44), GGGGSGGGGSGGGGS (SEQ ID NO: 45), GGGGSGGGGSGGGGSGGGGS (SEQ ID NO: 46), or GGGGSGGGGSGGGGSGGGGSGGGGS (SEQ ID NO: 47).

In certain embodiments the heterologous moiety can be a chemical moiety conjugated to the J-chain. Heterologous moieties to be attached to a J-chain can include, without limitation, a binding moiety, e.g., an antibody or antigen-binding fragment thereof, e.g., a single chain Fv (scFv) molecule, a cytokine, e.g., IL-2 or IL-15 (see, e.g., PCT Application No. PCT US2019/057702, which is incorporated herein by reference in its entirety), a stabilizing peptide that can increase the half-life of the binding molecule, e.g., human serum albumin (HSA) or an HSA binding molecule, or a heterologous chemical moiety such as a polymer.

In some embodiments, a modified J-chain can comprise an antigen-binding domain that can include without limitation a polypeptide capable of specifically binding to a target antigen. In certain embodiments, an antigen-binding domain associated with a modified J-chain can be an antibody or an antigen-binding fragment thereof. In certain embodiments the antigen-binding domain can be a scFv antigen-binding domain or a single-chain antigen-binding domain derived, e.g., from a camelid or condricthoid antibody. In certain embodiments, the target is a target epitope, a target antigen, a target cell, or a target organ. Targets can include, without limitation, auto-immune targets, immune checkpoint inhibitors, target antigens involved in blood-brain-barrier transport, target antigens involved in neurodegenerative diseases and neuroinflammatory diseases, and any combination thereof. In certain embodiments, the binding domain, e.g., scFv fragment can bind to an effector cell. e.g., a T cell or an NK cell. In certain embodiments, the binding domain, e.g., scFv fragment can specifically bind to CD3 on cytotoxic T cells, e.g., to CD3ε. In some embodiments, the antigen binding domain binds ICOS Ligand (ICOSLG), e.g., UniProtKB-O75144; ICOS (CD278), e.g., UniProtKB-Q9Y6W8; Interleukin 6 (IL6), e.g., UniProtKB-P05231; CD28, e.g., UniProtKB-P10747; CD3, e.g., CD3ε or UniProtKB-P07766; CD80, e.g., UniProtKB-P33681; CD86, e.g., UniProtKB-P42081; Tumor Necrosis Factor Alpha (TNFa), e.g., UniProtKB-P01375; or Fibroblast Activation Protein (FAP), e.g., UniProtKB-Q12884.

The antigen-binding domain can be introduced into the J-chain at any location that allows the binding of the antigen-binding domain to its binding target without interfering with J-chain function or the function of an associated multimeric binding molecule, e.g., a pentameric IgM or dimeric IgA antibody. Insertion locations include but are not limited to at or near the C-terminus, at or near the N-terminus or at an internal location that, based on the three-dimensional structure of the J-chain, is accessible.

Variant J-Chains that Confer Increased Serum Half-Life

In certain embodiments, the J-chain is a functional variant J-chain that includes one or more single amino acid substitutions, deletions, or insertions relative to a reference J-chain identical to the variant J-chain except for the one or more single amino acid substitutions, deletions, or insertions. For example, certain amino acid substitutions, deletions, or insertions can result in the IgM-derived binding molecule exhibiting an increased serum half-life upon administration to a subject animal relative to a reference IgM-derived binding molecule that is identical except for the one or more single amino acid substitutions, deletions, or insertions in the variant J-chain, and is administered using the same method to the same animal species. In certain embodiments the variant J-chain can include one, two, three, or four single amino acid substitutions, deletions, or insertions relative to the reference J-chain.

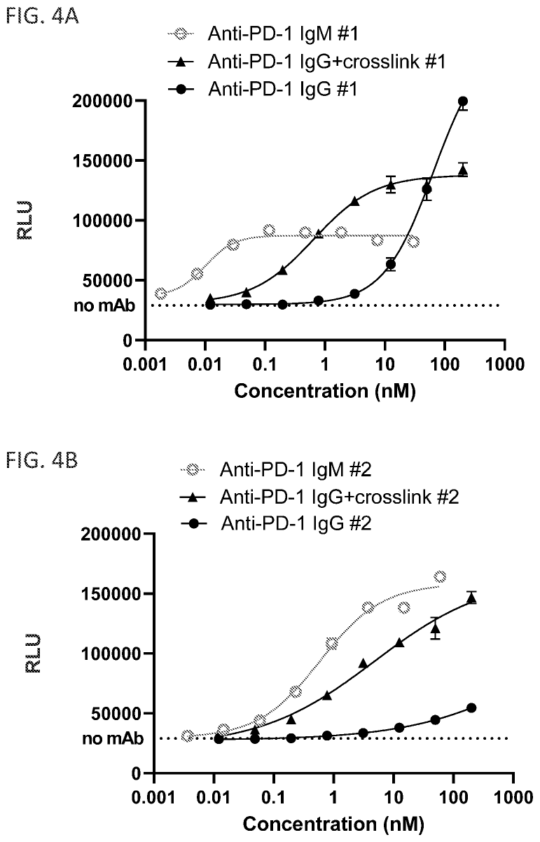

In certain embodiments, the J-chain, such as a modified J-chain, comprises an amino acid substitution at the amino acid position corresponding to amino acid Y102 of the mature wild-type human J-chain (SEQ ID NO: 41). By “an amino acid corresponding to amino acid Y102 of the mature wild-type human J-chain” is meant the amino acid in the sequence of the J-chain, which is homologous to Y102 in the human J-chain. For example, see PCT Publication No. WO 2019/169314, which is incorporated herein by reference in its entirety. The position corresponding to Y102 in SEQ ID NO: 41 is conserved in the J-chain amino acid sequences of at least 43 other species. See FIG. 4 of U.S. Pat. No. 9,951,134, which is incorporated by reference herein. Certain mutations at the position corresponding to Y102 of SEQ ID NO: 41 can inhibit the binding of certain immunoglobulin receptors, e.g., the human or murine Fcαμ receptor, the murine Fcμ receptor, and/or the human or murine polymeric Ig receptor (pIgR) to an IgM pentamer comprising the variant J-chain.

A multimeric binding molecule comprising a mutation at the amino acid corresponding to Y102 of SEQ ID NO: 41 has an improved serum half-life when administered to an animal than a corresponding multimeric binding molecule that is identical except for the substitution, and which is administered to the same species in the same manner. In certain embodiments, the amino acid corresponding to Y102 of SEQ ID NO: 41 can be substituted with any amino acid. In certain embodiments, the amino acid corresponding to Y102 of SEQ ID NO: 41 can be substituted with alanine (A), serine (S) or arginine (R). In a particular embodiment, the amino acid corresponding to Y102 of SEQ ID NO: 41 can be substituted with alanine. In a particular embodiment the J-chain or functional fragment or variant thereof is a variant human J-chain referred to herein as “J*,” and comprises the amino acid sequence SEQ ID NO: 42.

Wild-type J-chains typically include one N-linked glycosylation site. In certain embodiments, a variant J-chain or functional fragment thereof of a multimeric binding molecule as provided herein includes a mutation within the asparagine(N)-linked glycosylation motif N-X1-S/T, e.g., starting at the amino acid position corresponding to amino acid 49 (motif N6) of the mature human J-chain (SEQ ID NO: 41) or J* (SEQ ID NO: 42), wherein N is asparagine, X1 is any amino acid except proline, and SIT is serine or threonine, and wherein the mutation prevents glycosylation at that motif. As demonstrated in PCT Publication No. WO 2019/169314, mutations preventing glycosylation at this site can result in the multimeric binding molecule as provided herein, exhibiting an increased serum half-life upon administration to a subject animal relative to a reference multimeric binding molecule that is identical except for the mutation or mutations preventing glycosylation in the variant J-chain, and is administered in the same way to the same animal species.

For example, in certain embodiments the variant J-chain or functional fragment thereof of a pentameric IgM-derived or dimeric IgA-derived binding molecule as provided herein can include an amino acid substitution at the amino acid position corresponding to amino acid N49 or amino acid S51 of SEQ ID NO: 41 or SEQ ID NO: 42, provided that the amino acid corresponding to S51 is not substituted with threonine (T), or wherein the variant J-chain comprises amino acid substitutions at the amino acid positions corresponding to both amino acids N49 and S51 of SEQ ID NO: 41 or SEQ ID NO: 42. In certain embodiments, the position corresponding to N49 of SEQ ID NO: 41 or SEQ ID NO: 42 is substituted with any amino acid, e.g., alanine (A), glycine (G), threonine (T), serine (S) or aspartic acid (D). In a particular embodiment, the position corresponding to N49 of SEQ ID NO: 41 or SEQ ID NO: 42 can be substituted with alanine (A). In another particular embodiment, the position corresponding to N49 of SEQ ID NO: 41 or SEQ ID NO: 42 can be substituted with aspartic acid (D).

### Variant IgM Constant Regions

IgM heavy chain constant regions of a multimeric binding molecule as provided herein can be engineered to confer certain desirable properties to the multimeric binding molecules provided herein. For example, in certain embodiments, IgM heavy chain constant regions can be engineered to confer enhanced serum half-life to multimeric binding molecules as provided herein. Exemplary IgM heavy chain constant region mutations that can enhance serum half-life of an IgM-derived binding molecule are disclosed in PCT Publication No. WO 2019/169314, which is incorporated by reference herein in its entirety. For example, a variant IgM heavy chain constant region of the IgM antibody, IgM-like antibody, or IgM-derived binding molecule as provided herein can include an amino acid substitution at a position corresponding to amino acid S401, E402, E403, R344, and/or E345 of a wild-type human IgM constant region (e.g., SEQ ID NO: 35 or SEQ ID NO: 36). By “an amino acid corresponding to amino acid S401, E402, E403, R344, and/or E345 of a wild-type human IgM constant region” is meant the amino acid in the sequence of the IgM constant region of any species which is homologous to S401, E402, E403, R344, and/or E345 in the human IgM constant region. In certain embodiments, the amino acid corresponding to S401, E402, E403, R344, and/or E345 of SEQ ID NO: 35 or SEQ ID NO: 36 can be substituted with any amino acid, e.g., alanine.

In certain embodiments, an IgM antibody, IgM-like antibody, or other IgM-derived binding molecule as provided herein, can be engineered to exhibit reduced complement-dependent cytotoxicity (CDC) activity to cells in the presence of complement, relative to a reference IgM antibody, IgM-like antibody, or other IgM-derived binding molecule with corresponding reference human IgM constant regions identical, except for the mutations conferring reduced CDC activity. These CDC mutations can be combined with any of the mutations to confer increased serum half-life as provided herein. By “corresponding reference human IgM constant region” is meant a human IgM constant region that is identical to the variant IgM constant region except for the modification or modifications in the constant region affecting CDC activity. In certain embodiments, the variant human IgM constant region includes one or more amino acid substitutions, e.g., in the Cμ3 domain, relative to a wild-type human IgM constant region as described, e.g., in PCT Publication No. WO/2018/187702, which is incorporated herein by reference in its entirety. Assays for measuring CDC are well known to those of ordinary skill in the art, and exemplary assays are described e.g., in PCT Publication No. WO/2018/187702.

In certain embodiments, a variant human IgM constant region conferring reduced CDC activity includes an amino acid substitution corresponding to the wild-type human IgM constant region at position L310, P311, P313, and/or K315 of SEQ ID NO: 35 (human IgM constant region allele IGHM*03) or SEQ ID NO: 36 (human IgM constant region allele IGHM*04). In certain embodiments, a variant human IgM constant region conferring reduced CDC activity includes an amino acid substitution corresponding to the wild-type human IgM constant region at position P311 of SEQ ID NO: 35 or SEQ ID NO: 36. In other embodiments the variant IgM constant region as provided herein contains an amino acid substitution corresponding to the wild-type human IgM constant region at position P313 of SEQ ID NO: 35 or SEQ ID NO: 36. In other embodiments the variant IgM constant region as provided herein contains a combination of substitutions corresponding to the wild-type human IgM constant region at positions P311 of SEQ ID NO: 35 or SEQ ID NO: 36 and P313 of SEQ ID NO: 35 or SEQ ID NO: 36. These proline residues can be independently substituted with any amino acid, e.g., with alanine, serine, or glycine.

Human and certain non-human primate IgM constant regions typically include five (5) naturally-occurring asparagine (N)-linked glycosylation motifs or sites. As used herein “an N-linked glycosylation motif” comprises or consists of the amino acid sequence N-X1-S/T, wherein N is asparagine, X1 is any amino acid except proline (P), and S/T is serine (S) or threonine (T). The glycan is attached to the nitrogen atom of the asparagine residue. See, e.g., Drickamer K, Taylor M E (2006). Introduction to Glycobiology (2nd ed.). Oxford University Press, USA. N-linked glycosylation motifs occur in the human IgM heavy chain constant regions of SEQ ID NO: 35 or SEQ ID NO: 36 starting at positions 46 (“N1”), 209 (“N2”), 272 (“N3”), 279 (“N4”), and 440 (“N5”). These five motifs are conserved in non-human primate IgM heavy chain constant regions, and four of the five are conserved in the mouse IgM heavy chain constant region. Accordingly, in some embodiments, IgM heavy chain constant regions of a multimeric binding molecule as provided herein comprise 5 N-linked glycosylation motifs: N1, N2, N3, N4, and N5. In some embodiments, at least three of the N-linked glycosylation motifs (e.g., N1, N2, and N3) on each IgM heavy chain constant region are occupied by a complex glycan.

In certain embodiments, at least one, at least two, at least three, or at least four of the N-X1-S/T motifs can include an amino acid insertion, deletion, or substitution that prevents glycosylation at that motif. In certain embodiments, the IgM-derived multimeric binding molecule can include an amino acid insertion, deletion, or substitution at motif N1, motif N2, motif N3, motif N5, or any combination of two or more, three or more, or all four of motifs N1, N2, N3, or N5, where the amino acid insertion, deletion, or substitution prevents glycosylation at that motif. In some embodiment, the IgM constant region comprises two or more substitutions relative to a wild-type human IgM constant region at positions 46, 209, 272, or 440 of SEQ ID NO: 35 (human IgM constant region allele IGHM*03) or SEQ ID NO: 36 (human IgM constant region allele IGHM*04). See, e.g., U.S. Provisional Application No. 62/891,263, which is incorporated herein by reference in its entirety.

### Polynucleotides and Vectors

In certain embodiments, this disclosure provides a polynucleotide comprising a nucleic acid sequence that encodes a polypeptide subunit of a multimeric binding molecule described herein. In some embodiments, the polynucleotide encodes a polypeptide subunit comprising a heavy chain constant region and at least an antibody VH portion of the PD-1-binding domain of the multimeric binding molecule. In some embodiments, the polynucleotide encodes a polypeptide subunit comprising the heavy chain of the multimeric binding molecule.

In some embodiments, the polynucleotide encodes a polypeptide subunit comprising a light chain constant region and an antibody VL portion of the PD-1-binding domain of the multimeric binding molecule. In some embodiments, the polynucleotide encodes a polypeptide subunit comprising the light chain of the multimeric binding molecule.

In certain embodiments, this disclosure provides a vector comprising one or more polynucleotides described herein. In some embodiments, the vector further comprises a polynucleotide comprising a nucleic acid sequence that encodes a J-chain or a functional fragment or variant thereof.

In certain embodiments, this disclosure provides a composition comprising a first vector and a second vector, wherein: a) the first vector comprises a polynucleotide comprising a nucleic acid sequence that encodes the heavy chain of the multimeric binding molecule and the second vector comprises a polynucleotide comprising a nucleic acid sequence that encodes the light chain of the multimeric binding molecule, b) the first vector comprises a polynucleotide comprising a nucleic acid sequence that encodes the heavy chain of the multimeric binding molecule and a polynucleotide comprising a nucleic acid sequence that encodes the light chain of the multimeric binding molecule and the second vector comprises a polynucleotide comprising a nucleic acid sequence that encodes a J-chain or a functional fragment or variant thereof, c) the first vector comprises a polynucleotide comprising a nucleic acid sequence that encodes the heavy chain of the multimeric binding molecule and a polynucleotide comprising a nucleic acid sequence that encodes a J-chain or a functional fragment or variant thereof and the second vector comprises a polynucleotide comprising a nucleic acid sequence that encodes the light chain of the multimeric binding molecule, or d) the first vector comprises a polynucleotide comprising a nucleic acid sequence that encodes the light chain of the multimeric binding molecule and a polynucleotide comprising a nucleic acid sequence that encodes a J-chain or a functional fragment or variant thereof and the second vector comprises a polynucleotide comprising a nucleic acid sequence that encodes the heavy chain of the multimeric binding molecule. In certain embodiments, this disclosure provides a composition comprising a first vector, a second vector, and a third vector, wherein the first vector comprises a polynucleotide comprising a nucleic acid sequence that encodes the heavy chain of the multimeric binding molecule, the second vector comprises a polynucleotide comprising a nucleic acid sequence that encodes the light chain of the multimeric binding molecule, and the third vector comprises a polynucleotide comprising a nucleic acid sequence that encodes a J-chain or a functional fragment or variant thereof.

### Host Cells

In certain embodiments, this disclosure provides a host cell that is capable of producing the multimeric binding molecule as provided herein. In certain embodiments, the host cell comprises one or more vectors, a composition comprising multiple vectors, or polynucleotides disclosed herein. The disclosure also provides a method of producing the multimeric binding molecule as provided herein, where the method comprises culturing the provided host cell, and recovering the multimeric binding molecule.

### Methods of Use

The disclosure further provides a method of treating a disease or disorder in a subject in need of treatment, where the method includes administering to the subject a therapeutically effective amount of a multimeric binding molecule as provided herein. By “therapeutically effective dose or amount” or “effective amount” is intended an amount of a multimeric binding molecule that when administered brings about a positive immunotherapeutic response with respect to treatment of subject.

Effective doses of compositions for treatment of a disease or disorder vary depending upon many different factors, including means of administration, target site, physiological state of the subject, whether the subject is human or an animal, other medications administered, and whether treatment is prophylactic or therapeutic. Usually, the subject is a human, but non-human mammals including transgenic mammals can also be treated. Treatment dosages can be titrated using routine methods known to those of skill in the art to optimize safety and efficacy.

In certain embodiments, the disclosure provides a method for treating an autoimmune disorder, an inflammatory disorder, or a combination thereof in a subject in need of treatment, where the method includes administering to the subject an effective amount of a multimeric binding molecule as provided herein. In certain embodiments, administration of a multimeric binding molecule as provided herein to a subject results in greater potency than administration of an equivalent amount of a monomeric or dimeric binding molecule binding to the same binding partner. In certain embodiments the monomeric or dimeric binding molecule includes identical binding polypeptides to the multimeric binding molecule as provided herein. By “an equivalent amount” is meant, e.g., an amount measured by molecular weight, e.g., in total milligrams, or alternative, a molar equivalent, e.g., where equivalent numbers of molecules are administered.

In certain embodiments, the autoimmune disease can be, e.g., arthritis, e.g., rheumatoid arthritis, osteoarthritis, or ankylosing spondylitis, multiple sclerosis (MS), inflammatory bowel disease (IBD) e.g., Crohn's disease or ulcerative colitis, or systemic lupus erythematosus (SLE). In certain embodiments the inflammatory disease or disorder can be, e.g., arthritis, e.g., rheumatoid arthritis, or osteoarthritis, or psoriatic arthritis, Lyme disease, SLE, MS, Sjogren's syndrome, asthma, inflammatory bowel disease, ischemia, atherosclerosis, or stroke.

In other embodiments, the disclosure provides a method for preventing transplantation rejection in a transplantation recipient, where the method includes administering to the subject an effective amount of a multimeric binding molecule as provided herein. In certain embodiments, administration of a multimeric binding molecule as provided herein to a subject result in greater potency than administration of an equivalent amount of a monomeric or dimeric binding polypeptide binding to the same binding partner. In certain embodiments the monomeric or dimeric binding molecule includes identical binding polypeptides to the multimeric binding molecule as provided herein. By “an equivalent amount” is meant, e.g., an amount measured by molecular weight, e.g., in total milligrams, or alternative, a molar equivalent, e.g., where equivalent numbers of molecules are administered.

The subject to be treated can be any animal, e.g., mammal, in need of treatment, in certain embodiments, the subject is a human subject.

In its simplest form, a preparation to be administered to a subject is multimeric binding molecule as provided herein administered in a conventional dosage form, which can be combined with a pharmaceutical excipient, carrier or diluent as described elsewhere herein.

A multimeric binding molecule of the disclosure can be administered by any suitable method. e.g., parenterally, intraventricularly, orally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques.

### Pharmaceutical Compositions and Administration Methods

Methods of preparing and administering a multimeric binding molecule as provided herein to a subject in need thereof are well known to or are readily determined by those skilled in the art in view of this disclosure. The route of administration of can be, for example, oral, parenteral, by inhalation or topical. The term parenteral as used herein includes, e.g., intravenous, intraarterial, intraperitoneal, intramuscular, subcutaneous, rectal, or vaginal administration. While these forms of administration are contemplated as suitable forms, another example of a form for administration would be a solution for injection, in particular for intravenous, or intraarterial injection or drip. A suitable pharmaceutical composition can include a buffer (e.g., acetate, phosphate, or citrate buffer), a surfactant (e.g., polysorbate), optionally a stabilizer agent (e.g., human albumin), etc.

As discussed herein, a multimeric binding molecule as provided herein can be administered in a pharmaceutically effective amount for the treatment of a subject in need thereof. In this regard, it will be appreciated that the disclosed multimeric binding molecule can be formulated so as to facilitate administration and promote stability of the active agent. Pharmaceutical compositions accordingly can include a pharmaceutically acceptable, non-toxic, sterile carrier such as physiological saline, non-toxic buffers, preservatives, and the like. A pharmaceutically effective amount of a multimeric binding molecule as provided herein means an amount sufficient to achieve effective binding to a target and to achieve a therapeutic benefit. Suitable formulations are described in Remington's Pharmaceutical Sciences (Mack Publishing Co.) 16th ed. (1980).

Certain pharmaceutical compositions provided herein can be orally administered in an acceptable dosage form including, e.g., capsules, tablets, aqueous suspensions, or solutions. Certain pharmaceutical compositions also can be administered by nasal aerosol or inhalation. Such compositions can be prepared as solutions in saline, employing benzyl alcohol or other suitable preservatives, absorption promoters to enhance bioavailability, and/or other conventional solubilizing or dispersing agents.

The amount of a multimeric binding molecule that can be combined with carrier materials to produce a single dosage form will vary depending, e.g., upon the subject treated and the particular mode of administration. The composition can be administered as a single dose, multiple doses or over an established period of time in an infusion. Dosage regimens also can be adjusted to provide the optimum desired response (e.g., a therapeutic or prophylactic response).

In keeping with the scope of the present disclosure, a multimeric binding molecule as provided herein can be administered to a subject in need of therapy in an amount sufficient to produce a therapeutic effect. A multimeric binding molecule as provided herein can be administered to the subject in a conventional dosage form prepared by combining the multimeric binding molecule of the disclosure with a conventional pharmaceutically acceptable carrier or diluent according to known techniques. The form and character of the pharmaceutically acceptable carrier or diluent can be dictated by the amount of active ingredient with which it is to be combined, the route of administration and other well-known variables.

This disclosure also provides for the use of a multimeric binding molecule as provided herein in the manufacture of a medicament for treating, preventing, or managing a disease or disorder, e.g., an autoimmune disease, an inflammatory disease, or for preventing transplantation rejection.

This disclosure employs, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Green and Sambrook, ed. (2012) Molecular Cloning A Laboratory Manual (4th ed.; Cold Spring Harbor Laboratory Press); Sambrook et al., ed. (1992) Molecular Cloning: A Laboratory Manual, (Cold Springs Harbor Laboratory, NY); D. N. Glover and B. D. Hames, eds., (1995) DNA Cloning 2d Edition (IRL Press), Volumes 1-4; Gait, ed. (1990) Oligonucleotide Synthesis (IRL Press); Mullis et al. U.S. Pat. No. 4,683,195; Hames and Higgins, eds. (1985) Nucleic Acid Hybridization (IRL Press); Hames and Higgins, eds. (1984) Transcription And Translation (IRL Press); Freshney (2016) Culture Of Animal Cells, 7th Edition (Wiley-Blackwell); Woodward, J., Immobilized Cells And Enzymes (IRL Press) (1985); Perbal (1988) A Practical Guide To Molecular Cloning; 2d Edition (Wiley-Interscience); Miller and Calos eds. (1987) Gene Transfer Vectors For Mammalian Cells, (Cold Spring Harbor Laboratory); S. C. Makrides (2003) Gene Transfer and Expression in Mammalian Cells (Elsevier Science); Methods in Enzymology, Vols. 151-155 (Academic Press, Inc., N.Y.); Mayer and Walker, eds. (1987) Immunochemical Methods in Cell and Molecular Biology (Academic Press, London); Weir and Blackwell, eds.; and in Ausubel et al. (1995) Current Protocols in Molecular Biology (John Wiley and Sons).

General principles of antibody engineering are set forth, e.g., in Strohl, W. R., and L. M. Strohl (2012), Therapeutic Antibody Engineering (Woodhead Publishing). General principles of protein engineering are set forth, e.g., in Park and Cochran, eds. (2009), Protein Engineering and Design (CDC Press). General principles of immunology are set forth, e.g., in: Abbas and Lichtman (2017) Cellular and Molecular Immunology 9th Edition (Elsevier). Additionally, standard methods in immunology known in the art can be followed, e.g., in Current Protocols in Immunology (Wiley Online Library); Wild, D. (2013), The Immunoassay Handbook 4th Edition (Elsevier Science); Greenfield, ed. (2013), Antibodies, a Laboratory Manual, 2d Edition (Cold Spring Harbor Press); and Ossipow and Fischer, eds., (2014), Monoclonal Antibodies: Methods and Protocols (Humana Press).

All of the references cited above, as well as all references cited herein, are incorporated herein by reference in their entireties.

### Exemplary Embodiments

Among the provided embodiments are:

Embodiment 1. A multimeric binding molecule comprising two, five, or six bivalent binding units or variants or fragments thereof,

wherein each binding unit comprises two IgA or IgM heavy chain constant regions or multimerizing fragments or variants thereof, each associated with a binding domain,

wherein three to twelve of the binding domains are programmed cell death protein 1 (PD-1)-binding domains that specifically and agonistically bind to PD-1,

wherein the binding molecule can activate PD-1-mediated signal transduction in a cell at a higher potency than an equivalent amount of a bivalent IgG antibody or fragment thereof comprising two of the same PD-1-binding domains, which also specifically binds to and agonizes PD-1.

Embodiment 2. The multimeric binding molecule of embodiment 1, wherein the two, five, or six binding units are human, humanized, or chimeric immunoglobulin binding units.

Embodiment 3. The multimeric binding molecule of embodiment 1 or embodiment 2, wherein the three to twelve PD-1-binding domains comprise a heavy chain variable region (VH) and a light chain variable region (VL), wherein the VH and VL comprise six immunoglobulin complementarity determining regions HCDR1, HCDR2, HCDR3, LCDR1, LCDR2, and LCDR3, wherein the HCDR1, HCDR2, HCDR3, LCDR1, LCDR2, and LCDR3 comprise the CDRs of an antibody comprising the VH and VL of SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, or SEQ ID NO: 49 and SEQ ID NO: 50, respectively, or the VH of any one of SEQ ID NO: 15, SEQ ID NO: 17, SEQ ID NO: 19, SEQ ID NO: 21, SEQ ID NO: 23, or SEQ ID NO: 24 and the VL of any one of SEQ ID NO: 16, SEQ ID NO: 18, SEQ ID NO: 20, or SEQ ID NO: 22, or the CDRs of an antibody comprising the VH and VL of SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, or SEQ ID NO: 49 and SEQ ID NO: 50, respectively with one or two single amino acid substitutions in one or more of the HCDRs or LCDRs, or the VH of any one of SEQ ID NO: 15, SEQ ID NO: 17, SEQ ID NO: 19, SEQ ID NO: 21, SEQ ID NO: 23, or SEQ ID NO: 24 and the VL of any one of SEQ ID NO: 16, SEQ ID NO: 18, SEQ ID NO: 20, or SEQ ID NO: 22 with one or two single amino acid substitutions in one or more of the HCDRs or LCDRs.

Embodiment 4. The multimeric binding molecule of embodiment 3, wherein the VH and VL comprise six immunoglobulin complementarity determining regions HCDR1, HCDR2, HCDR3, LCDR1, LCDR2, and LCDR3, wherein the HCDR1, HCDR2, HCDR3, LCDR1, LCDR2, and LCDR3 comprise the CDRs of an antibody comprising the VH and VL of SEQ ID NO: 1 and SEQ ID NO: 2, SEQ ID NO: 3 and SEQ ID NO: 4, SEQ ID NO: 13 and SEQ ID NO: 14, and SEQ ID NO: 25 and SEQ ID NO: 26, respectively, or the CDRs of an antibody comprising the VH and VL of SEQ ID NO: 1 and SEQ ID NO: 2, SEQ ID NO: 3 and SEQ ID NO: 4, SEQ ID NO: 13 and SEQ ID NO: 14, and SEQ ID NO: 25 and SEQ ID NO: 26, respectively with one or two single amino acid substitutions in one or more of the HCDRs or LCDRs.

Embodiment 5. The multimeric binding molecule of any one of embodiments 1 to 3, wherein the three to twelve PD-1-binding domains of the binding molecule comprise an antibody VH and a VL, wherein the VH and VL comprise amino acid sequences at least 80%, at least 85%, at least 90%, at least 95% or 100% identical to SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, or SEQ ID NO: 49 and SEQ ID NO: 50, respectively, or the VH of any one of SEQ ID NO: 15, SEQ ID NO: 17, SEQ ID NO: 19, SEQ ID NO: 21, SEQ ID NO: 23, or SEQ ID NO: 24 and the VL of any one of SEQ ID NO: 16, SEQ ID NO: 18, SEQ ID NO: 20, or SEQ ID NO: 22.

Embodiment 6. The multimeric binding molecule of embodiment 5, wherein the three to twelve PD-1-binding domains of the binding molecule comprise an antibody VH and a VL, wherein the VH and VL comprise amino acid sequences at least 80%, at least 85%, at least 90%, at least 95% or 100% identical to SEQ ID NO: 1 and SEQ ID NO: 2, SEQ ID NO: 3 and SEQ ID NO: 4, SEQ ID NO: 13 and SEQ ID NO: 14, and SEQ ID NO: 25 and SEQ ID NO: 26, respectively.

Embodiment 7. The multimeric binding molecule of embodiment 5, wherein the three to twelve PD-1-binding domains comprise antibody VH and VL regions comprising the amino acid sequences SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, or SEQ ID NO: 49 and SEQ ID NO: 50, respectively, or the VH of any one of SEQ ID NO: 15, SEQ ID NO: 17, SEQ ID NO: 19, SEQ ID NO: 21, SEQ ID NO: 23, or SEQ ID NO: 24 and the VL of any one of SEQ ID NO: 16, SEQ ID NO: 18, SEQ ID NO: 20, or SEQ ID NO: 22.

Embodiment 8. The multimeric binding molecule of embodiment 7, wherein the three to twelve PD-1-binding domains comprise antibody VH and VL regions comprising the amino acid sequences SEQ ID NO: 1 and SEQ ID NO: 2, SEQ ID NO: 3 and SEQ ID NO: 4, SEQ ID NO: 13 and SEQ ID NO: 14, or SEQ ID NO: 25 and SEQ ID NO: 26, respectively.

Embodiment 9. The multimeric binding molecule of any one of embodiments 1 to 3, 5, or 7, wherein each binding unit comprises two heavy chains and two light chains, wherein the heavy chains and light chains comprise VH and VL amino acid sequences at least 80%, at least 85%, at least 90%, at least 95% or 100% identical to SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, or SEQ ID NO: 49 and SEQ ID NO: 50, respectively, or the VH of any one of SEQ ID NO: 15, SEQ ID NO: 17, SEQ ID NO: 19, SEQ ID NO: 21. SEQ ID NO: 23, or SEQ ID NO: 24 and the VL of any one of SEQ ID NO: 16, SEQ ID NO: 18, SEQ ID NO: 20, or SEQ ID NO: 22.

Embodiment 10. The multimeric binding molecule of embodiment 9, wherein each binding unit comprises two heavy chains and two light chains, wherein the heavy chains and light chains comprise VH and VL amino acid sequences at least 80%, at least 85%, at least 90%, at least 95% or 100% identical to SEQ ID NO: 1 and SEQ ID NO: 2, SEQ ID NO: 3 and SEQ ID NO: 4, SEQ ID NO: 13 and SEQ ID NO: 14, or SEQ ID NO: 25 and SEQ ID NO: 26, respectively.

Embodiment 11. The multimeric binding molecule of embodiment 8, wherein the heavy chains and light chains comprise the VH and VL amino acid sequences SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, or SEQ ID NO: 49 and SEQ ID NO: 50, respectively, or the VH of any one of SEQ ID NO: 15, SEQ ID NO: 17, SEQ ID NO: 19, SEQ ID NO: 21, SEQ ID NO: 23, or SEQ ID NO: 24 and the VL of any one of SEQ ID NO: 16, SEQ ID NO: 18, SEQ ID NO: 20, or SEQ ID NO: 22.

Embodiment 12. The multimeric binding molecule of embodiment 11, wherein the heavy chains and light chains comprise the VH and VL amino acid sequences SEQ ID NO: 1 and SEQ ID NO: 2, SEQ ID NO: 3 and SEQ ID NO: 4, SEQ ID NO: 13 and SEQ ID NO: 14, or SEQ ID NO: 25 and SEQ ID NO: 26, respectively.

Embodiment 13. The multimeric binding molecule of any one of embodiments 1 to 10, which is a dimeric binding molecule comprising two bivalent IgA or IgA-like binding units and a J chain or functional fragment or variant thereof, wherein each binding unit comprises two IgA heavy chain constant regions or multimerizing fragments or variants thereof, each comprising an IgA Cα3 domain and an IgA tailpiece domain.

Embodiment 14. The multimeric binding molecule of embodiment 13, wherein each IgA heavy chain constant region or multimerizing fragment or variant thereof further comprises a Cα1 domain, a Cα2 domain, an IgA hinge region, or any combination thereof.

Embodiment 15. The multimeric binding molecule of embodiment 13 or embodiment 14, wherein the IgA heavy chain constant regions or multimerizing fragments thereof are human IgA constant regions.

Embodiment 16. The multimeric binding molecule of any one of embodiments 13 to 15, wherein each binding unit comprises two IgA heavy chains each comprising a VH situated amino terminal to the IgA constant region or multimerizing fragment thereof, and two immunoglobulin light chains each comprising a VL situated amino terminal to an immunoglobulin light chain constant region.

Embodiment 17. The multimeric binding molecule of any one of embodiments 1 to 12, which is a pentameric or a hexameric binding molecule comprising five or six bivalent IgM binding units, respectively, wherein each binding unit comprises two IgM heavy chain constant regions or multimerizing fragments thereof each associated with a PD-1-binding domain, wherein each IgM heavy chain constant region comprises an IgM Cμ4 and IgM tailpiece domain.

Embodiment 18. The multimeric binding molecule of embodiment 17, wherein the IgM heavy chain constant regions or fragments or variants thereof each further comprise a Cμ1 domain, a Cμ2 domain, a Cμ3 domain, or any combination thereof.

Embodiment 19. The multimeric binding molecule of embodiment 18, wherein the IgM heavy chain constant region is a human IgM constant region.

Embodiment 20. The multimeric binding molecule of any one of embodiments 17 to 19, wherein each binding unit comprises two IgM heavy chains each comprising a VH situated amino terminal to the IgM constant region or fragment thereof, and two immunoglobulin light chains each comprising a VL situated amino terminal to an immunoglobulin light chain constant region.

Embodiment 21. The multimeric binding molecule of any one of embodiments 17 to 20, comprising SEQ ID NO: 35, SEQ ID NO: 36, or a multimerizing fragment thereof.

Embodiment 22. The multimeric binding molecule of any one of embodiments 17 to 20, wherein the IgM constant region comprises a substitution relative to a wild-type human IgM constant region at position 310, 311, 313, and/or 315 of SEQ ID NO: 35 or SEQ ID NO: 36.

Embodiment 23. The multimeric binding molecule of any one of embodiments 17 to 20, wherein the IgM constant region comprises two or more substitutions relative to a wild-type human IgM constant region at positions 46, 209, 272, or 440 of SEQ ID NO: 35 or SEQ ID NO: 36.

Embodiment 24. The multimeric binding molecule of any one of embodiments 17 to 22 which is pentameric, and further comprises a J-chain or functional fragment or variant thereof.

Embodiment 25. The multimeric binding molecule of embodiment 24, wherein the J-chain or functional fragment or variant thereof is a variant J-chain comprising one or more single amino acid substitutions, deletions, or insertions relative to a wild-type J-chain that can affect serum half-life of the multimeric binding molecule; and wherein the multimeric binding molecule comprising the variant J-chain exhibits an increased serum half-life upon administration to an animal relative to a reference multimeric binding molecule that is identical except for the one or more single amino acid substitutions, deletions, or insertions, and is administered in the same way to the same animal species.

Embodiment 26. The multimeric binding molecule of embodiment 25, wherein the J-chain or functional fragment thereof comprises an amino acid substitution at the amino acid position corresponding to amino acid Y102 of the mature wild-type human J-chain (SEQ ID NO: 41).

Embodiment 27. The multimeric binding molecule of embodiment 26, wherein the amino acid corresponding to Y102 of SEQ ID NO: 41 is substituted with alanine (A), serine (S), or arginine (R).

Embodiment 28. The multimeric binding molecule of embodiment 27, wherein the amino acid corresponding to Y102 of SEQ ID NO: 41 is substituted with alanine (A).

Embodiment 29. The multimeric binding molecule of embodiment 28, wherein the J-chain is a variant human J-chain and comprises the amino acid sequence SEQ ID NO: 42.

Embodiment 30. The multimeric binding molecule of any one of embodiments 25 to 29, wherein the J-chain or functional fragment thereof comprises an amino acid substitution at the amino acid position corresponding to amino acid N49, amino acid S51, or both N49 and S51 of the mature human J-chain (SEQ ID NO: 41), wherein a single amino acid substitution corresponding to position S51 of SEQ ID NO: 41 is not a threonine (T) substitution.

Embodiment 31. The multimeric binding molecule of embodiment 30, wherein the position corresponding to N49 of SEQ ID NO: 41 is substituted with alanine (A), glycine (G), threonine (T), serine (S) or aspartic acid (D).

Embodiment 32. The multimeric binding molecule of embodiment 31, wherein the position corresponding to N49 of SEQ ID NO: 41 or SEQ ID NO: 42 is substituted with alanine (A).

Embodiment 33. The multimeric binding molecule of any one of embodiments 30 to 32, wherein the position corresponding to S51 of SEQ ID NO: 41 or SEQ ID NO: 42 is substituted with alanine (A) or glycine (G).

Embodiment 34. The multimeric binding molecule of embodiment 33, wherein the position corresponding to S51 of SEQ ID NO: 41 or SEQ ID NO: 42 is substituted with alanine (A).

Embodiment 35. The multimeric binding molecule of any one of embodiments 13 to 16 or 24 to 34, wherein the J-chain or functional fragment or variant thereof further comprises a heterologous polypeptide, wherein the heterologous polypeptide is directly or indirectly fused to the J-chain or functional fragment or variant thereof.

Embodiment 36. The multimeric binding molecule of embodiment 35, wherein the heterologous polypeptide is fused to the J-chain or fragment thereof via a peptide linker.

Embodiment 37. The multimeric binding molecule of embodiment 36, wherein the peptide linker comprises at least 5 amino acids, but no more than 25 amino acids.

Embodiment 38. The multimeric binding molecule of embodiment 36 or 37, wherein the peptide linker consists of GGGGS (SEQ ID NO: 43), GGGGSGGGGS (SEQ ID NO: 44), GGGGSGGGGSGGGGS (SEQ ID NO: 45), GGGGSGGGGSGGGGSGGGGS (SEQ ID NO: 46), or GGGGSGGGGSGGGGSGGGGSGGGGS (SEQ ID NO: 47).

Embodiment 39. The multimeric binding molecule of any one of embodiments 35 to 38, wherein the heterologous polypeptide is fused to the N-terminus of the J-chain or fragment or variant thereof, the C-terminus of the J-chain or fragment or variant thereof, or to both the N-terminus and C-terminus of the J-chain or fragment or variant thereof.

Embodiment 40. The multimeric binding molecule of any one of embodiments 35 to 39, wherein the heterologous polypeptide can influence the absorption, distribution, metabolism and/or excretion (ADME) of the multimeric binding molecule.

Embodiment 41. The multimeric binding molecule of anyone of embodiments 35 to 39, wherein the heterologous polypeptide comprises an antigen binding domain.

Embodiment 42. The multimeric binding molecule of embodiment 41, wherein the antigen binding domain of the heterologous polypeptide is an antibody or antigen-binding fragment thereof.

Embodiment 43. The multimeric binding molecule of embodiment 42, wherein the antigen-binding fragment comprises an Fab fragment, an Fab′ fragment, an F(ab′)2 fragment, an Fd fragment, an Fv fragment, a single-chain Fv (scFv) fragment, a disulfide-linked Fv (sdFv) fragment, or any combination thereof.

Embodiment 44. The multimeric binding molecule of embodiment 42 or embodiment 43, wherein the antigen-binding fragment is a scFv fragment.

Embodiment 45. The multimeric binding molecule of any one of embodiments 41 to 44, wherein the antigen binding domain binds ICOS Ligand (ICOSLG), ICOS (CD278), Interleukin 6 (IL6), CD28, CD3, CD80, CD86, Tumor Necrosis Factor Alpha (TNFa), or Fibroblast Activation Protein (FAP).

Embodiment 46. A composition comprising the multimeric binding molecule of any one of embodiments 1 to 45.

Embodiment 47. A polynucleotide comprising a nucleic acid sequence that encodes a polypeptide subunit of the binding molecule of any one of embodiments 1 to 45.

Embodiment 48. The polynucleotide of embodiment 47, wherein the polypeptide subunit comprises an IgM heavy chain constant region and at least an antibody VH portion of the PD-1-binding domain of the multimeric binding molecule.

Embodiment 49. The polynucleotide of embodiment 48, wherein the polypeptide subunit comprises a human IgM constant region or fragment thereof fused to the C-terminal end of a VH comprising:

(a) HCDR1, HCDR2, and HCDR3 regions comprising the CDRs contained in the VH amino acid sequences SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 24, SEQ ID NO: 25, SEQ ID NO: 27, SEQ ID NO: 29, SEQ ID NO: 31, SEQ ID NO: 33, or SEQ ID NO: 49, or the CDRs contained in the VH amino acid sequences SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 24, SEQ ID NO: 25, SEQ ID NO: 27, SEQ ID NO: 29, SEQ ID NO: 31, SEQ ID NO: 33, or SEQ ID NO: 49 with one or two single amino acid substitutions in one or more of the HCDRs; or

(b) an amino acid sequence at least 80%, at least 85%, at least 90%, at least 95% or 100% identical to SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 24, SEQ ID NO: 25, SEQ ID NO: 27, SEQ ID NO: 29, SEQ ID NO: 31, SEQ ID NO: 33, or SEQ ID NO: 49.

Embodiment 50. The polynucleotide of any one of embodiments 47 to 49, wherein the polypeptide subunit comprises a light chain constant region and an antibody VL portion of the PD-1-binding domain of the multimeric binding molecule.

Embodiment 51. The polynucleotide of embodiment 50, wherein the polypeptide subunit comprises a human kappa or lambda light chain constant region or fragment thereof fused to the C-terminal end of a VL comprising:

(a) LCDR1, LCDR2, and LCDR3 regions comprising the CDRs contained in the VL amino acid sequences SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 26, SEQ ID NO: 28, SEQ ID NO: 30, SEQ ID NO: 32, SEQ ID NO: 34, or SEQ ID NO: 50, or the CDRs contained in the VL amino acid sequences SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 26, SEQ ID NO: 28, SEQ ID NO: 30, SEQ ID NO: 32, SEQ ID NO: 34, or SEQ ID NO: 50 with one or two single amino acid substitutions in one or more of the LCDRs; or

(b) an amino acid sequence at least 80%, at least 85%, at least 90%, at least 95% or 100% identical to SEQ ID NO: 2, SEQ ID NO: 4, SEQ ID NO: 6, SEQ ID NO: 8. SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 26, SEQ ID NO: 28, SEQ ID NO: 30, SEQ ID NO: 32, SEQ ID NO: 34, SEQ ID NO: 50.

Embodiment 52. A composition comprising the polynucleotide of any one of embodiments 47 to 49, and the polynucleotide of any one of embodiments 47, 50, or 51.

Embodiment 53. The composition of embodiment 52, wherein the polynucleotides are on separate vectors.

Embodiment 54. The composition of embodiment 52, wherein the polynucleotides are on a single vector.

Embodiment 55. The composition of any one of embodiments 52 to 54, further comprising a polynucleotide comprising a nucleic acid sequence encoding a J chain, or a functional fragment thereof, or a functional variant thereof.

Embodiment 56. The vector of embodiment 54.

Embodiment 57. The vectors of embodiment 53.

Embodiment 58. A host cell comprising the polynucleotide of any one of embodiments 47 to 51, the composition of any one of embodiments 52 to 55, or the vector or vectors of any one of embodiments 56 or 57, wherein the host cell can express the binding molecule of any one of embodiments 1 to 45, or a subunit thereof.

Embodiment 59. A method of producing the binding molecule of any one of embodiments 1 to 44, comprising culturing the host cell of embodiment 58, and recovering the binding molecule.

Embodiment 60. A method for treating an autoimmune disorder, an inflammatory disorder, or a combination thereof in a subject in need of treatment comprising administering to the subject an effective amount of the multimeric binding molecule of any one of embodiments 1 to 45, wherein the multimeric binding molecule exhibits greater potency than an equivalent amount of a monomeric or dimeric binding molecule binding to the same binding partner.

Embodiment 61. A method for preventing transplantation rejection in a subject, comprising administering to the subject an effective amount of the multimeric binding molecule of any one of embodiments 1 to 45, wherein the multimeric binding molecule exhibits greater potency than an equivalent amount of a monomeric or dimeric binding molecule binding to the same binding partner, and wherein the subject is a transplantation recipient.

Embodiment 62. The method of embodiment 60 or embodiment 61, wherein the subject is human.

The following examples are offered by way of illustration and not by way of limitation.

### EXAMPLES

### Example 1: Materials and Methods

The PathHunter Checkpoint Signaling Assay (Eurofins DiscoverX) is used to determine the relative level of PD-1 signaling induced by antibodies or recombinant proteins. This assay utilizes Jurkat cells, which are modified with an enzyme fragment complementation approach in which portions of the beta-galactosidase enzyme are split and covalently linked to an intracellular PD-1 signaling domain or the SHP-1 intracellular signaling mediator, which naturally associates with PD-1 during signaling events. In the presence of substrate, PD-1 signaling induces a chemiluminescent signal.

PD-1 reporter Jurkat cells are thawed and expanded according to standard cell culture procedures. Cells are seeded at 45 μL per well of a 96 well plate and antibodies are added 10× final concentration and incubated at 37 degrees Celsius for 30 minutes. PD-L1+ligand-presenting cells or additional media are added in 45 μL volume and are incubated for an additional 2-8 hours. 10 μL of Bioassay Reagent 2 is added, and cells are incubated for 15 minutes at room temperature in the dark. 40 μL of Bioassay Reagent 2 and cells are incubated for an additional 1 hour in the dark at room temperature. Chemiluminescent signal is measured on a Molecular Devices SpectraMax Paradigm and data are analyzed in GraphPad Prism.

### Example 2: Antibody Generation and Purification

### Anti-PD-1 IgM #1 and #2 and Anti-PD-1 IgG #1 and #2

As exemplary constructs, the VH and VL regions of two anti-PD-1 antibodies were incorporated into IgM (with an exemplary J-chain, SEQ ID NO: 41) and IgG formats according to standard cloning protocols. Anti-PD-1 #1 constructs include the VH and VL amino acid sequences SEQ ID NO: 13 and SEQ ID NO: 14, respectively, and Anti-PD-1 #2 constructs include the VH and VL amino acid sequences SEQ ID NO: 25 and SEQ ID NO: 26, respectively. These antibody constructs were expressed and purified according to methods described in WO2017196867. The IgM antibodies assembled as pentamers with a J-chain (data not shown).

### Example 3: PD-1 Binding by ELISA

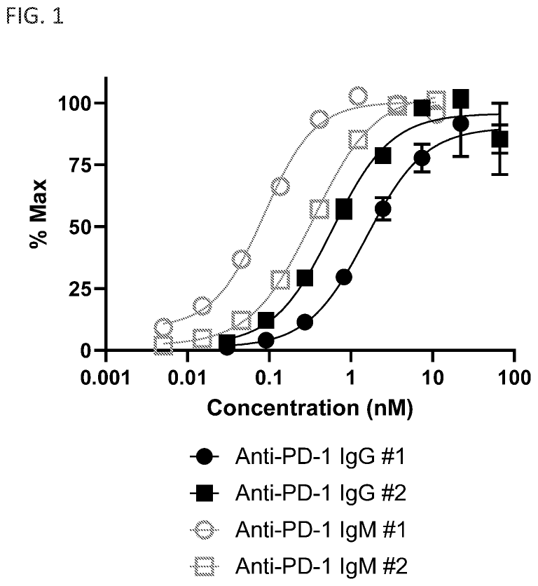

An enzyme-linked immunosorbent assay (ELISA) was performed to assess the binding of anti-PD-1 antibodies to human, cynomolgus monkey, or mouse PD-1. Recombinant proteins containing the extracellular domain of PD-1 together with either an immunoglobulin Fc region or a His tag were used to coat wells of standard 96 well ELISA plates by overnight incubation at 4° C. Unbound recombinant protein was removed, Pierce SUPERBLOCK™ T20 was added, and plates were incubated for 10 minutes at room temperature with shaking. Plates were washed with phosphate buffered saline-TWEEN-20 (PBST), and anti-PD-1 antibodies were added in phosphate buffered saline (PBS) with 1% bovine serum albumin (BSA) and incubated for one hour at room temperature with shaking. Plates were then washed in PBST and incubated with horse radish peroxidase (HRP)-conjugated secondary antibodies in F(ab′)2 directed to either human IgG or human IgM. After one hour, plates were washed again with PBST, signal was developed with SUPERSIGNAL™ ELISA Pico substrate, and an ENVISION® luminometer (PerkinElmer) was used to record signal. Data were analyzed with GRAPHPAD PRISM®. The results for human PD-1 are shown in FIG. 1. The calculated half maximal effective concentration (EC50) for each antibody and format are shown in Table 2. All antibodies also bound cynomolgus monkey PD-1 but did not bind mouse PD-1.

TABLE 2
ELISA EC₅₀

| | IgG EC₅₀ | IgM EC₅₀ |
|---|---|---|
| Antibody | (nM) | (nM) |
| #1 | 1.5 | 0.087 |
| #2 | 0.60 | 0.35 |

### Example 4: PD-1 Cell-Based Binding Assay

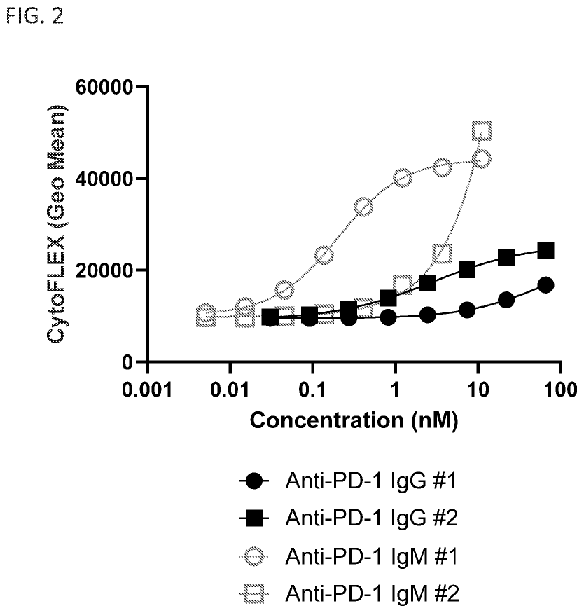

HEK293 cells were modified to overexpress human or mouse PD-1. Cells were expanded in puromycin-containing media to maintain selection, and aliquoted to 96 well V-bottom plates for antibody staining and flow cytometric analysis. Anti-PD-1 antibodies were added to cells in BD stain buffer with fetal bovine serum (BD Biosciences, cat. #554656) and incubated on ice for 20 minutes. Cells were then washed, and R-phycoerythrin (PE)-labeled anti-human IgG or anti-human IgM secondary antibodies were then added and incubated for an additional 20 minutes. Data were collected on a Beckman Coulter CYTOFLEX® cytometer and analyzed in FLOWJO™. The results for human PD-1 are shown in FIG. 2. The calculated EC50 for each antibody and format are shown in Table 3. The antibodies did not bind mouse PD-1.

TABLE 3
Cell Binding EC₅₀

| | IgG | IgM |
|---|---|---|
| Antibody | EC₅₀ (nM) | EC₅₀(nM) |
| #1 | 59 | 0.20 |
| #2 | 2.9 | n/a |

### Example 5: PD-1 Signaling Assay

The PATHHUNTER® Checkpoint Signaling Assay (Eurofins DiscoverX) was used to determine the relative level of PD-1 signaling induced by the antibodies. This assay utilizes an enzyme fragment complementation approach in which portions of the beta-galactosidase enzyme are split and covalently linked to an intracellular PD-1 signaling domain or the SHP-1 intracellular signaling mediator. When SHP-1 associates with PD-1 during signaling events, a chemiluminescent signal is generated.

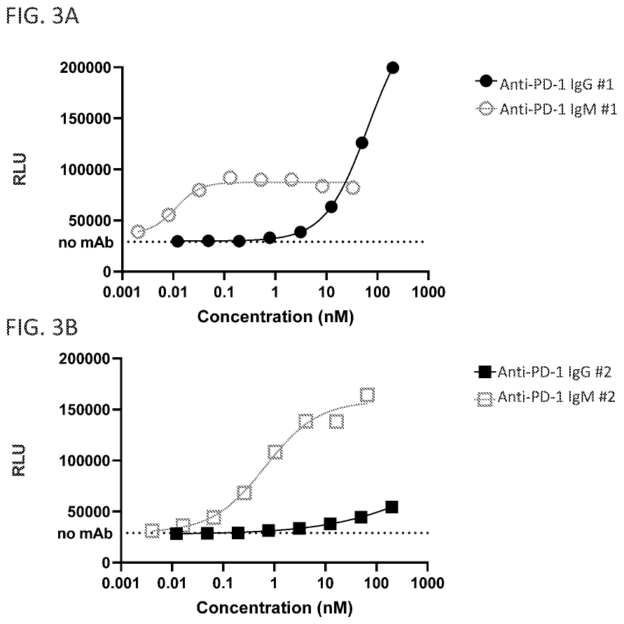

PD-1 reporter Jurkat cells were thawed and expanded according to manufacturer's instructions. Cells were seeded overnight at 40 μL per well of a 96 well plate and antibodies were added at 10× final concentration and incubated at 37° C. for 1 hour. PD-L1+ligand-presenting cells or additional media were then added in 40 μL volume and incubated for an additional 1 hour at room temperature. 10 μL of Bioassay Reagent 1 (component of Eurofins Discover X cat. #93-1104719-001117) was added, and cells were incubated for 15 minutes at room temperature in the dark. 40 μL of Bioassay Reagent 2 was added, and cells were incubated for an additional 3 hours in the dark at room temperature. Chemiluminescent signal was measured on a PerkinElmer's ENVISION® multilabel plate reader, and GRAPHPAD PRISM® was used for data analysis. The results for Antibody #1 and Antibody #2 are shown in FIGS. 3A and 3B, respectively. The calculated EC50 for each antibody and format are shown in Table 4. IgM-formatted antibodies showed increased potency over IgG-formatted antibodies.

TABLE 4
PD-1 Signaling EC₅₀

| | IgG | IgM |
|---|---|---|
| Antibody | EC₅₀ (nM) | EC₅₀(nM) |
| #1 | 65 | 0.014 |
| #2 | ND | 0.72 |

### Example 6: PD-1 Signaling Assay

The PD-1 signaling assay described in Example 5 was repeated to determine the relative level of PD-1 signaling induced by crosslinked IgG Antibody #1 and #2. The IgG antibodies were crosslinked using a “crosslinking antibody” (AffiniPure F(ab′)2 Fragment Goat Anti-Human IgG, F(ab′)2 Fragment Specific (Jackson ImmunoResearch, P/N 109-006-097)). The crosslinking antibody was diluted and added at 10 μL per well for a final crosslinking antibody to PD-1 agonist antibody ratio of 1:1.

The resulting PD-1 signaling for Antibody #1 and Antibody #2 compared to the results of Example 5 are shown in FIGS. 4A and 4B, respectively. The calculated EC50 for each antibody compared to the results of Example 5 format are shown in Table 5. IgM-formatted antibodies showed increased potency over IgG-formatted antibodies. The cross-linked IgG antibodies had an intermediate effect.

TABLE 5
PD-1 Signaling EC₅₀

| | IgG EC₅₀ | IgG/xlink EC₅₀ | IgM EC₅₀ |
|---|---|---|---|
| Antibody | (nM) | (nM) | (nM) |
| #1 | 65 | 0.65 | 0.014 |
| #2 | ND | 4.8 | 0.72 |

### Example 7: Generation and Purification of Additional Antibodies

As additional exemplary constructs, the VH and VL regions of two anti-PD-1 antibodies were incorporated into IgM (with an exemplary J-chain, SEQ ID NO: 41) and IgG formats according to standard cloning protocols. Anti-PD-1 #3 constructs include the VH and VL amino acid sequences SEQ ID NO: 1 and SEQ ID NO: 2, respectively, and Anti-PD-1 #4 constructs include the VH and VL amino acid sequences SEQ ID NO: 3 and SEQ ID NO: 4, respectively. These antibody constructs were expressed and purified according to methods described in WO2017196867. The IgM antibodies assembled as pentamers with a J-chain (data not shown).

### Example 8: PD-1 Signaling Assay

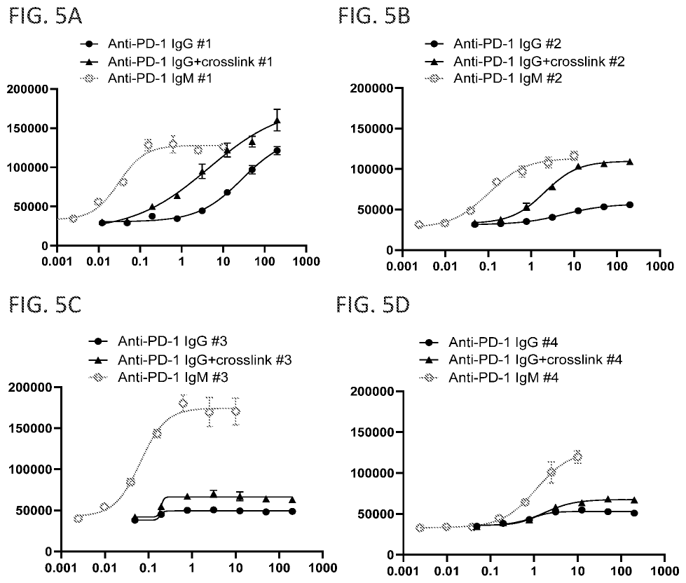

The PD-1 signaling assay was conducted according to the method in Examples 5 and 6 with the following modification: the second 1 hr incubation after the addition of additional media was conducted at 4° C. for all conditions. The results for Antibody #1-Antibody #4 are shown in FIGS. 5A-5D, respectively. Tc calculated EC50 for each antibody and format are shown in Table 6. IgM-formatted antibodies showed increased potency over IgG-formatted antibodies. The cross-linked IgG antibodies had an intermediate effect.

TABLE 6
PD-1 Signaling EC₅₀

| | IgG EC₅₀ | IgG/xlink | IgM |
|---|---|---|---|
| Antibody | (nM) | EC₅₀ (nM) | EC₅₀ (nM) |
| #1 | 29 | 4.4 | 0.031 |
| #2 | 5.9 | 2.1 | 0.10 |
| #3 | 0.19 | 0.19 | 0.065 |
| #4 | 0.89 | 1.7 | 1.1 |

### Example 9: Antibody Generation and Purification

Anti-PD-1 IgG and pentameric IgM #5

As an additional exemplary construct, the VH and VL regions of an anti-PD-1 antibody was incorporated into IgM (with an exemplary J-chain, SEQ ID NO: 41) and IgG formats according to standard cloning protocols. Anti-PD-1 #5 constructs include the VH and VL amino acid sequences SEQ ID NO: 49 and SEQ ID NO: 50, respectively. These antibody constructs were expressed and purified according to methods described in WO2017196867. The IgM antibodies assembled as pentamers with a J-chain (data not shown).

### Anti-PD-1 Hexameric IgM (IgHM) #1, #2, #3, and #5

As exemplary constructs, the VH and VL regions of three anti-PD-1 antibodies were incorporated into IgM without a J chain according to standard cloning protocols. The anti-PD-1 #1 IgHM construct included the VH and VL amino acid sequences SEQ ID NO: 13 and SEQ ID NO: 14, respectively, the anti-PD-1 #2 IgHM construct included the VH and VL amino acid sequences SEQ ID NO: 25 and SEQ ID NO: 26, respectively, the anti-PD-1 #3 IgHM construct included the VH and VL amino acid sequences SEQ ID NO: 1 and SEQ ID NO: 2, respectively, the anti-PD-1 #5 IgHM construct included the VH and VL amino acid sequences SEQ ID NO: 49 and SEQ ID NO: 50, respectively. These antibody constructs were expressed and purified according to methods described in WO2017196867. The IgM antibodies assembled as hexamers without a J-chain (data not shown).

### Example 10: PD-1 Signaling Assay

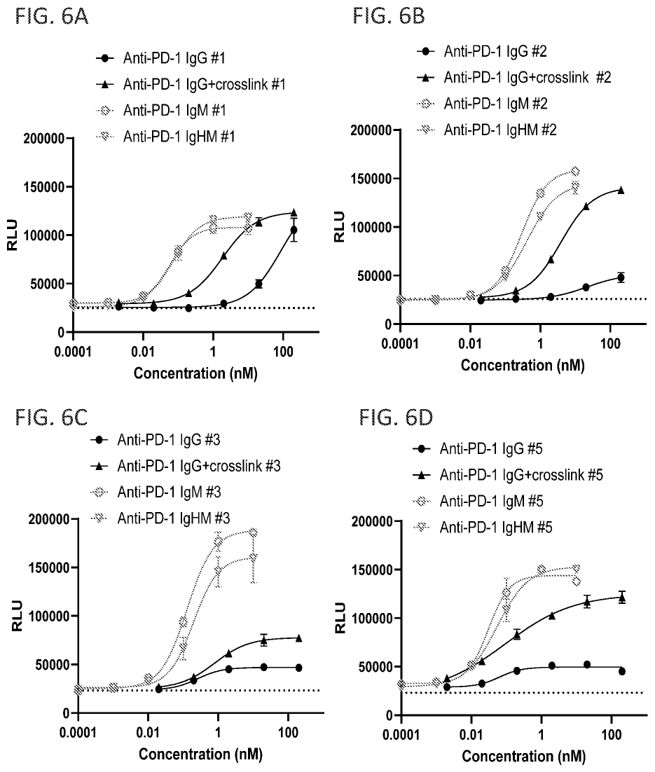

The PD-1 signaling assay was conducted according to the method in Example 8 with Antibodies #1-#3 and #5 in IgG, crosslinked IgG, pentameric IgM, and hexameric IgM (IgHM) formats. The results for Antibody #1-Antibody #3 and Antibody #5 are shown in FIGS. 6A-6D, respectively. The calculated EC50 for each antibody and format are shown in Table 7. IgM-formatted antibodies showed increased potency over IgG-formatted antibodies. The cross-linked IgG antibodies had an intermediate effect.

TABLE 7
PD-1 Signaling EC₅₀

| | IgG | IgG/xlink | IgM | IgHM |
|---|---|---|---|---|
| Antibody | EC₅₀ (nM) | EC₅₀ (nM) | EC₅₀(nM) | EC₅₀ (nM) |
| #1 | 85 | 1.8 | 0.057 | 0.070 |
| #2 | 22 | 3.8 | 0.29 | 0.40 |
| #3 | 0.26 | 0.79 | 0.13 | 0.20 |
| #5 | 0.063 | 0.093 | 0.030 | 0.054 |

TABLE 8
Anti-PD1 Antibody VH and VL Sequences

| SEQ | | SEQ | | |
|---|---|---|---|---|
| ID | VH or Heavy Chain | ID | VL or Light Chain | Reference |
| 1 | QVQLVQSGAEVKKPGASVKVSCKVSGYSLSKYDMSWVRQ | 4 | DIQMTQSPSSLSASVGDRVTITCQASQSPNNLLAWY | US10493148 B2 |
| | APGKGLEWMGIIYTSGYTDYAQKFQGRVTMTEDTSTDTA | | QQKPGKAPKLLIYGASDLPSGVPSRFSGSGSGTDFT | |
| | YMELSSLRSEDTAVYYCATGNPYYTNGFNSWGQGTLVTV | | LTISSLQPEDFATYYCQNNYYVGPVSYAFGGGTKVE | |
| | | | IK | |
| 3 | QVQLVQSGSELKKPGASVKVSCKASGYTFTDYSMHWVRQ | | EIVLTQSPATLSLSPGERATLSCTASSSVSSSYLHW | US20180355061 |
| | APGQGLEWMGWINIETGEPTYAQGFTGRFVFSLDTSVST | | YQQKPGLAPRLLIYSTSNLASGIPDRFSGSGSGTDY | A1 |
| | AYLQISSLKAEDTAVYFCARDYYGTYFYAMDYWGQGTLV | | TLTISRLEPEDFAVYYCHQYHRSPLTFGQGTKLEIK | |
| 5 | QVTLKESGPGLLQPSQTLSLTCSFSGFSLSTSGMGVSWI | 6 | DIVMTQAALSNPVTLGTSASISCRSSKSLLHSNGIT | US20180355061 |
| | RQPSGKGLEWLAHIYWDDDKRYNPSLKSRLTISKDTSSN | | YLNWYLQKPGQSPQLLIYQMSNLASGVPDRFSSSGS | A1 |
| | QVFLKITSVDTADTGTYYCVRKGYYDYGYVMDYWGQGTT | | GTDFTLRISRVEAEDVGVYYCAQNLELPLTFGSGTK | |
| | VTVSS | | LEMK | |
| 7 | QVQLQQSGAELVKPGASVKLSCKASGYTFTSYDINWVRQ | 8 | DIVLTQSPSSLSASLGERVSLTCRASQEISGYLSWL | US20180355061 |
| | RPEQGLEWIGWIFPGDGSTKYNEKFKGKATLTTDKSSST | | QQKPDGTIKRLIYAASTLDSGVPKRFSGSRSGSDYS | A1 |
| | AYMQFSRLTSEDSAVYFCARGGMRQLGRFVYWGQGTTLT | | LTISSLESEDFADYYCLQYASNPYTFGGGTKLEIK | |
| | VSS | | | |
| 9 | EVQLQQSGAELVKPGASVKLSCTASGFNVKDTYFHWVKQ | 10 | DIVMTQSPSSLSASLGDTITITCHASQNINVHLSWY | US20180355061 |
| | RPDQGLEWIGRIVSANGDTKYAPKLQDKATITTDTSSNT | | QQKPGNVPKLLIYKASNLHTGVPSRFSGSGSGTGFT | A1 |
| | AYLQLSRLTSEDTAVYYCVLIYYGFEEGDFWGQGTTLTV | | LNISSLQPEDIATYYCQQGQSFPLTFGAGTKLELK | |
| | SS | | | |
| 11 | QVQLVQSGAEVKKPGASVKVSCKASGYTFTTYWMHWVRQ | 12 | DIQMTQSPSSLSASVGDRVTITQKASQNVGTNVAWY | US20180355061 |
| | APGQGLEWMGEINPNEGGINYAQKFQGRVTLTVDKSIST | | QQKPEKAPKSLIYSASYRYSGVPSRFSGSGSGTDFT | A1 |
| | AYMELSRLRSDDTAVYYCTIDYYDYGGYWGQGTLVTVSS | | LTISSLQPEDFATYYCQQYNIYPYTFGQGTKLEIK | |
| 13 | EVQLQESGPGLVKPSQTLSLTCTVTGYSITSDYAWNWIR | 14 | DVLMTQTPLSLSVTPGQPASISCRSGQNIVHSNGNT | US8993731B2 |
| | QPPGKKLEWMGYINYSGSTSYNPSLKSRVTISRDTSKNQ | | YLEWYLQKPGQSPKLLIYKVSNRFFGVPDRISGSGS | |
| | FSLKLSSVTAADTAVYYCARWIGSSAWYFDVWGQGTLVT | | GTDFTLKISRVEAEDVGVYFCFQGSHVPFTFGQGTK | |
| | VS | | LEIK | |
| 15 | EVQLVQSGAEVKKPGASVKVSCKAFGYTFTTYPIEWMRQ | 16 | ENVLTQSPESLSASVGDRVTITCPASSSVISSYLHW | US9102728 B2 |
| | AHGQGLEWIGNFHPYNDDTKYNEKFKGRVTMTVDKSTTT | | YQQKPAKAPKLWIYSTSNLASGVPDRFSGSGSGTSY | |
| | VYMELSSLRSEDTAVYYCARENYGSHGGFVYWGQGTLVT | | TLT1SSLQPEDFATYYCQQYNSYPLTFGGGTKVEIK | |
| 17 | EVQLVQSGAEVKKPGASVKVSCKAFGYTFTTYPIEWMRQ | 18 | ENVMTQSPFSLSASVGDRVTITCRASSSVISSYLHW | US9102728 B2 |
| | AHGQGLEWIGNFHPYNDDTKYNEKFKGRVTMTRDTSTST | | YQQKPAKAPKLFIYSTSNLASGVPSRFSGSGSGTDY | |
| | VYMELSSLRSEDTAVYYCARENYGSHGGFVYWGQGTLVT | | TLTISSLQPEDFATYYCQQYNSYPLTFGGGTKVEIK | |
| | VS | | | |
| 19 | EVQLVQSGAEVKKPGASVKVSCKAFGYTFFTYPIEWVRQ | 20 | ENVLTQSPGTLSLSPGERATLSCRASSSVTSSYLHW | US9102728 B2 |
| | APGQGLEWMGNFHPYNDDTKYNEKFKGRVTMTRDTSTST | | YQQKPGQAPRLWIYSTSNLASGVPDRFSGSGSGTSY | |
| | VYMELSSLRSEDTAVYYCARENYGSHGGFVYWGQGTLVT | | TLTISRLEPEDFATYYCQQYNSYPLTFGGGTKVEIK | |
| | VS | | | |
| 21 | EVQLVQSGAEVKKPGSSVKVSCKAFGYTFTTYPIEWMRQ | 22 | ENVLTQSPFSLSASVGDRVTITCRASSSVISSYLHW | US9102728 B2 |
| | AHGQGLEWIGNFHPYNDDTKYNEKFKGRVTITVDKSTTT | | YQQKPAKAPKLFIYSTSNLASGVPSRFSGSGSGTDY | |
| | VYMELSSLRSEDTAVYYCARENYGSHGGFVYWGQGTLVT | | TLTISSLQPEDFATYYCQQYNSYPLTFGGGTKVEIK | |
| | VS | | | |
| 23 | EVQLVQSGAEVKKPGSSVKVSCKAFGYTFTTYPIEWMRQ | | | US9102728 B2 |
| | AHGQGLEWIGNFHPYNDDTKYNEKFKGRVTITADKSTST | | | |
| | AYMELSSLRSEDTAVYYCARENYGSHGGFVYWGQGTIVT | | | |
| | VS | | | |
| 24 | EVQLVQSGAEVKKPGSSVKVSCKASGYTFTTYPIEWVRQ | | | US9102728 B2 |
| | APGQGLEWMGNFHPYNDDTKYNEKFKGRVTITADKSTST | | | |
| | AYMELSSLRSEDTAVYYCARENYGSHGGFVYWGQGTLVT | | | |
| | VS | | | |
| 25 | QVQLQESGPGVVKPSGTLSLTCAISGGSIGSGGSIRSTR | 26 | NFMLTQPHSVSESPGKTVTISCTRSSGSIASNSVQW | US7488802B2 |
| | WWSWVRQSPGKGLEWIGEIYHSGSTNYNPSLKSRVTISL | | YQQRPGSSPTTVIYEDNQRPSGVPDRFSGSIDSSSN | |
| | DKSRNHFSLRLNSVTAADTAVYYCARQDYGDSGDWYFDL | | SASLTVSGLKTEDEADYYCQSSDSSAVVFGSGTKLT | |
| | WGKGTMVTVSS | | VL | |
| 27 | EVQLVQSGAEVKKPGASVKVSCKASGYRFTSYGISWVRQ | 28 | SYELTQPPSVSVSPGQTARITCSGDALPKQYAYWYQ | US7488802B2 |
| | APGQGLEWMGWISAYNGNTNYAQKLQGRVTMTTDTSTNT | | QKPGQAPVMVIYKDTERPSGIPERFSGSSSGTKVTL | |
| | AYMELRSLRSDDTAVYYCARDADYSSGSGYWGQGTLVTV | | TISGVQAEDEADYYCQSADNSITYRVFGGGTKVTVL | |
| | SS | | | |
| 29 | QVQLVQSGAEVKKPGASVRVSCKASGYTLTSYYIHWVRQ | 30 | QSALTQPASVSGSPGQSITISCTGTSNDVGGYNYVS | US7488802B2 |
| | APGQGLEWMGIINPRGATISYAQKFQGRVTMTRDTSTST | | WYQHHPGKAPKLIIYDVTNRPSGVSDRFSGSKSGNT | |
| | VYMELRNLKSEDTALYYCATAGIYGFDFDYWGRGTLVTV | | ASLTISGLLAEDEGDYYCSSYTIVTNFEVLFGGGTK | |
| | SS | | LTV | |
| 31 | QVQLQESGPGLVKPSQTLSLTCTVSGGSISSGAYYWSWI | 32 | QSVLTQPPSASGTPGQRVTISCSGSNSNIGSNSVNW | US7488802B2 |
| | RQHPGKGLEWIGYIYYNGNTYYNPSLRSLVTISVDASKN | | YQQLPGTAPKLLIYGNNQRPSGVPDRFSGSKSGTSA | |
| | QFSLKLSSVTAADTAVYYCARASDYVWGGYRYMDAFDIW | | SLAISGLQSENEADYYCAAWDDSLNGPVFGRGTKVT | |
| | GRGTLITVSS | | VLGE | |
| 33 | GAHSEVQLVQSGGGVVQPGRSLRLSCAASGFTFSSYWCD | 34 | GVHSDIVMTQSPSTLSASVGDRVTITCRASQGISSW | US7488802B2 |
| | RMSWVRQAPGKGLEWVSAISGSGGSTYYADSVKGRFT1S | | LAWYQQKPGRAPKVLIYKASTLESGVPSRFSGSGSG | |
| | RDNSKNTLYLQMNSLRAEDTAVYYCAKENWGSYFDLWGQ | | TDFTLTISSLQPEDFATYYCQQSYSTPWTFGQGTKL | |
| | GTTVTVSS | | EIKR | |
| 49 | QVQLVESGGGVVQPGRSLRLDCKASGITFSNSGMEWVRQ | 50 | EIVLTQSPATLSLSPGERATLSCRASQSVSSYLAWY | US8008449B2 |
| | APGKGLEWVAVIWYDGSKRYYADSVKGRFTISRDNSKNT | | QQKPGQAPRLLIYDASNRATGIPARFSGSGSGTDFT | |
| | LFLQMNSLRAEDTAVYYCATNDDYWGQGTLVTVSS | | LTISSLEPEDFAVYYCQQSSNWPRTFGQGTKVEIK | |

TABLE 9
Other Sequences in the Disclosure

| SEQ | Nickname | |
|---|---|---|
| ID | (source) | Sequence |
| 35 | Human IgM | GSASAPTLFPLVSCENSPSDTSSVAVGCLAQDFLPDSITFSWKYKNNSDISSTRGFPSVLRGGKYAATSQVL |
| | Constant | LPSKDVMQGTDEHVVCKVQHPNGNKEKNVPLPVIAELPPKVSVFVPPRDGFFGNPRKSKLICQATGFSPRQI |
| | region IMGT | QVSWLREGKQVGSGVTTDQVQAEAKESGPTTYKVTSTLTIKESDWLSQSMFTCRVDHRGLTFQQNASSMCVP |
| | allele | DQDTAIRVFAIPPSFASIFLTKSTKLTCLVTDLTTYDSVTISWTRQNGEAVKTHTNISESHPNATFSAVGEA |
| | IGHM*03 | SICEDDWNSGERFTCTVTHTDLPSPLKQTISRPKGVALHRPDVYLLPPAREQLNLRESATITCLVTGFSPAD |
| | (GenBank: | VFVQWMQRGQPLSPEKYVTSAPMPEPQAPGRYFAHSILTVSEEEWNTGETYTCVVAHEALPNRVTERTVDKS |
| | pir\|S37768\|) | TGKPTLYNVSLVMSDTAGTCY |
| 36 | Human IgM | GSASAPTLFPLVSCENSPSDTSSVAVGCLAQDFLPDSITFSWKYKNNSDISSTRGFPSVLRGGKYAATSQVL |
| | Constant | LPSKDVMQGTDEHVVCKVQHPNGNKEKNVPLPVIAELPPKVSVFVPPRDGFFGNPRKSKLICQATGFSPRQI |
| | region | QVSWLREGKQVGSGVTTDQVQAEAKESGPTTYKVTSTLTIKESDWLGQSMFTCRVDHRGLTFQQNASSMCVP |
| | IMGT allele | DQDTAIRVFAIPPSFASIFLTKSTKLTCLVTDLTTYDSVTISWTRQNGEAVKTHTNISESHPNATFSAVGEA |
| | IGHM*04 | SICEDDWNSGERFTCTVTHTDLPSPLKQTISRPKGVALHRPDVYLLPPAREQLNLRESATITCLVTGFSPAD |
| | (GenBank: | VFVQWMQRGQPLSPEKYVTSAPMPEPQAPGRYFAHSILTVSEEEWNTGETYTCVVAHEALPNRVTERTVDKS |
| | sp\|P01871.4\|) | TGKPTLYNVSLVMSDTAGTCY |
| 37 | Human IgA1 | ASPTSPKVFPLSLCSTQPDGNVVIACLVQGFFPQEPLSVTWSESGQGVTARNFPPSQDASGDLYTTSSQLTL |
| | heavy chain | PATQCLAGKSVTCHVKHYTNPSQDVTVPCPVPSTPPTPSPSTPPTPSPSCCHPRLSLHRPALEDLLLGSEAN |
| | constant | LTCTLTGLRDASGVTFTWTPSSGKSAVQGPPERDLCGCYSVSSVLPGCAEPWNHGKTFTCTAAYPESKTPLT |
| | region, e.g., | ATLSKSGNTFRPEVHLLPPPSEELALNELVTLTCLARGFSPKDVLVRWLQGSQELPREKYLTWASRQEPSQG |
| | amino acids | TTTFAVTSILRVAAEDWKKGDTFSCMVGHEALPLAFTQKTTDRLAGKPTHVNVSVVMAEVDGTCY |
| | 144 to 496 | |
| | of GenBank | |
| | AIC59035.1 | |
| 48 | Human IgA2 | ASPTSPKVFPLSLDSTPQDGNVVVACLVQGFFPQEPLSVTWSESGQNVTARNFPPSQDASGDLYTTSSQLTL |
| | heavy chain | PATQCPDGKSVTCHVKHYTNSSQDVTVPCRVPPPPPCCHPRLSLHRPALEDLLLGSEANLTCTLTGLRDASG |
| | constant | ATFTWTPSSGKSAVQGPPERDLCGCYSVSSVLPGCAQPWNHGETFTCTAAHPELKTPLTANITKSGNTFRPE |
| | region, e.g., | VHLLPPPSEELALNELVTLTCLARGFSPKDVLVRWLQGSQELPREKYLTWASRQEPSQGTTTYAVTSILRVA |
| | amino acids | AEDWKKGETFSCMVGHEALPLAFTQKTIDRMAGKPTHINVSVVMAEADGTCY |
| | 1 to 340 of | |
| | GenBank | |
| | P01877.4 | |
| 39 | Precursor | MLLFVLTCLLAVFPAISTKSPIFGPEEVNSVEGNSVSITCYYPPTSVNRHTRKYWCRQGARGGCITLISSEG |
| | Human | YVSSKYAGRANLTNFPENGTFVVNIAQLSQDDSGRYKCGLGINSRGLSFDVSLEVSQGPGLLNDTKVYTVDL |
| | Secretory | GRTVTINCPFKTENAQKRKSLYKQIGLYPVLVIDSSGYVNPNYTGRIRLDIQGTGQLLFSVVINQLRLSDAG |
| | Component | QYLCQAGDDSNSNKKNADLQVLKPEPELVYEDLRGSVTFHCALGPEVANVAKFLCRQSSGENCDVVVNTLGK |
| | | RAPAFEGRILLNPQDKDGSFSVVITGLRKEDAGRYLCGAHSDGQLQEGSPIQAWQLFVNEESTIPRSPTVVK |
| | | GVAGGSVAVLCPYNRKESKSIKYWCLWEGAQNGRCPLLVDSEGWVKAQYEGRLSLLEEPGNGTFTVILNQLT |
| | | SRDAGFYWCLTNGDTLWRTTVEIKIIEGEPNLKVPGNVTAVLGETLKVPCHFPCKFSSYEKYWCKWNNTGCQ |
| | | ALPSQDEGPSKAFVNCDENSRLVSLTLNLVTRADEGWYWCGVKQGHFYGETAAVYVAVEERKAAGSRDVSLA |
| | | KADAAPDEKVIDSGFREIENKAIQDPRLFAEEKAVADTRDQADGSRASVDSGSSEEQGGSSRALVSTLVPLG |
| | | LVLAVGAVAVGVARARHRKNVDRVSIRSYRTDISMSDFENSREFGANDNMGASSITQETSLGGKEEFVATTE |
| | | STTETKEPKKAKRSSKEEAEMAYKDFLLQSSTVAAEAQDGPQEA |
| 40 | Precursor | MKNHLLFWGVLAVFIKAVHVKAQEDERIVLVDNKCKCARITSRIIRSSEDPNEDIVERNIRIIVPLNNRENI |
| | Human J Chain | SDPTSPLRTRFVYHLSDLCKKCDPTEVELDNQIVTATQSNICDEDSATETCYTYDRNKCYTAVVPLVYGGET |
| | | KMVETALTPDACYPD |
| 41 | Mature Human | QEDERIVLVDNKCKCARITSRIIRSSEDPNEDIVERNIRIIVPLNNRENISDPTSPLRTRFVYHLSDLCKKC |
| | J Chain | DPTEVELDNQIVTATQSNICDEDSATETCYTYDRNKCYTAVVPLVYGGETKMVETALTPDACYPD |
| 42 | J Chain Y102A | QEDERIVLVDNKCKCARITSRIIRSSEDPNEDIVERNIRIIVPLNNRENISDPTSPLRTRFVYHLSDLCKKC |
| | mutation | DPTEVELDNQIVTATQSNICDEDSATETCATYDRNKCYTAVVPLVYGGETKMVETALTPDACYPD |
| 43 | “5” Peptide | GGGGS |
| | linker | |
| 44 | “10” Peptide | GGGGSGGGGS |
| | linker | |
| 45 | “15” Peptide | GGGGSGGGGSGGGGS |
| | linker | |
| 46 | “20” Peptide | GGGGSGGGGSGGGGSGGGGS |
| | linker | |
| 47 | “25” Peptide | GGGGSGGGGSGGGGSGGGGSGGGGS |
| | Linker | |

What is claimed is:

1. A multimeric binding molecule comprising two, five, or six bivalent binding units or variants or fragments thereof,
wherein each binding unit comprises two IgA or IgM heavy chain constant regions or multimerizing fragments or variants thereof, each associated with a binding domain,
wherein three to twelve of the binding domains are programmed cell death protein 1 (PD-1)-binding domains that specifically and agonistically bind to PD-1,
wherein the binding molecule can activate PD-1-mediated signal transduction in a cell at a higher potency than an equivalent amount of a bivalent IgG antibody or fragment thereof comprising two of the same PD-1-binding domains, which also specifically binds to and agonizes PD-1.

2. The multimeric binding molecule of claim 1, wherein the three to twelve PD-1-binding domains comprise a heavy chain variable region (VH) and a light chain variable region (VL), wherein:
(a) the VH and VL comprise six immunoglobulin complementarity determining regions HCDR1, HCDR2, HCDR3, LCDR1, LCDR2, and LCDR3, wherein the HCDR1, HCDR2, HCDR3, LCDR1, LCDR2, and LCDR3 comprise the CDRs of an antibody comprising the VH and VL of SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, or SEQ ID NO: 49 and SEQ ID NO: 50, respectively with zero, one, or two single amino acid substitutions in one or more of the HCDRs or LCDRs;
(b) the VH and VL comprise six immunoglobulin complementarity determining regions HCDR1, HCDR2, HCDR3, LCDR1, LCDR2, and LCDR3, wherein the HCDR1, HCDR2, HCDR3, LCDR1, LCDR2, and LCDR3 comprise the CDRs of an antibody comprising the VH of any one of SEQ ID NO: 15, SEQ ID NO: 17, SEQ ID NO: 19, SEQ ID NO: 21, SEQ ID NO: 23, or SEQ ID NO: 24 and the VL of any one of SEQ ID NO: 16, SEQ ID NO: 18, SEQ ID NO: 20, or SEQ ID NO: 22 with zero, one, or two single amino acid substitutions in one or more of the HCDRs or LCDRs;
(c) the VH and VL comprise amino acid sequences at least 80%, at least 85%, at least 90%, at least 95% or 100% identical to SEQ ID NO: 1 and SEQ ID NO: 2, SEQ ID NO: 3 and SEQ ID NO: 4, SEQ ID NO: 5 and SEQ ID NO: 6, SEQ ID NO: 7 and SEQ ID NO: 8, SEQ ID NO: 9 and SEQ ID NO: 10, SEQ ID NO: 11 and SEQ ID NO: 12, SEQ ID NO: 13 and SEQ ID NO: 14, SEQ ID NO: 25 and SEQ ID NO: 26, SEQ ID NO: 27 and SEQ ID NO: 28, SEQ ID NO: 29 and SEQ ID NO: 30, SEQ ID NO: 31 and SEQ ID NO: 32, SEQ ID NO: 33 and SEQ ID NO: 34, or SEQ ID NO: 49 and SEQ ID NO: 50, respectively; or
(d) the VH comprises an amino acid sequence at least 80%, at least 85%, at least 90%, at least 95% or 100% identical to of any one of SEQ ID NO: 15, SEQ ID NO: 17, SEQ ID NO: 19, SEQ ID NO: 21, SEQ ID NO: 23, or SEQ ID NO: 24 and the VL comprises an amino acid sequence at least 80%, at least 85%, at least 90%, at least 95% or 100% identical to of any one of SEQ ID NO: 16, SEQ ID NO: 18, SEQ ID NO: 20, or SEQ ID NO: 22.

3. The multimeric binding molecule of claim 2, wherein the VH and VL comprise six immunoglobulin complementarity determining regions HCDR1, HCDR2, HCDR3, LCDR1, LCDR2, and LCDR3, wherein the HCDR1, HCDR2, HCDR3, LCDR1, LCDR2, and LCDR3 comprise the CDRs of an antibody comprising the VH and VL of SEQ ID NO: 1 and SEQ ID NO: 2, SEQ ID NO: 3 and SEQ ID NO: 4, SEQ ID NO: 13 and SEQ ID NO: 14, and SEQ ID NO: 25 and SEQ ID NO: 26, respectively with zero, one, or two single amino acid substitutions in one or more of the HCDRs or LCDRs.

4. The multimeric binding molecule of claim 1, which is a dimeric binding molecule comprising two bivalent IgA or IgA-like binding units and a J chain or functional fragment or variant thereof, wherein each binding unit comprises two IgA heavy chain constant regions or multimerizing fragments or variants thereof, each comprising an IgA Cα3 domain and an IgA tailpiece domain.

5. The multimeric binding molecule of claim 4, wherein:
(a) each IgA heavy chain constant region or multimerizing fragment or variant thereof further comprises a Cα1 domain, a Cα2 domain, an IgA hinge region, or any combination thereof;
(b) the IgA heavy chain constant regions or multimerizing fragments thereof are human IgA constant regions, and/or
(c) each binding unit comprises two IgA heavy chains each comprising a VH situated amino terminal to the IgA constant region or multimerizing fragment thereof, and two immunoglobulin light chains each comprising a VL situated amino terminal to an immunoglobulin light chain constant region.

6. The multimeric binding molecule of claim 1, which is a pentameric or a hexameric binding molecule comprising five or six bivalent IgM binding units, respectively, wherein each binding unit comprises two IgM heavy chain constant regions or multimerizing fragments thereof each associated with a PD-1-binding domain, wherein each IgM heavy chain constant region comprises an IgM Cμ4 and IgM tailpiece domain.

7. The multimeric binding molecule of claim 6, wherein:
(a) the IgM heavy chain constant regions or fragments or variants thereof each further comprise a Cμ1 domain, a Cμ2 domain, a Cμ3 domain, or any combination thereof;
(b) the IgM heavy chain constant region is a human IgM constant region.
(c) each binding unit comprises two IgM heavy chains each comprising a VH situated amino terminal to the IgM constant region or fragment thereof, and two immunoglobulin light chains each comprising a VL situated amino terminal to an immunoglobulin light chain constant region.

8. The multimeric binding molecule of claim 6, wherein the IgM constant region comprises:
(a) SEQ ID NO: 35, SEQ ID NO: 36, or a multimerizing fragment thereof;
(b) a substitution relative to a wild-type human IgM constant region at position 310, 311, 313, and/or 315 of SEQ ID NO: 35 or SEQ ID NO: 36; or
(c) two or more substitutions relative to a wild-type human IgM constant region at positions 46, 209, 272, or 440 of SEQ ID NO: 35 or SEQ ID NO: 36.

9. The multimeric binding molecule of claim 6, which is pentameric, and further comprises a J-chain or functional fragment or variant thereof.

10. The multimeric binding molecule of claim 9, wherein the J-chain or functional fragment or variant thereof is a variant J-chain comprising one or more single amino acid substitutions, deletions, or insertions relative to a wild-type J-chain that can affect serum half-life of the multimeric binding molecule; and wherein the multimeric binding molecule comprising the variant J-chain exhibits an increased serum half-life upon administration to an animal relative to a reference multimeric binding molecule that is identical except for the one or more single amino acid substitutions, deletions, or insertions, and is administered in the same way to the same animal species.

11. The multimeric binding molecule of claim 9, wherein the J-chain or functional fragment thereof comprises:
(a) an amino acid substitution at the amino acid position corresponding to amino acid Y102 of the mature wild-type human J-chain (SEQ ID NO: 41);
(b) an amino acid substitution at the amino acid position corresponding to amino acid Y102 of the mature wild-type human J-chain (SEQ ID NO: 41), wherein the amino acid corresponding to Y102 of SEQ ID NO: 41 is substituted with alanine (A);
(c) an amino acid substitution at the amino acid position corresponding to amino acid Y102 of the mature wild-type human J-chain (SEQ ID NO: 41), wherein the J-chain is a variant human J-chain and comprises the amino acid sequence SEQ ID NO: 42; or
(d) an amino acid substitution at the amino acid position corresponding to amino acid N49, amino acid S51, or both N49 and S51 of the mature human J-chain (SEQ ID NO: 41), wherein a single amino acid substitution corresponding to position S51 of SEQ ID NO: 41 is not a threonine (T) substitution.

12. The multimeric binding molecule of claim 9, wherein the J-chain or functional fragment or variant thereof further comprises a heterologous polypeptide, wherein the heterologous polypeptide is fused to the J-chain or functional fragment or variant thereof via a peptide linker comprising at least 5 amino acids, but no more than 25 amino acids.

13. The multimeric binding molecule of claim 12, wherein the heterologous polypeptide is fused to the N-terminus of the J-chain or fragment or variant thereof, the C-terminus of the J-chain or fragment or variant thereof, or to both the N-terminus and C-terminus of the J-chain or fragment or variant thereof.

14. The multimeric binding molecule of claim 12, wherein the heterologous polypeptide comprises an antigen binding domain, and wherein:
(a) the antigen binding domain of the heterologous polypeptide is an antibody or antigen-binding fragment thereof;
(b) the antigen binding domain of the heterologous polypeptide is an antigen-binding fragment of an antibody, and the antigen-binding fragment comprises an Fab fragment, an Fab′ fragment, an F(ab′)2 fragment, an Fd fragment, an Fv fragment, a single-chain Fv (scFv) fragment, a disulfide-linked Fv (sdFv) fragment, or any combination thereof;
(c) the antigen binding domain of the heterologous polypeptide is an antigen-binding fragment of an antibody, and the antigen-binding fragment is a scFv fragment; and/or
(d) the antigen binding domain of the heterologous polypeptide is an antigen-binding fragment of an antibody, and the antigen binding domain binds ICOS Ligand (ICOSLG), ICOS (CD278), Interleukin 6 (IL6), CD28, CD3, CD80, CD86, Tumor Necrosis Factor Alpha (TNFa), or Fibroblast Activation Protein (FAP).

15. A composition comprising the multimeric binding molecule of any one of claims 1 to 14.

16. A polynucleotide comprising a nucleic acid sequence that encodes a polypeptide subunit of the binding molecule of any one of claims 1 to 14.

17. The polynucleotide of claim 16, wherein the polypeptide subunit comprises an IgM heavy chain constant region and at least an antibody VH portion of the PD-1-binding domain of the multimeric binding molecule; or comprises a light chain constant region and an antibody VL portion of the PD-1-binding domain of the multimeric binding molecule.

18. A composition comprising a polynucleotide comprising a nucleic acid sequence that encodes an IgM heavy chain constant region and at least an antibody VH portion of the PD-1-binding domain of the multimeric binding molecule of any one of claims 1 to 14, and a polynucleotide comprising a nucleic acid sequence that encodes a light chain constant region and an antibody VL portion of the PD-1-binding domain of the multimeric binding molecule of any one of claims 1 to 14, wherein the polynucleotides are on separate vectors or on a single vector.

19. The composition of claim 18, further comprising a polynucleotide comprising a nucleic acid sequence encoding a J chain, or a functional fragment thereof, or a functional variant thereof.

20. The vector or the vectors of claim 19.

21. A host cell comprising the composition of claim 18, wherein the host cell can express the binding molecule, or a subunit thereof.

22. A method of producing the binding molecule, comprising culturing the host cell of claim 21, and recovering the binding molecule.

23. A method for treating an autoimmune disorder, an inflammatory disorder, or a combination thereof in a subject in need of treatment comprising administering to the subject an effective amount of the multimeric binding molecule of any one of claims 1 to 14, wherein the multimeric binding molecule exhibits greater potency than an equivalent amount of a monomeric or dimeric binding molecule binding to the same binding partner.

24. A method for preventing transplantation rejection in a subject, comprising administering to the subject an effective amount of the multimeric binding molecule of any one of claims 1 to 14, wherein the multimeric binding molecule exhibits greater potency than an equivalent amount of a monomeric or dimeric binding molecule binding to the same binding partner, and wherein the subject is a transplantation recipient.

25. The method of claim 23, wherein the subject is human.


SEQUENCE LISTING (50 sequences)

SEQ ID NO: 1 - protein, 119 aa, Artificial Sequence
  Description of Artificial Sequence Synthetic polypeptide
QVQLVQSGAEVKKPGASVKVSCKVSGYSLSKYDMSWVRQAPGKGLEWMGIIYTSGYTDYAQKFQGRVTMTEDTSTDTAYMELSSLRSEDTAVYYCATGNPYYTNGFNSWGQGTLVTVSS

SEQ ID NO: 2 - protein, 110 aa, Artificial Sequence
  Description of Artificial Sequence Synthetic polypeptide
DIQMTQSPSSLSASVGDRVTITCQASQSPNNLLAWYQQKPGKAPKLLIYGASDLPSGVPSRFSGSGSGTDFTLTISSLQPEDFATYYCQNNYYVGPVSYAFGGGTKVEIK

SEQ ID NO: 3 - protein, 121 aa, Artificial Sequence
  Description of Artificial Sequence Synthetic polypeptide
QVQLVQSGSELKKPGASVKVSCKASGYTFTDYSMHWVRQAPGQGLEWMGWINIETGEPTYAQGFTGRFVFSLDTSVSTAYLQISSLKAEDTAVYFCARDYYGTYFYAMDYWGQGTLVTVSS

SEQ ID NO: 4 - protein, 108 aa, Artificial Sequence
  Description of Artificial Sequence Synthetic polypeptide
EIVLTQSPATLSLSPGERATLSCTASSSVSSSYLHWYQQKPGLAPRLLIYSTSNLASGIPDRFSGSGSGTDYTLTISRLEPEDFAVYYCHQYHRSPLTFGQGTKLEIK

SEQ ID NO: 5 - protein, 122 aa, Artificial Sequence
  Description of Artificial Sequence Synthetic polypeptide
QVTLKESGPGLLQPSQTLSLTCSFSGFSLSTSGMGVSWIRQPSGKGLEWLAHIYWDDDKRYNPSLKSRLTISKDTSSNQVFLKITSVDTADTGTYYCVRKGYYDYGYVMDYWGQGTTVTVSS

SEQ ID NO: 6 - protein, 112 aa, Artificial Sequence
  Description of Artificial Sequence Synthetic polypeptide
DIVMTQAALSNPVTLGTSASISCRSSKSLLHSNGITYLNWYLQKPGQSPQLLIYQMSNLASGVPDRFSSSGSGTDFTLRISRVEAEDVGVYYCAQNLELPLTFGSGTKLEMK

SEQ ID NO: 7 - protein, 120 aa, Artificial Sequence
  Description of Artificial Sequence Synthetic polypeptide
QVQLQQSGAELVKPGASVKLSCKASGYTFTSYDINWVRQRPEQGLEWIGWIFPGDGSTKYNEKFKGKATLTTDKSSSTAYMQFSRLTSEDSAVYFCARGGMRQLGRFVYWGQGTTLTVSS

SEQ ID NO: 8 - protein, 107 aa, Artificial Sequence
  Description of Artificial Sequence Synthetic polypeptide
DIVLTQSPSSLSASLGERVSLTCRASQEISGYLSWLQQKPDGTIKRLIYAASTLDSGVPKRFSGSRSGSDYSLTISSLESEDFADYYCLQYASNPYTFGGGTKLEIK

SEQ ID NO: 9 - protein, 119 aa, Artificial Sequence
  Description of Artificial Sequence Synthetic polypeptide
EVQLQQSGAELVKPGASVKLSCTASGFNVKDTYFHWVKQRPDQGLEWIGRIVSANGDTKYAPKLQDKATITTDTSSNTAYLQLSRLTSEDTAVYYCVLIYYGFEEGDFWGQGTTLTVSS

SEQ ID NO: 10 - protein, 107 aa, Artificial Sequence
  Description of Artificial Sequence Synthetic polypeptide
DIVMTQSPSSLSASLGDTITITCHASQNINVWLSWYQQKPGNVPKLLIYKASNLHTGVPSRFSGSGSGTGFTLNISSLQPEDIATYYCQQGQSFPLTFGAGTKLELK

SEQ ID NO: 11 - protein, 117 aa, Artificial Sequence
  Description of Artificial Sequence Synthetic polypeptide
QVQLVQSGAEVKKPGASVKVSCKASGYTFTTYWMHWVRQAPGQGLEWMGEINPNEGGINYAQKFQGRVTLTVDKSISTAYMELSRLRSDDTAVYYCTIDYYDYGGYWGQGTLVTVSS

SEQ ID NO: 12 - protein, 107 aa, Artificial Sequence
  Description of Artificial Sequence Synthetic polypeptide
DIQMTQSPSSLSASVGDRVTITCKASQNVGTNVAWYQQKPEKAPKSLIYSASYRYSGVPSRFSGSGSGTDFTLTISSLQPEDFATYYCQQYNIYPYTFGQGTKLEIK

SEQ ID NO: 13 - protein, 119 aa, Artificial Sequence
  Description of Artificial Sequence Synthetic polypeptide
EVQLQESGPGLVKPSQTLSLTCTVTGYSITSDYAWNWIRQPPGKKLEWMGYINYSGSTSYNPSLKSRVTISRDTSKNQFSLKLSSVTAADTAVYYCARWIGSSAWYFDVWGQGTLVTVS

SEQ ID NO: 14 - protein, 112 aa, Artificial Sequence
  Description of Artificial Sequence Synthetic polypeptide
DVLMTQTPLSLSVTPGQPASISCRSGQNIVHSNGNTYLEWYLQKPGQSPKLLIYKVSNRFFGVPDRISGSGSGTDFTLKISRVEAEDVGVYFCFQGSHVPFTFGQGTKLEIK

SEQ ID NO: 15 - protein, 119 aa, Artificial Sequence
  Description of Artificial Sequence Synthetic polypeptide
EVQLVQSGAEVKKPGASVKVSCKAFGYTFTTYPIEWMRQAHGQGLEWIGNFHPYNDDTKYNEKFKGRVTMTVDKSTTTVYMELSSLRSEDTAVYYCARENYGSHGGFVYWGQGTLVTVS

SEQ ID NO: 16 - protein, 108 aa, Artificial Sequence
  Description of Artificial Sequence Synthetic polypeptide
ENVLTQSPFSLSASVGDRVTITCRASSSVISSYLHWYQQKPAKAPKLWIYSTSNLASGVPDRFSGSGSGTSYTLTISSLQPEDFATYYCQQYNSYPLTFGGGTKVEIK

SEQ ID NO: 17 - protein, 119 aa, Artificial Sequence
  Description of Artificial Sequence Synthetic polypeptide
EVQLVQSGAEVKKPGASVKVSCKAFGYTFTTYPIEWMRQAHGQGLEWIGNFHPYNDDTKYNEKFKGRVTMTRDTSTSTVYMELSSLRSEDTAVYYCARENYGSHGGFVYWGQGTLVTVS

SEQ ID NO: 18 - protein, 108 aa, Artificial Sequence
  Description of Artificial Sequence Synthetic polypeptide
ENVMTQSPFSLSASVGDRVTITCRASSSVISSYLHWYQQKPAKAPKLFIYSTSNLASGVPSRFSGSGSGTDYTLTISSLQPEDFATYYCQQYNSYPLTFGGGTKVEIK

SEQ ID NO: 19 - protein, 119 aa, Artificial Sequence
  Description of Artificial Sequence Synthetic polypeptide
EVQLVQSGAEVKKPGASVKVSCKAFGYTFTTYPIEWVRQAPGQGLEWMGNFHPYNDDTKYNEKFKGRVTMTRDTSTSTVYMELSSLRSEDTAVYYCARENYGSHGGFVYWGQGTLVTVS

SEQ ID NO: 20 - protein, 108 aa, Artificial Sequence
  Description of Artificial Sequence Synthetic polypeptide
ENVLTQSPGTLSLSPGERATLSCRASSSVISSYLHWYQQKPGQAPRLWIYSTSNLASGVPDRFSGSGSGTSYTLTISRLEPEDFATYYCQQYNSYPLTFGGGTKVEIK

SEQ ID NO: 21 - protein, 119 aa, Artificial Sequence
  Description of Artificial Sequence Synthetic polypeptide
EVQLVQSGAEVKKPGSSVKVSCKAFGYTFTTYPIEWMRQAHGQGLEWIGNFHPYNDDTKYNEKFKGRVTITVDKSTTTVYMELSSLRSEDTAVYYCARENYGSHGGFVYWGQGTLVTVS

SEQ ID NO: 22 - protein, 108 aa, Artificial Sequence
  Description of Artificial Sequence Synthetic polypeptide
ENVLTQSPFSLSASVGDRVTITCRASSSVISSYLHWYQQKPAKAPKLFIYSTSNLASGVPSRFSGSGSGTDYTLTISSLQPEDFATYYCQQYNSYPLTFGGGTKVEIK

SEQ ID NO: 23 - protein, 119 aa, Artificial Sequence
  Description of Artificial Sequence Synthetic polypeptide
EVQLVQSGAEVKKPGSSVKVSCKAFGYTFTTYPIEWMRQAHGQGLEWIGNFHPYNDDTKYNEKFKGRVTITADKSTSTAYMELSSLRSEDTAVYYCARENYGSHGGFVYWGQGTLVTVS

SEQ ID NO: 24 - protein, 119 aa, Artificial Sequence
  Description of Artificial Sequence Synthetic polypeptide
EVQLVQSGAEVKKPGSSVKVSCKASGYTFTTYPIEWVRQAPGQGLEWMGNFHPYNDDTKYNEKFKGRVTITADKSTSTAYMELSSLRSEDTAVYYCARENYGSHGGFVYWGQGTLVTVS

SEQ ID NO: 25 - protein, 128 aa, Artificial Sequence
  Description of Artificial Sequence Synthetic polypeptide
QVQLQESGPGVVKPSGTLSLTCAISGGSIGSGGSIRSTRWWSWVRQSPGKGLEWIGEIYHSGSTNYNPSLKSRVTISLDKSRNHFSLRLNSVTAADTAVYYCARQDYGDSGDWYFDLWGKGTMVTVSS

SEQ ID NO: 26 - protein, 110 aa, Artificial Sequence
  Description of Artificial Sequence Synthetic polypeptide
NFMLTQPHSVSESPGKTVTISCTRSSGSIASNSVQWYQQRPGSSPTTVIYEDNQRPSGVPDRFSGSIDSSSNSASLTVSGLKTEDEADYYCQSSDSSAVVFGSGTKLTVL

SEQ ID NO: 27 - protein, 119 aa, Artificial Sequence
  Description of Artificial Sequence Synthetic polypeptide
EVQLVQSGAEVKKPGASVKVSCKASGYRFTSYGISWVRQAPGQGLEWMGWISAYNGNTNYAQKLQGRVTMTTDTSTNTAYMELRSLRSDDTAVYYCARDADYSSGSGYWGQGTLVTVSS

SEQ ID NO: 28 - protein, 108 aa, Artificial Sequence
  Description of Artificial Sequence Synthetic polypeptide
SYELTQPPSVSVSPGQTARITCSGDALPKQYAYWYQQKPGQAPVMVIYKDTERPSGIPERFSGSSSGTKVTLTISGVQAEDEADYYCQSADNSITYRVFGGGTKVTVL

SEQ ID NO: 29 - protein, 119 aa, Artificial Sequence
  Description of Artificial Sequence Synthetic polypeptide
QVQLVQSGAEVKKPGASVRVSCKASGYTLTSYYIHWVRQAPGQGLEWMGIINPRGATISYAQKFQGRVTMTRDTSTSTVYMELRNLKSEDTALYYCATAGIYGFDFDYWGRGTLVTVSS

SEQ ID NO: 30 - protein, 111 aa, Artificial Sequence
  Description of Artificial Sequence Synthetic polypeptide
QSALTQPASVSGSPGQSITISCTGTSNDVGGYNYVSWYQHHPGKAPKLIIYDVTNRPSGVSDRFSGSKSGNTASLTISGLLAEDEGDYYCSSYTIVTNFEVLFGGGTKLTV

SEQ ID NO: 31 - protein, 127 aa, Artificial Sequence
  Description of Artificial Sequence Synthetic polypeptide
QVQLQESGPGLVKPSQTLSLTCTVSGGSISSGAYYWSWIRQHPGKGLEWIGYIYYNGNTYYNPSLRSLVTISVDASKNQFSLKLSSVTAADTAVYYCARASDYVWGGYRYMDAFDIWGRGTLITVSS

SEQ ID NO: 32 - protein, 112 aa, Artificial Sequence
  Description of Artificial Sequence Synthetic polypeptide
QSVLTQPPSASGTPGQRVTISCSGSNSNIGSNSVNWYQQLPGTAPKLLIYGNNQRPSGVPDRFSGSKSGTSASLAISGLQSENEADYYCAAWDDSLNGPVFGRGTKVTVLGE

SEQ ID NO: 33 - protein, 125 aa, Artificial Sequence
  Description of Artificial Sequence Synthetic polypeptide
GAHSEVQLVQSGGGVVQPGRSLRLSCAASGFTFSSYWCDRMSWVRQAPGKGLEWVSAISGSGGSTYYADSVKGRFTISRDNSKNTLYLQMNSLRAEDTAVYYCAKENWGSYFDLWGQGTTVTVSS

SEQ ID NO: 34 - protein, 112 aa, Artificial Sequence
  Description of Artificial Sequence Synthetic polypeptide
GVHSDIVMTQSPSTLSASVGDRVTITCRASQGISSWLAWYQQKPGRAPKVLIYKASTLESGVPSRFSGSGSGTDFTLTISSLQPEDFATYYCQQSYSTPWTFGQGTKLEIKR

SEQ ID NO: 35 - protein, 453 aa, Homo sapiens [sequence omitted]

SEQ ID NO: 36 - protein, 453 aa, Homo sapiens
GSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 37 - protein, 353 aa, Homo sapiens
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCPVPSTPPTPSPSTPPTPSPSCCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 38 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCPVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRMAGKPTHVNVSVVMAEVDGTCY

SEQ ID NO: 39 - protein, 764 aa, Homo sapiens
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGGSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRALVSTLVPLGLVLAVGAVAVGVARARHRKNVDRVSIRSYRTDISMSDFENSREFGANDNMGASSITQETSLGGKEEFVATTESTTETKEPKKAKRSSKEEAEMAYKDFLLQSSTVAAEAQDGPQEA

SEQ ID NO: 40 - protein, 159 aa, Homo sapiens
MKNHLLFWGVLAVFIKAVHVKAQEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 41 - protein, 137 aa, Homo sapiens
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 42 - protein, 137 aa, Artificial Sequence
  Description of Artificial Sequence Synthetic polypeptide
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCATYDRNKCYTAVVPLVYGGETKMVETALTPDACYPD

SEQ ID NO: 43 - protein, 5 aa, Artificial Sequence
  Description of Artificial Sequence Synthetic peptide
GGGGS

SEQ ID NO: 44 - protein, 10 aa, Artificial Sequence
  Description of Artificial Sequence Synthetic peptide
GGGGSGGGGS

SEQ ID NO: 45 - protein, 15 aa, Artificial Sequence
  Description of Artificial Sequence Synthetic peptide
GGGGSGGGGSGGGGS

SEQ ID NO: 46 - protein, 20 aa, Artificial Sequence
  Description of Artificial Sequence Synthetic peptide
GGGGSGGGGSGGGGSGGGGS

SEQ ID NO: 47 - protein, 25 aa, Artificial Sequence
  Description of Artificial Sequence Synthetic peptide
GGGGSGGGGSGGGGSGGGGSGGGGS

SEQ ID NO: 48 - protein, 340 aa, Homo sapiens
ASPTSPKVFPLSLDSTPQDGNVVVACLVQGFFPQEPLSVTWSESGQNVTARNFPPSQDASGDLYTTSSQLTLPATQCPDGKSVTCHVKHYTNSSQDVTVPCRVPPPPPCCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKSGNTFRPEVHLLPPPSEELALNELVTLTCLARGFSPKDVLVRWLQGSQELPREKYLTWASRQEPSQGTTTYAVTSILRVAAEDWKKGETFSCMVGHEALPLAFTQKTIDRMAGKPTHINVSVVMAEADGTCY

SEQ ID NO: 49 - protein, 113 aa, Artificial Sequence [sequence omitted]

SEQ ID NO: 50 - protein, 107 aa, Artificial Sequence [sequence omitted]